(19) United States
(12) Patent Application Publication
(10) Pub. No.: US 2016/0213300 A1
(43) Pub. Date: Jul. 28, 2016

(54) One or More Machines/Articles/Compositions/Processes Related to Traumatic Brain Injuries

(71) Applicant: Elwha LLC, Bellevue, WA (US)

(72) Inventors: Paul G. Allen, Mercer Island, WA (US); Philip V. Bayly, St. Louis, MO (US); David Lozoff Brody, St. Louis, MO (US); Jesse R. Cheatham, III, Seattle, WA (US); Richard Glen Ellenbogen, Seattle, WA (US); Roderick A. Hyde, Redmond, WA (US); Muriel Y. Ishikawa, Livermore, CA (US); Eric C. Leuthardt, St. Louis, MO (US); Nathan P. Myhrvold, Medina, WA (US); Robert C. Petroski, Seattle, WA (US); Raul Radovitzky, Bedford, MA (US); Elizabeth A. Sweeney, Seattle, WA (US); Clarence T. Tegreene, Mercer Island, WA (US); Nicholas W. Touran, Seattle, WA (US); Lowell L. Wood, JR., Bellevue, WA (US); Victoria Y. H. Wood, Livermore, CA (US); Richard T. Lord, Gig Harbor, WA (US); Robert W. Lord, Seattle, WA (US)

(21) Appl. No.: 15/006,577

(22) Filed: Jan. 26, 2016

Related U.S. Application Data
(63) Continuation-in-part of application No. 14/929,338, filed on Oct. 31, 2015.
(60) Provisional application No. 62/108,047, filed on Jan. 26, 2015.

(51) Int. Cl. A61B 5/00; A61B 5/11

(52) U.S. Cl. CPC A61B 5/4064; A61B 5/11; A61B 5/0004; A61B 5/6803; A61B 2562/0219; A61B 2503/10

(57) ABSTRACT

Computationally implemented methods and systems include electronically collecting player acceleration data associated with a head of a sports player, electronically monitoring game time player status data of the sports player, and electronically obtaining player brain injury status data associated with brain injury status of the sports player based at least in part upon the player acceleration data and the game time player status data of the sports player. In addition to the foregoing, other aspects are described in the claims, drawings, and text.

### CROSS-REFERENCE TO RELATED APPLICATIONS

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

For purposes of the USPTO extra-statutory requirements, the present application constitutes a non-provisional of U.S. Patent Application No. 62/108,047, entitled ONE OR MORE MACHINES/ARTICLES/COMPOSITIONS/PROCESSES RELATED TO TRAUMATIC BRAIN INJURIES, naming Paul G. Allen, Philip V. Bayly, David L. Brody, Jesse R. Cheatham, III, Richard G. Ellenbogen, Roderick A. Hyde, Muriel Y. Ishikawa, Eric C. Leuthardt, Richard T. Lord, Robert W. Lord, Nathan P. Myhrvold, Robert C. Petroski, Raul Radovitzky, Elizabeth A. Sweeney, Clarence T. Tegreene, Nicholas W. Touran, Lowell L. Wood, Jr., and Victoria Y. H. Wood, filed 26 Jan. 2015 with attorney docket no. 0414-002-014-PR0001, which is currently co-pending or is an application of which a currently co-pending application is entitled to the benefit of the filing date.

For purposes of the USPTO extra-statutory requirements, the present application constitutes a continuation-in-part of U.S. patent application Ser. No. 14/929,338, entitled ONE OR MORE MACHINES/ARTICLES/COMPOSITIONS/PROCESSES RELATED TO TRAUMATIC BRAIN INJURIES, naming Paul G. Allen, Philip V. Bayly, David L. Brody, Jesse R. Cheatham, III, Richard G. Ellenbogen, Roderick A. Hyde, Muriel Y. Ishikawa, Eric C. Leuthardt, Richard T. Lord, Robert W. Lord, Nathan P. Myhrvold, Robert C. Petroski, Raul Radovitzky, Elizabeth A. Sweeney, Clarence T. Tegreene, Nicholas W. Touran, Lowell L. Wood, Jr., and Victoria Y. H. Wood, filed 31 Oct. 2015 with attorney docket no. 0414-002-001-000000, which is currently co-pending or is an application of which a currently co-pending application is entitled to the benefit of the filing date.

### RELATED APPLICATIONS

None.

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The USPTO further has provided forms for the Application Data Sheet which allow automatic loading of bibliographic data but which require identification of each application as a continuation, continuation-in-part, or divisional of a parent application. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above and in any ADS filed in this application, but expressly points out that such designation(s) are not to be construed in any way as any type of commentary and/or admission as to whether or not the present application contains any new matter in addition to the matter of its parent application(s).

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### BACKGROUND

This application is related to one or more machines, articles, compositions, and processes related to traumatic brain injuries such as regarding sensing, testing, status, location, and access of players of sports including during their games.

### SUMMARY

In one or more various aspects, a method includes, but is not limited to electronically collecting player acceleration data associated with a head of a sports player; electronically monitoring game time player status data of the sports player; and electronically obtaining player brain injury status data associated with brain injury status of the sports player based at least in part upon the player acceleration data and the game time player status data of the sports player. In addition to the foregoing, other method aspects are described in the claims, drawings, and text forming a part of the disclosure set forth herein.

In one or more various aspects, one or more related systems may be implemented in machines, compositions of matter, or manufactures of systems, limited to patentable subject matter under 35 U.S.C. 101. The one or more related systems may include, but are not limited to, circuitry and/or programming for carrying out the herein-referenced method aspects. The circuitry and/or programming may be virtually any combination of hardware, software, and/or firmware configured to effect the herein—referenced method aspects depending upon the design choices of the system designer, and limited to patentable subject matter under 35 USC 101.

In one aspect, a system includes, but is not limited to means for electronically collecting player acceleration data associated with a head of a sports player; means for electronically monitoring game time player status data of the sports player; and means for electronically obtaining player brain injury status data associated with brain injury status of the sports player based at least in part upon the player acceleration data and the game time player status data of the sports player. In addition to the foregoing, other system aspects are described in the claims, drawings, and text forming a part of the disclosure set forth herein.

In one aspect, a system includes, but is not limited to circuitry for electronically collecting player acceleration data associated with a head of a sports player; circuitry for electronically monitoring game time player status data of the sports player; and circuitry for electronically obtaining player brain injury status data associated with brain injury status of the sports player based at least in part upon the player acceleration data and the game time player status data of the sports player. In addition to the foregoing, other system aspects are described in the claims, drawings, and text forming a part of the disclosure set forth herein.

In one aspect, a system includes, but is not limited to Error! Reference source not found. module configured to operate in accordance with electronically collecting player acceleration data associated with a head of a sports player; Error! Reference source not found. module configured to operate in accordance with electronically monitoring game time player status data of the sports player; Error! Reference source not found. module configured to operate in accordance with electronically obtaining player brain injury status data associated with brain injury status of the sports player based at least in part upon the player acceleration data and the game time player status data of the sports player. In addition to the foregoing, other system aspects are described in the claims, drawings, and text forming a part of the disclosure set forth herein.

In one aspect, a computer program product may be expressed as an article of manufacture that bears instructions including, but not limited to one or more instructions for electronically collecting player acceleration data associated with a head of a sports player; one or more instructions for electronically monitoring game time player status data of the sports player; and one or more instructions for electronically obtaining player brain injury status data associated with brain injury status of the sports player based at least in part upon the player acceleration data and the game time player status data of the sports player. In addition to the foregoing, other computer program product aspects are described in the claims, drawings, and text forming a part of the disclosure set forth herein.

In one aspect, a system includes, but is not limited to one or more computing devices; and one or more instructions when executed on the one or more computing devices cause the one or more computing devices to perform electronically collecting player acceleration data associated with a head of a sports player; electronically monitoring game time player status data of the sports player; and electronically obtaining player brain injury status data associated with brain injury status of the sports player based at least in part upon the player acceleration data and the game time player status data of the sports player. In addition to the foregoing, other computer program product aspects are described in the claims, drawings, and text forming a part of the disclosure set forth herein.

In addition to the foregoing, various other method and/or system and/or program product aspects are set forth and described in the teachings such as text (e.g., claims and/or detailed description) and/or drawings of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent by reference to the detailed description, the corresponding drawings, and/or in the teachings set forth herein.

### BRIEF DESCRIPTION OF THE FIGURES

For a more complete understanding of embodiments, reference now is made to the following descriptions taken in connection with the accompanying drawings. The use of the same symbols in different drawings typically indicates similar or identical items, unless context dictates otherwise. The illustrative embodiments described in the detailed description, drawings, and claims are not meant to be limiting. Other embodiments may be utilized, and other changes may be made, without departing from the spirit or scope of the subject matter presented here.

FIG. 1 shows a high-level system diagram of one or more exemplary environments in which transactions and potential transactions may be carried out, according to one or more embodiments. FIG. 1 forms a partially schematic diagram of an environment(s) and/or an implementation(s) of technologies described herein when FIGS. 1-A through 1-F are stitched together in the manner shown in FIG. 1, which is reproduced below in table format.

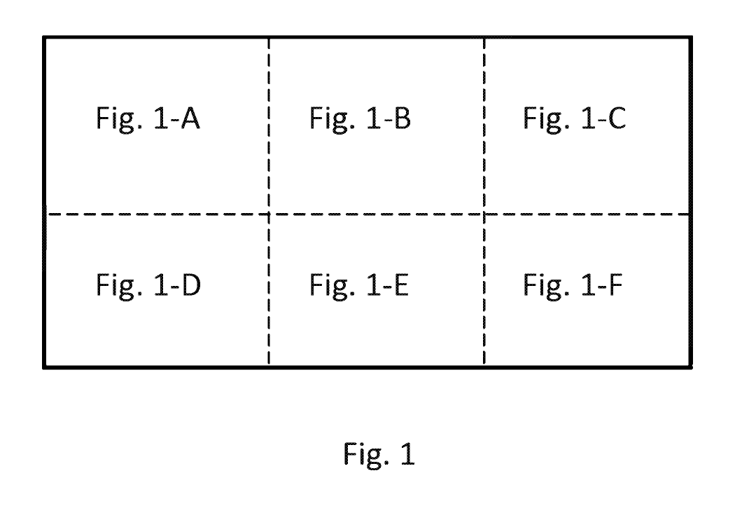

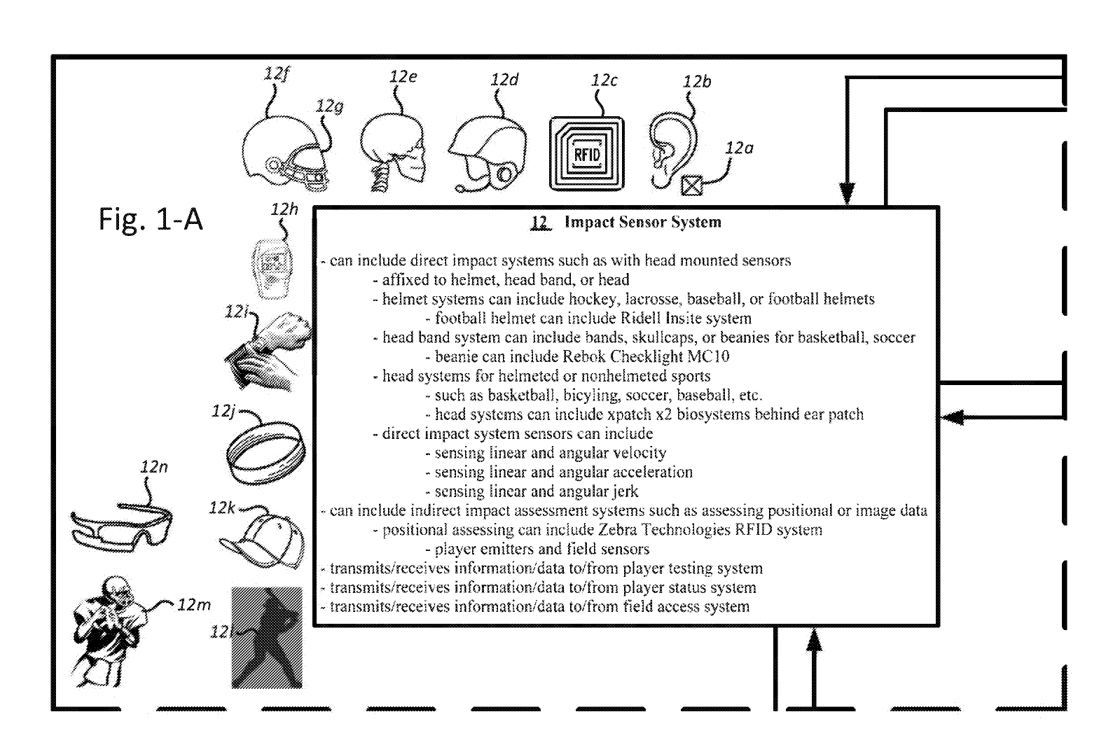

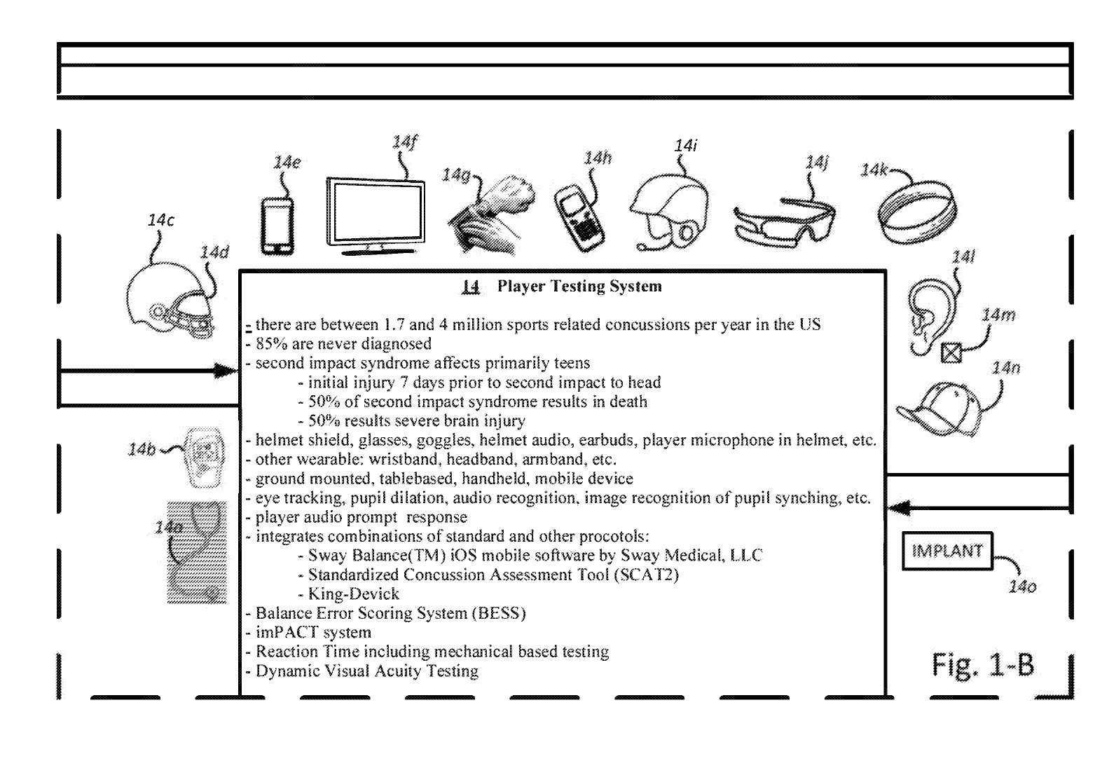

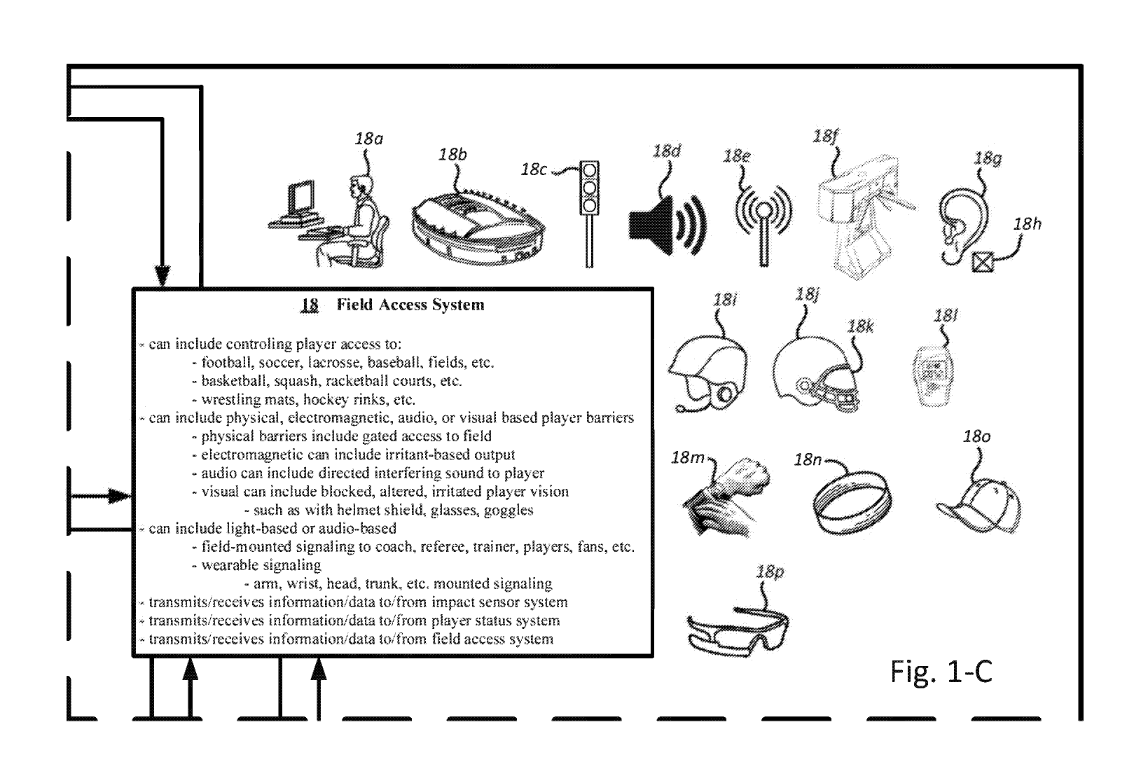

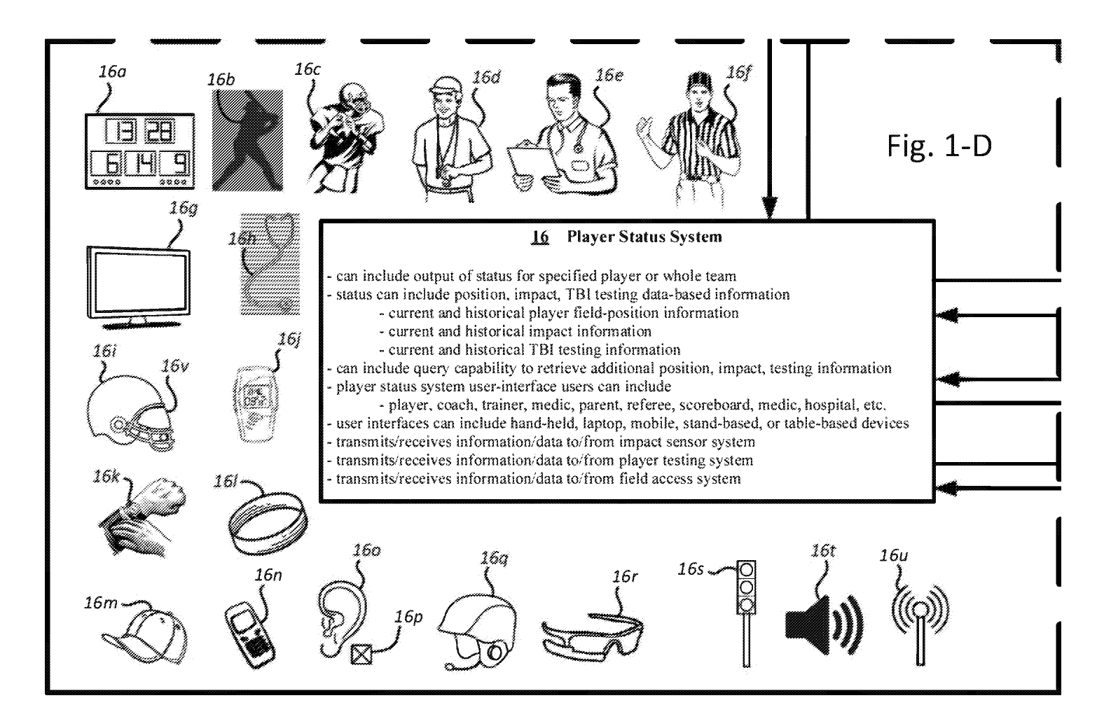

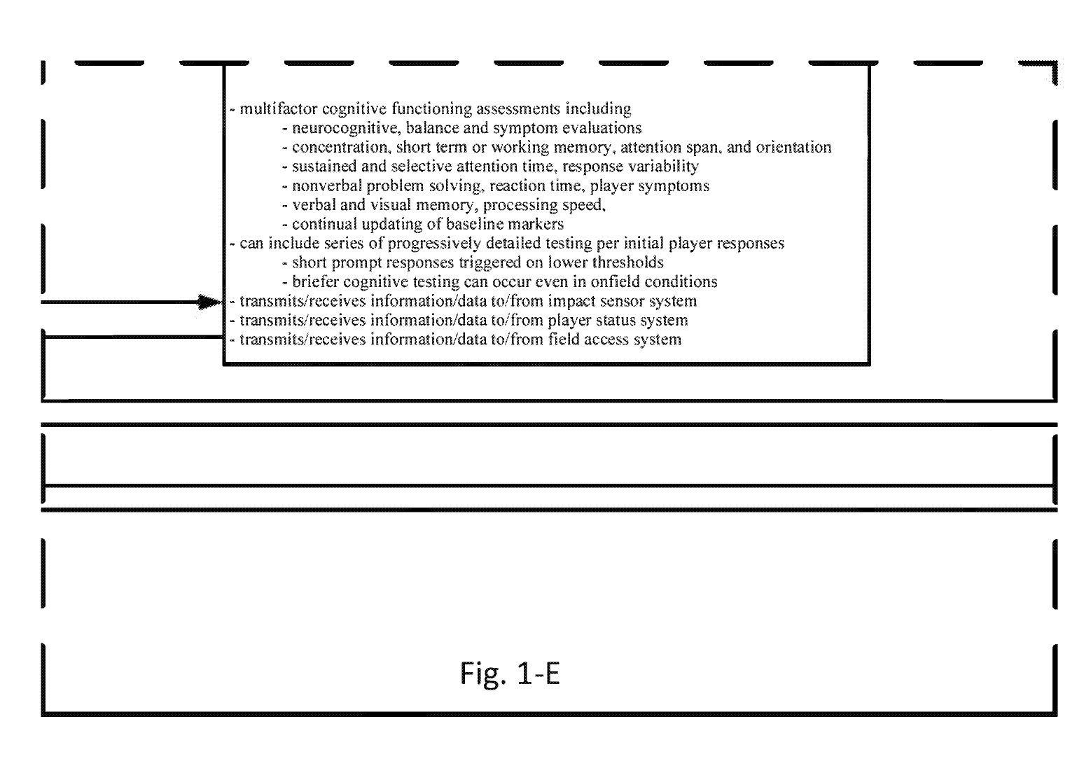

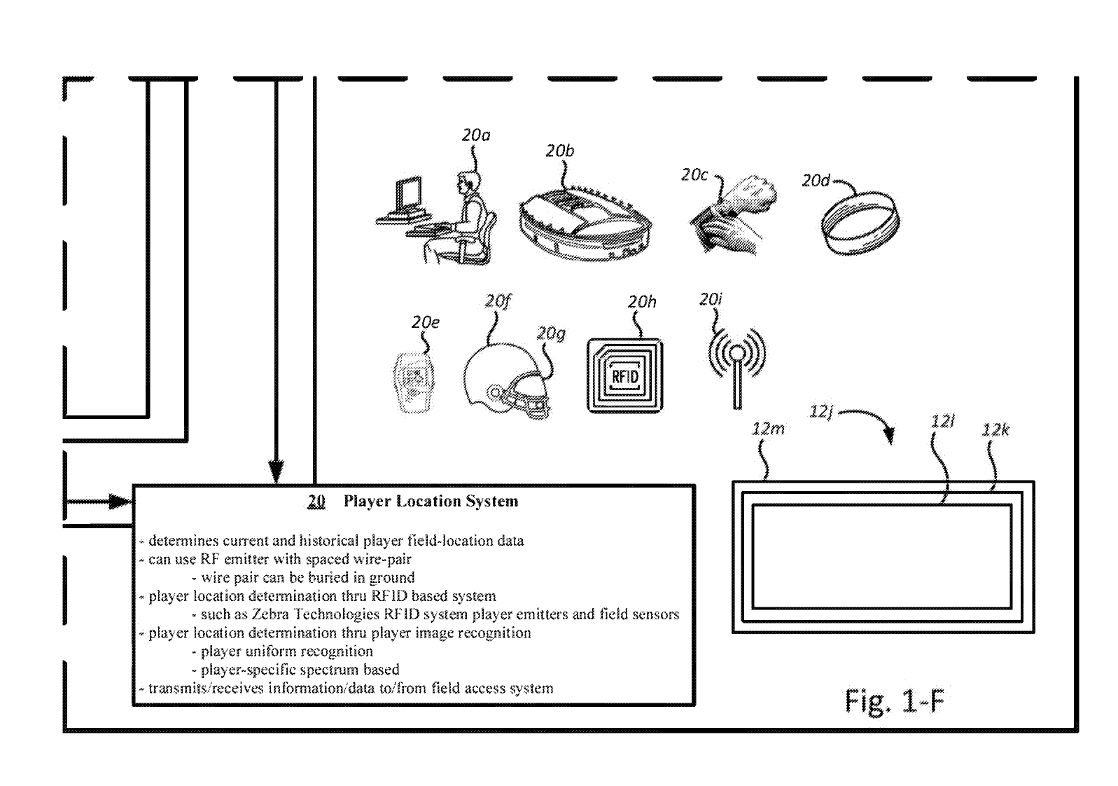

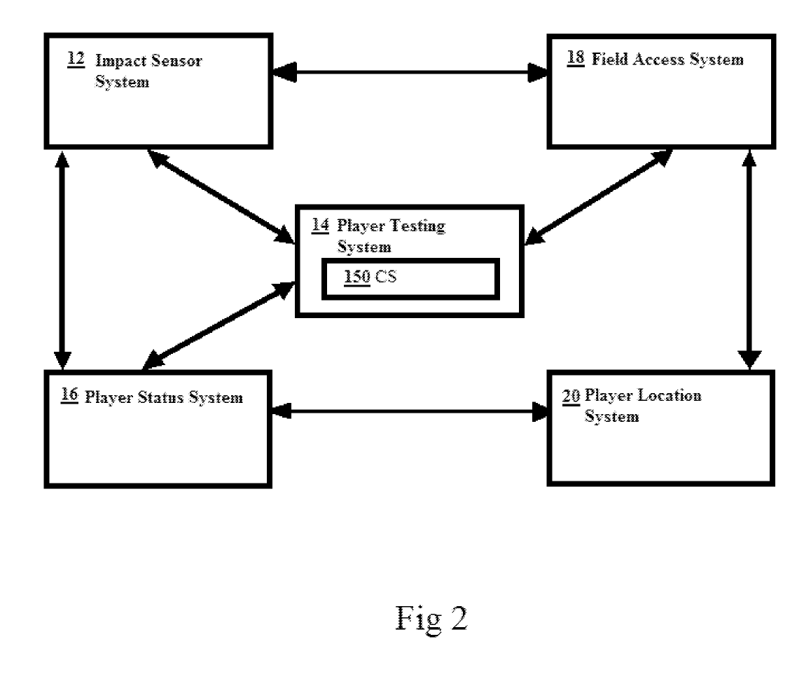

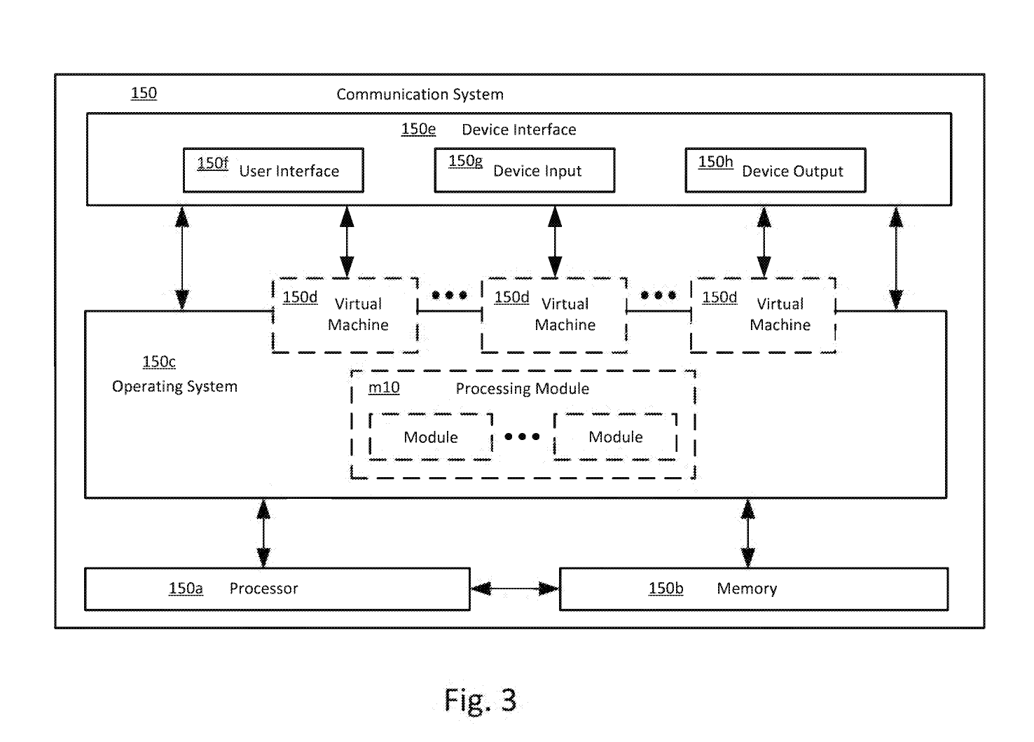

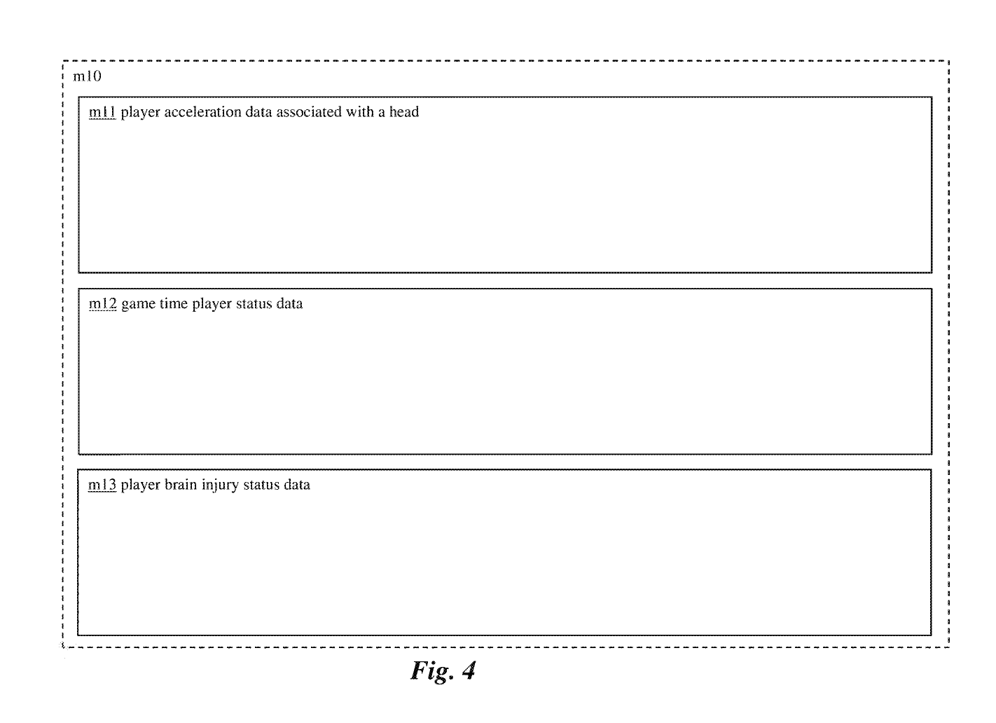

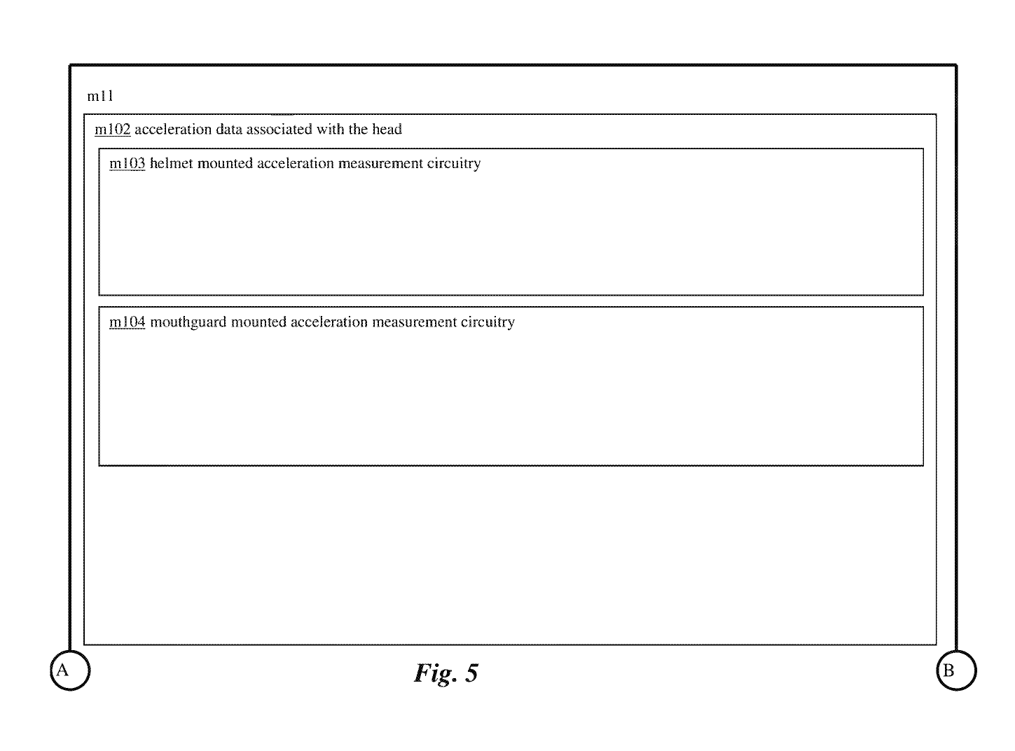

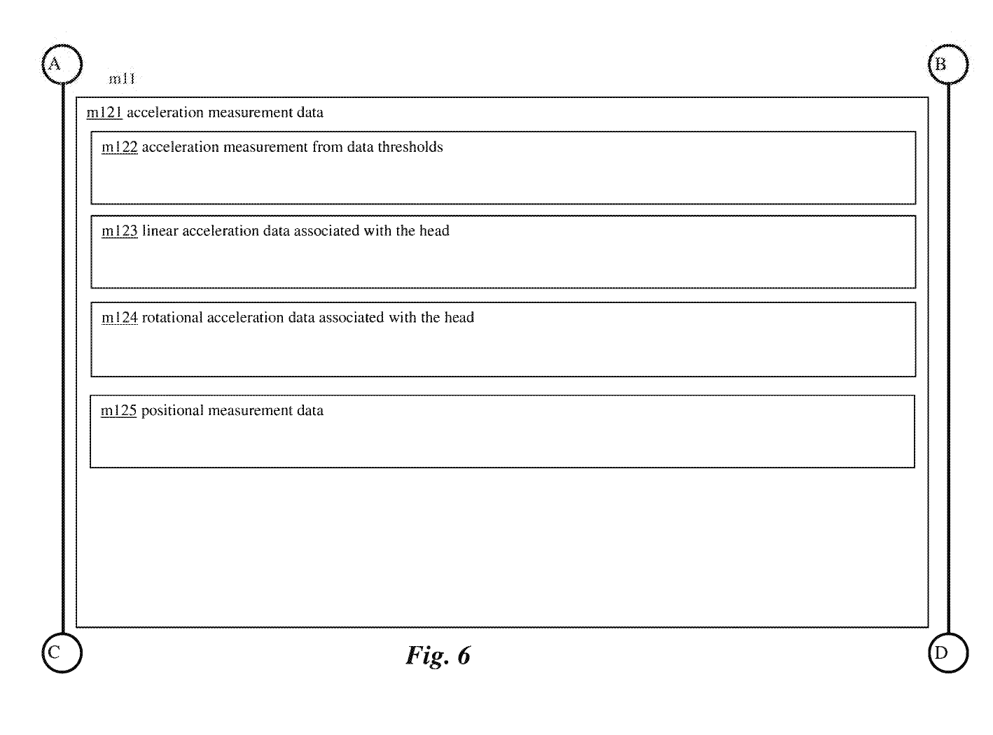

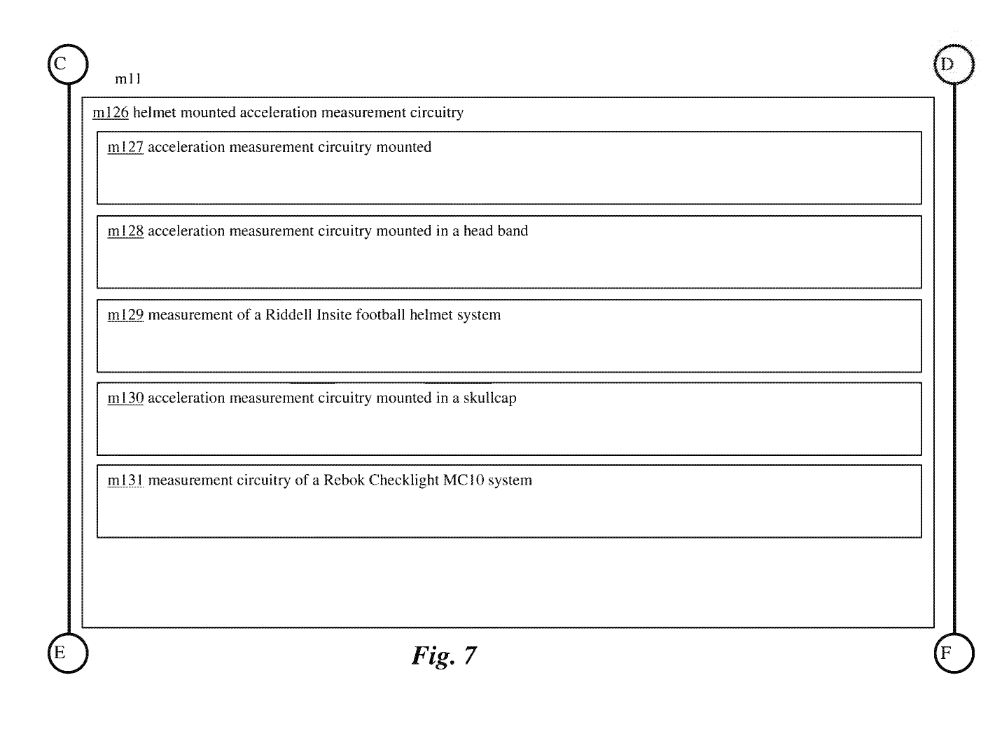

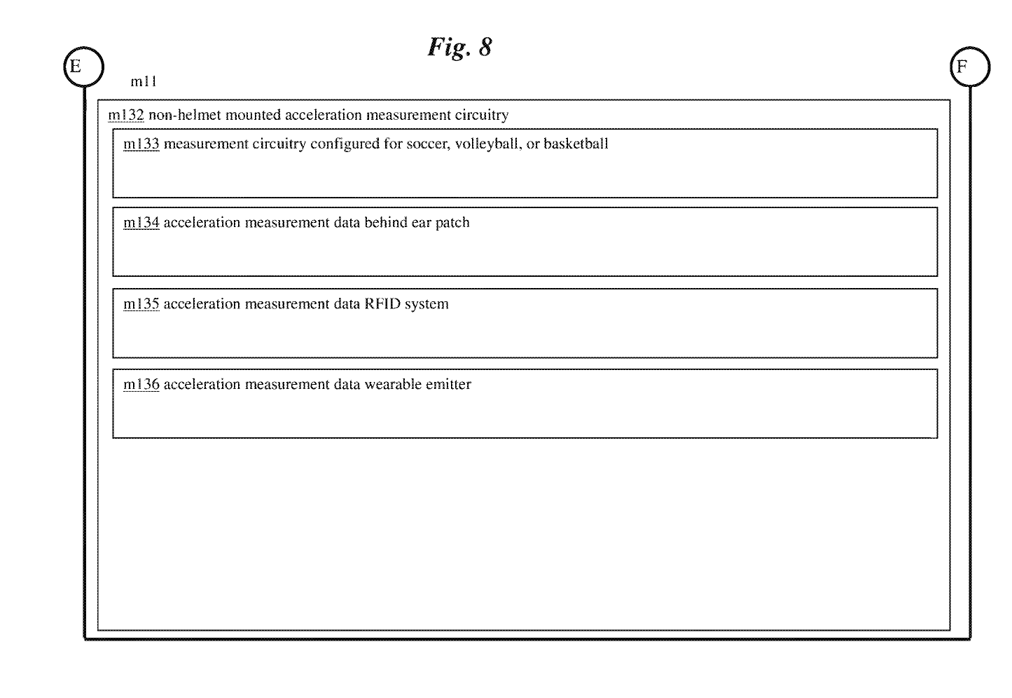

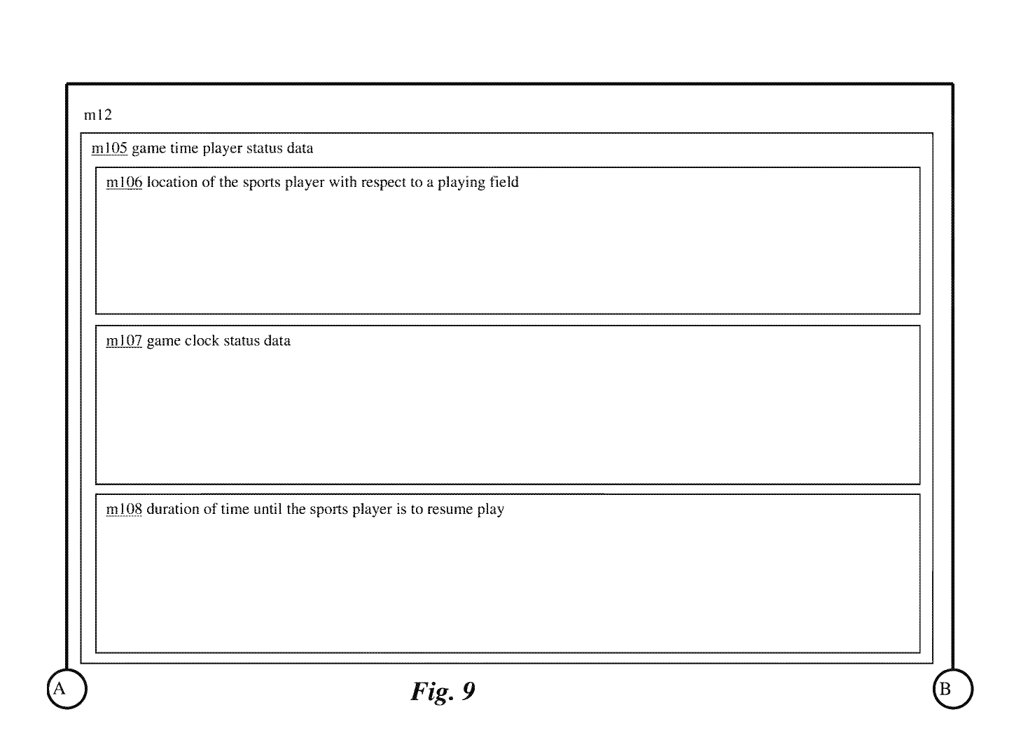

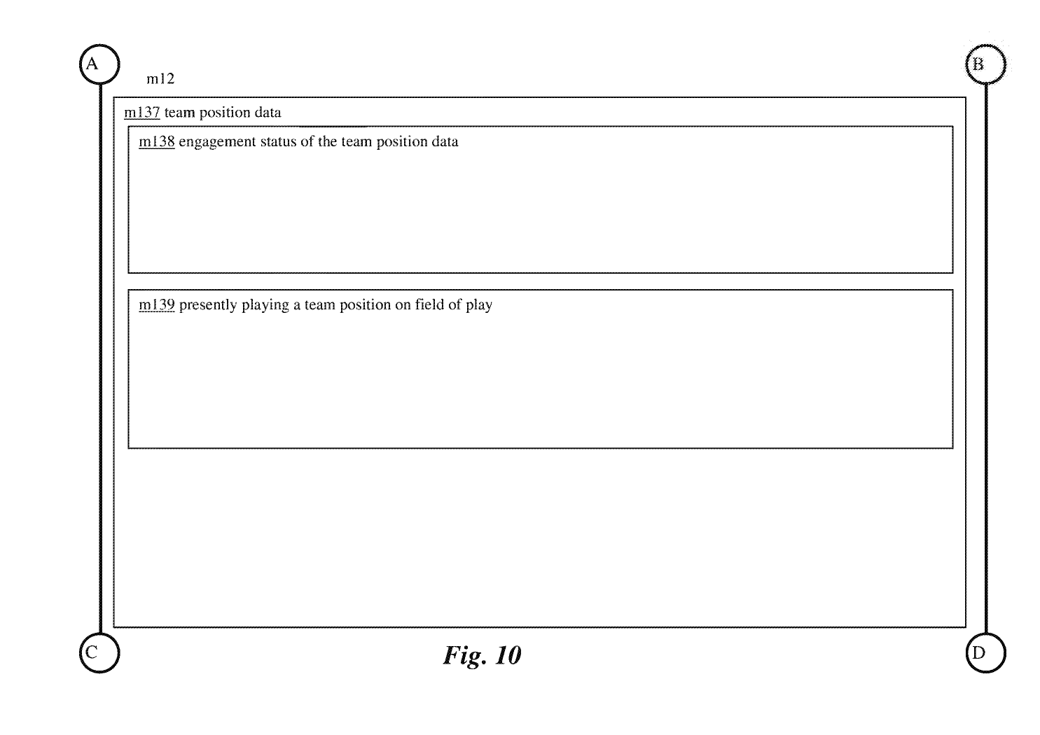

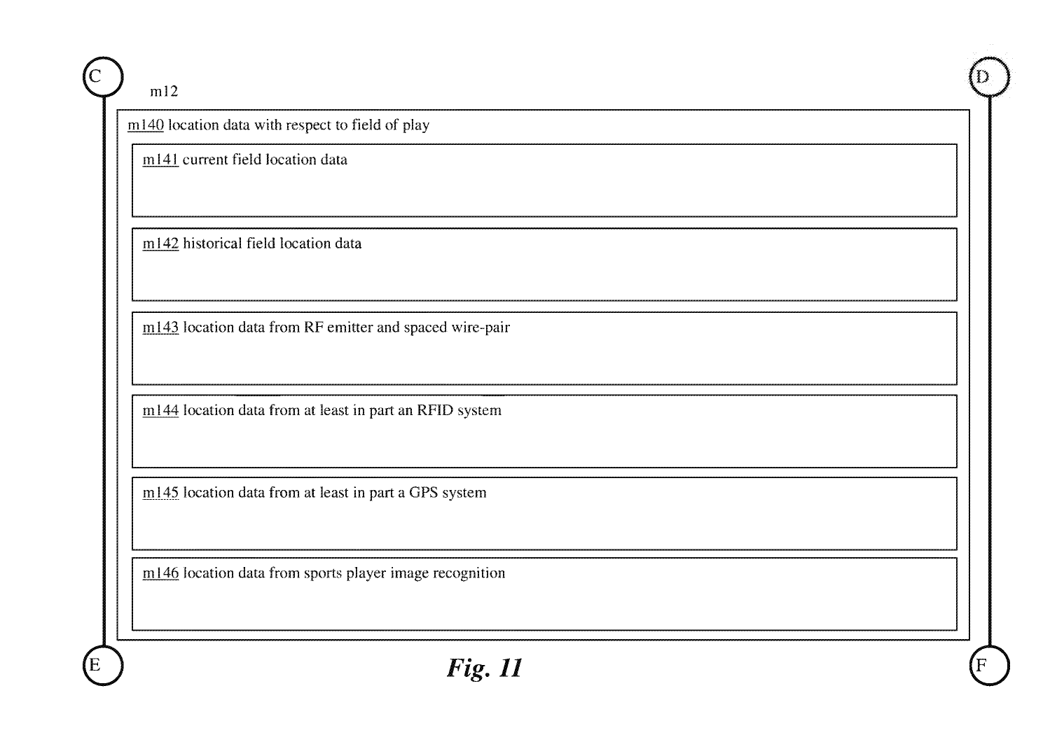

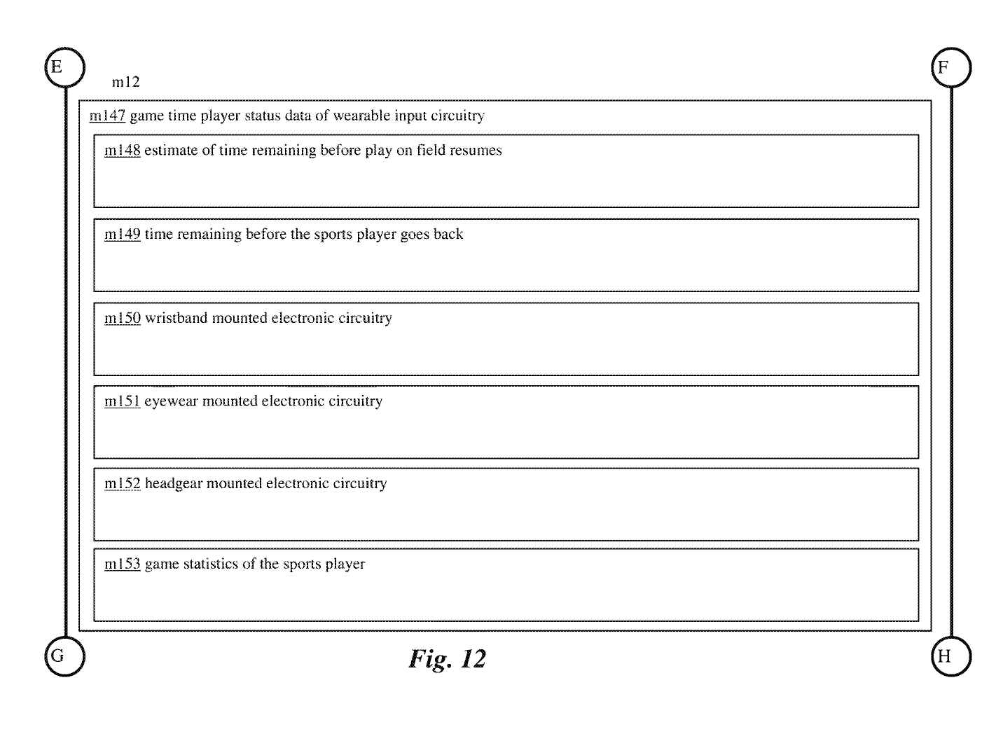

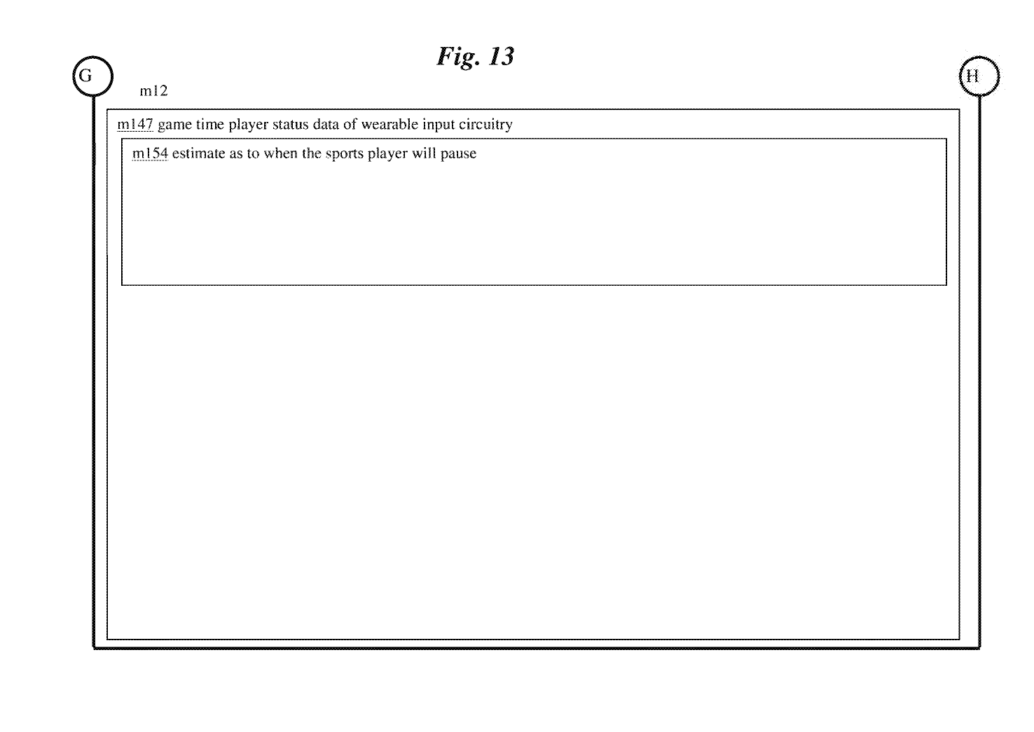

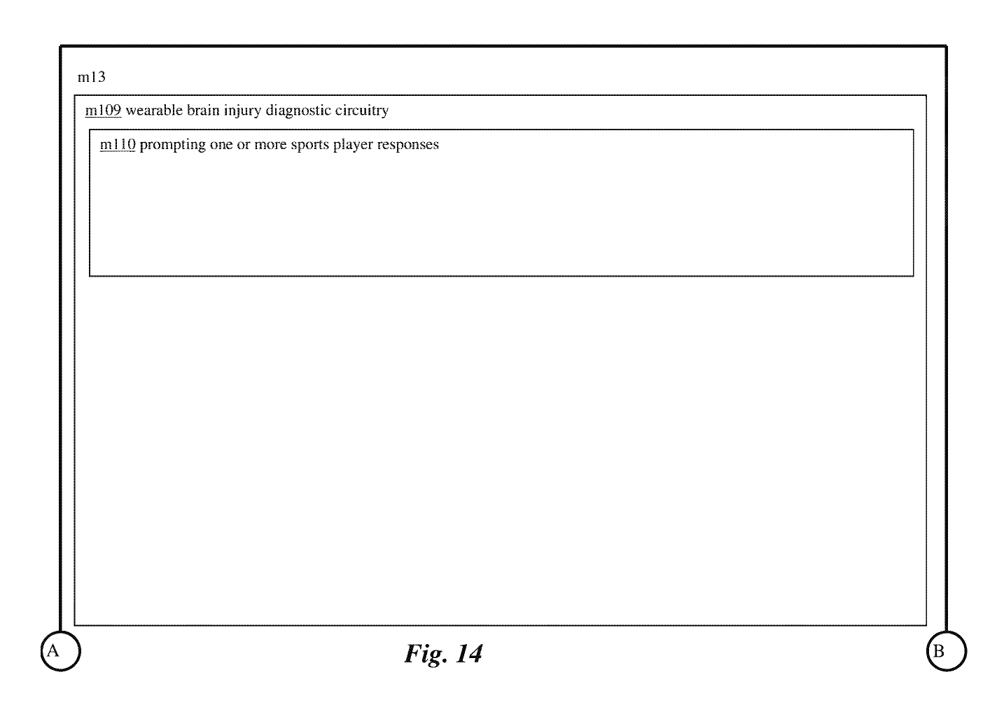

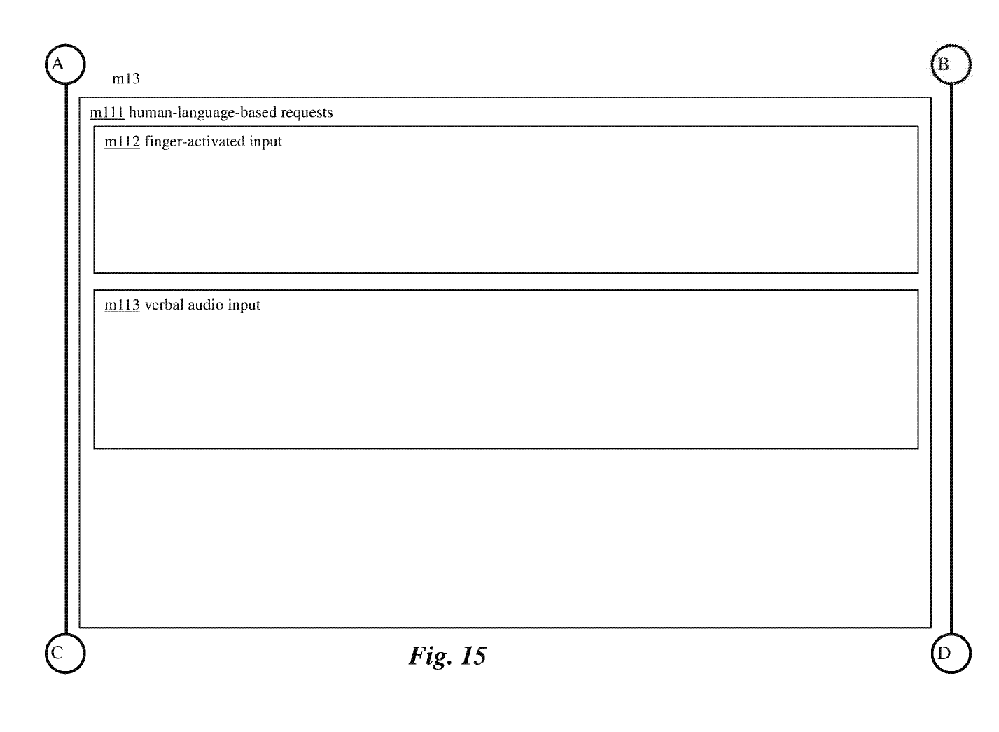

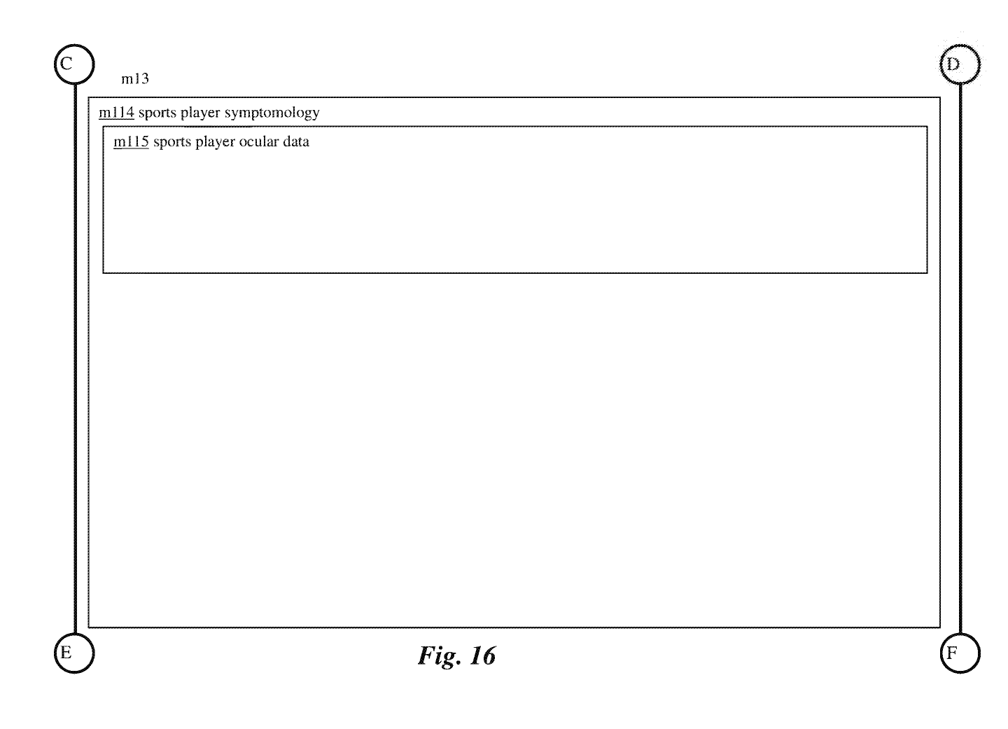

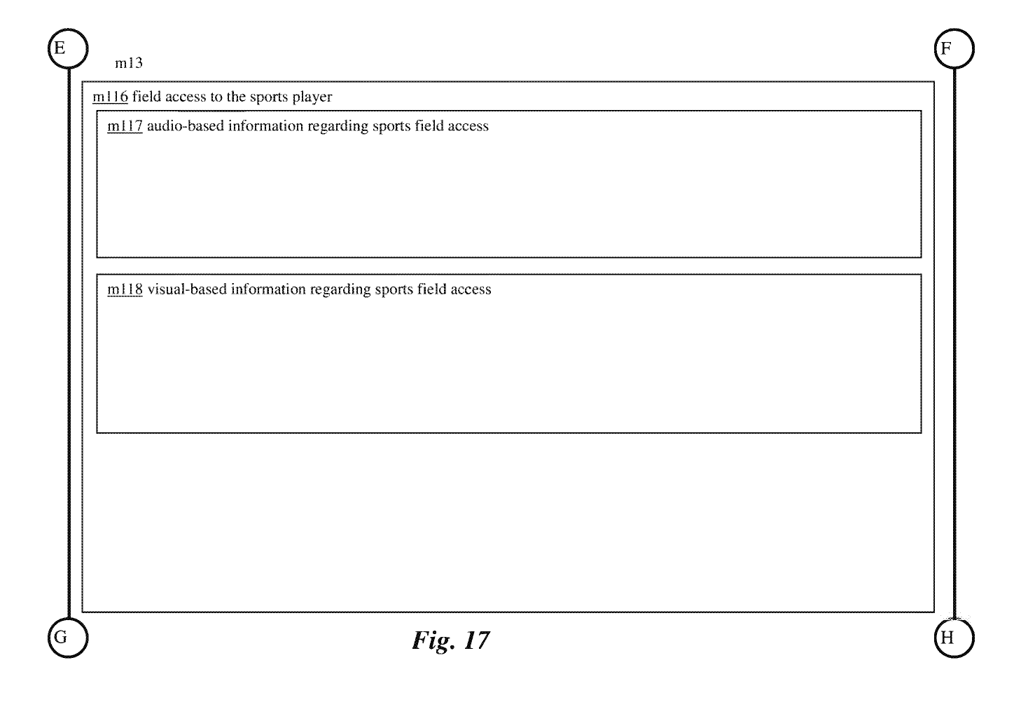

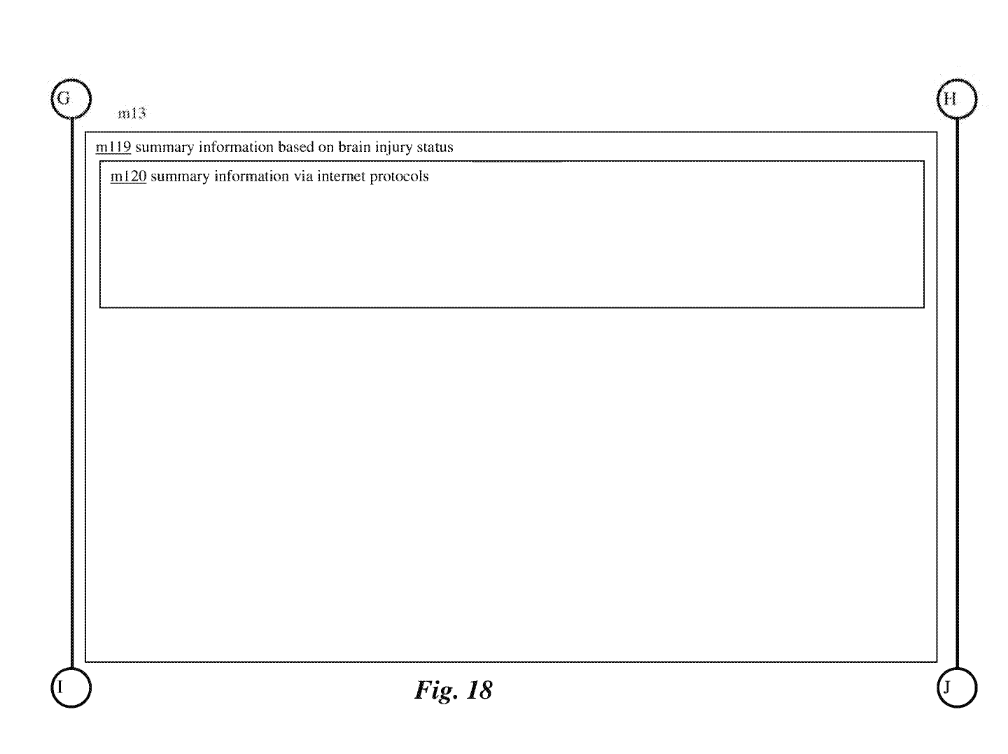

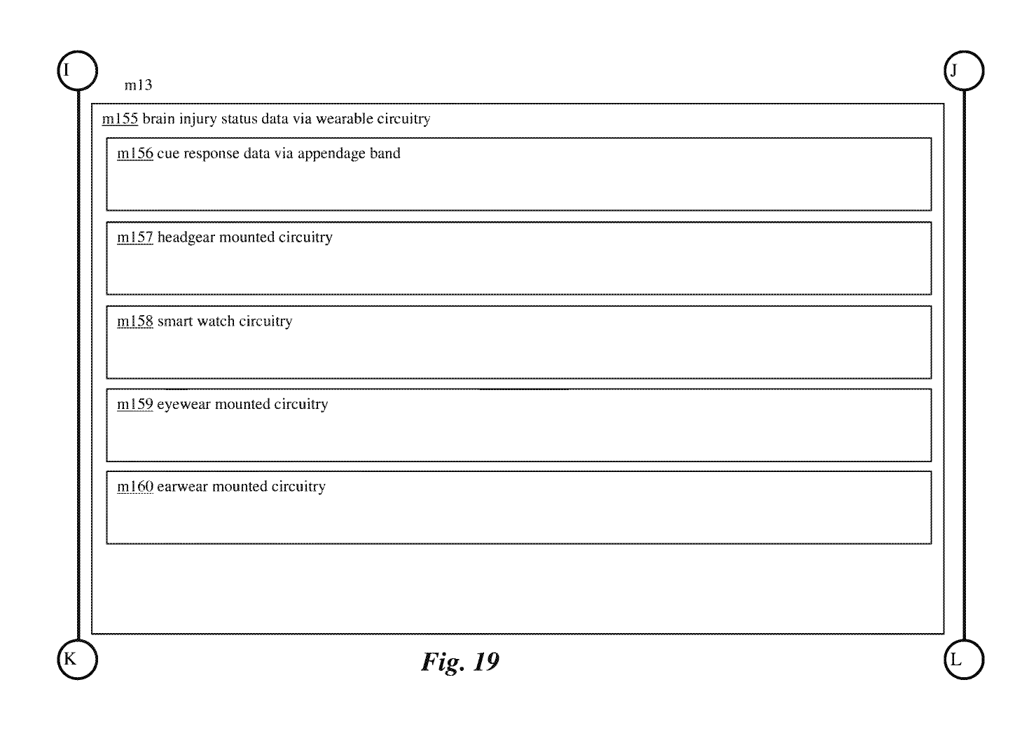

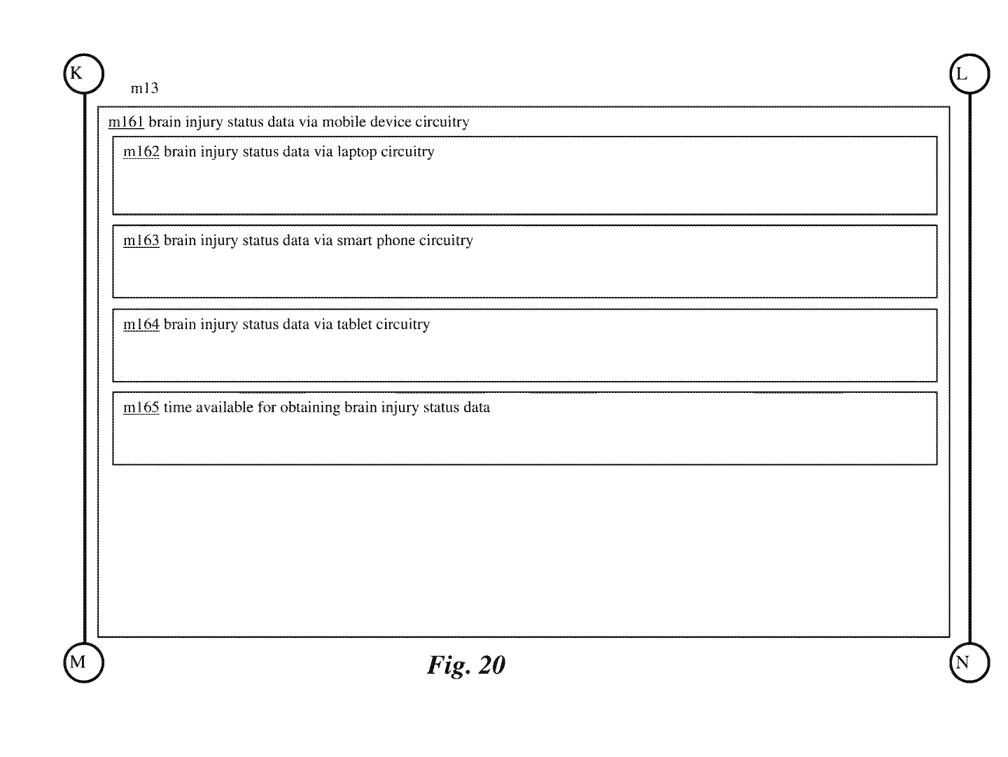

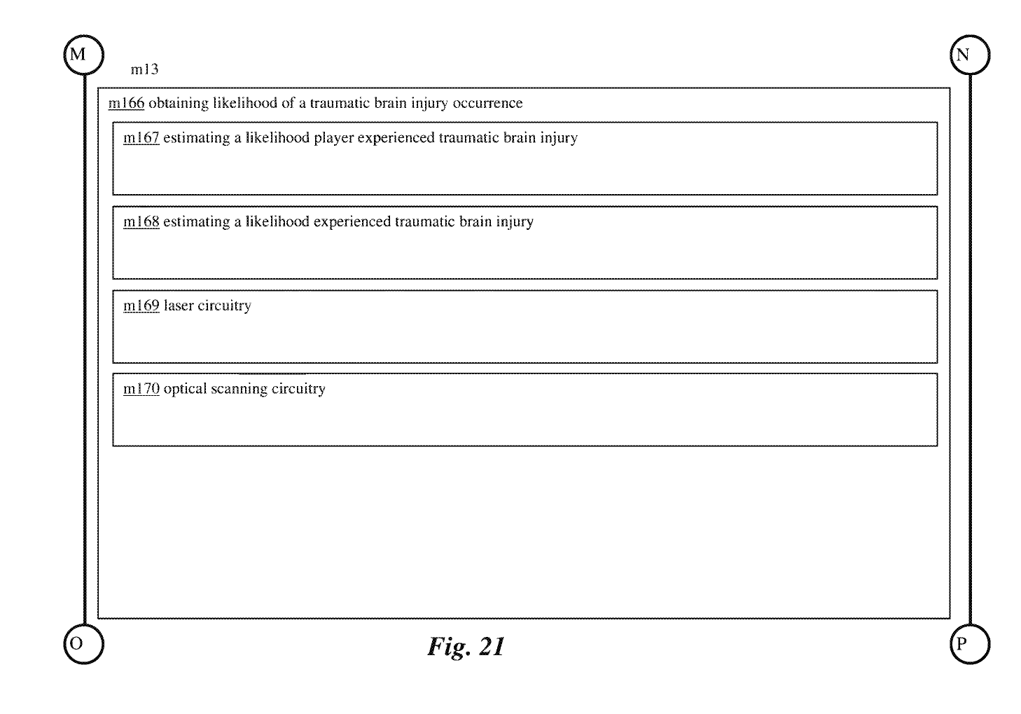

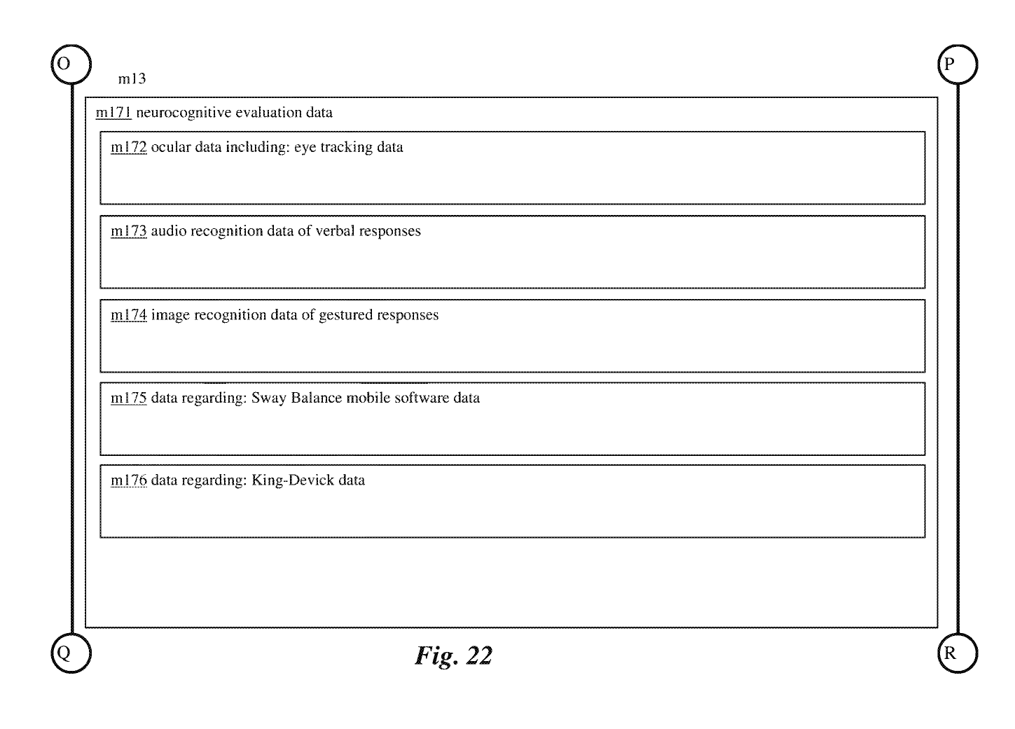

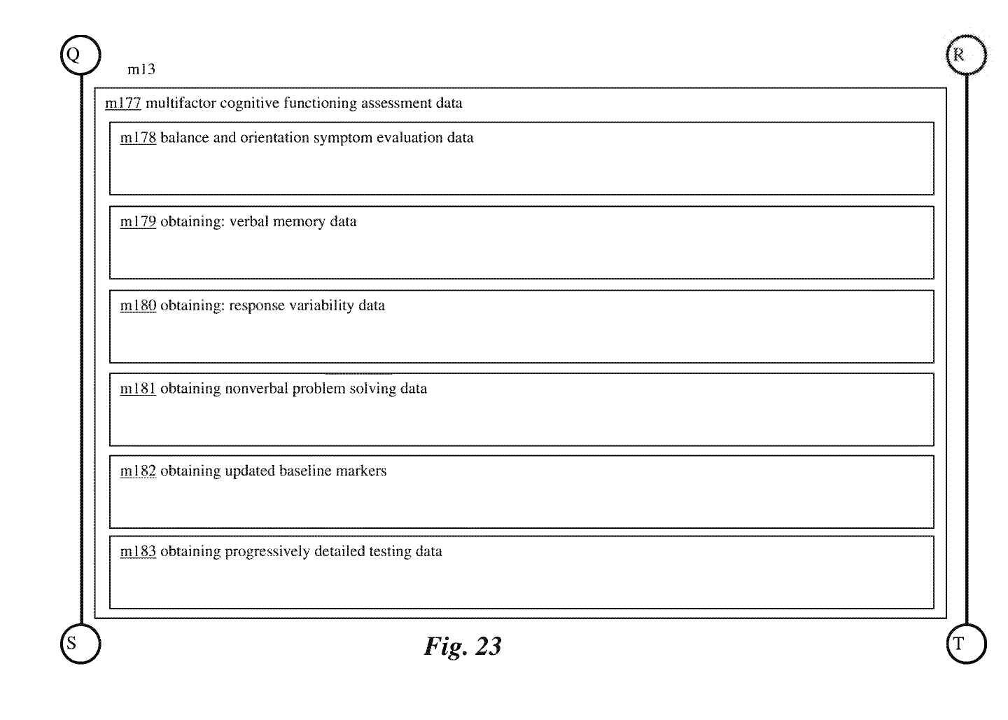

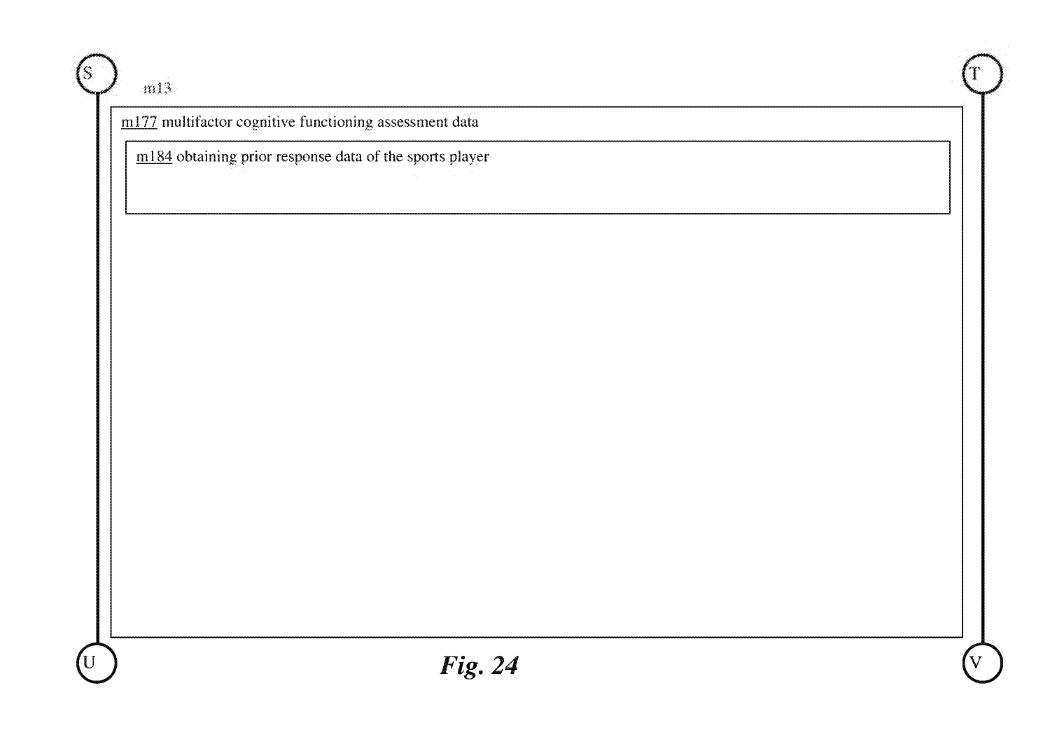

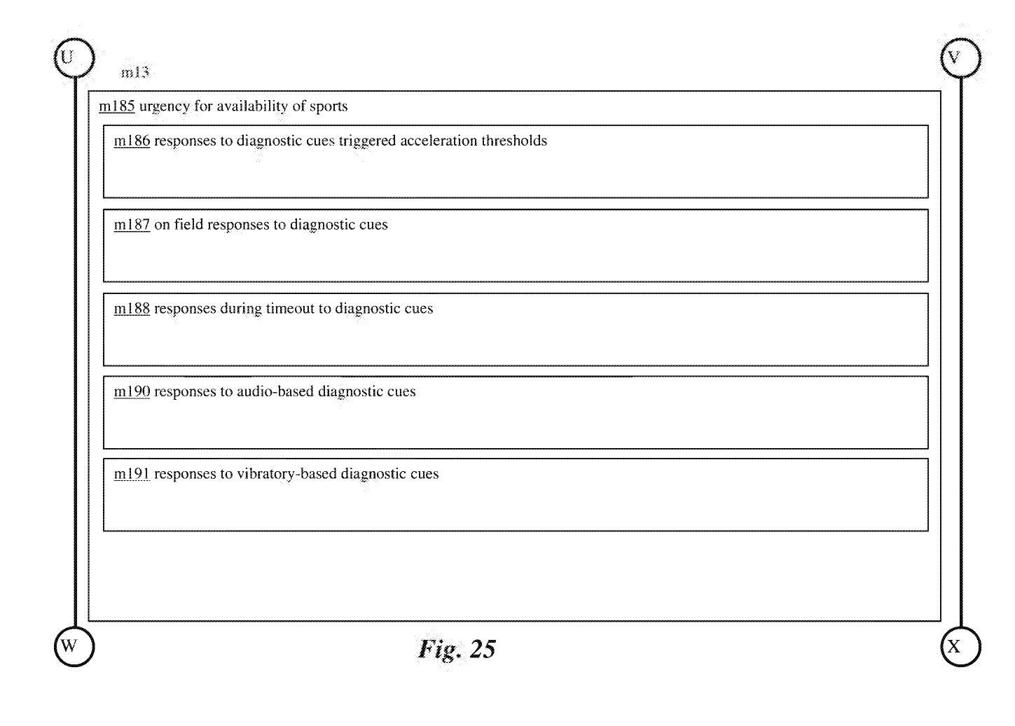

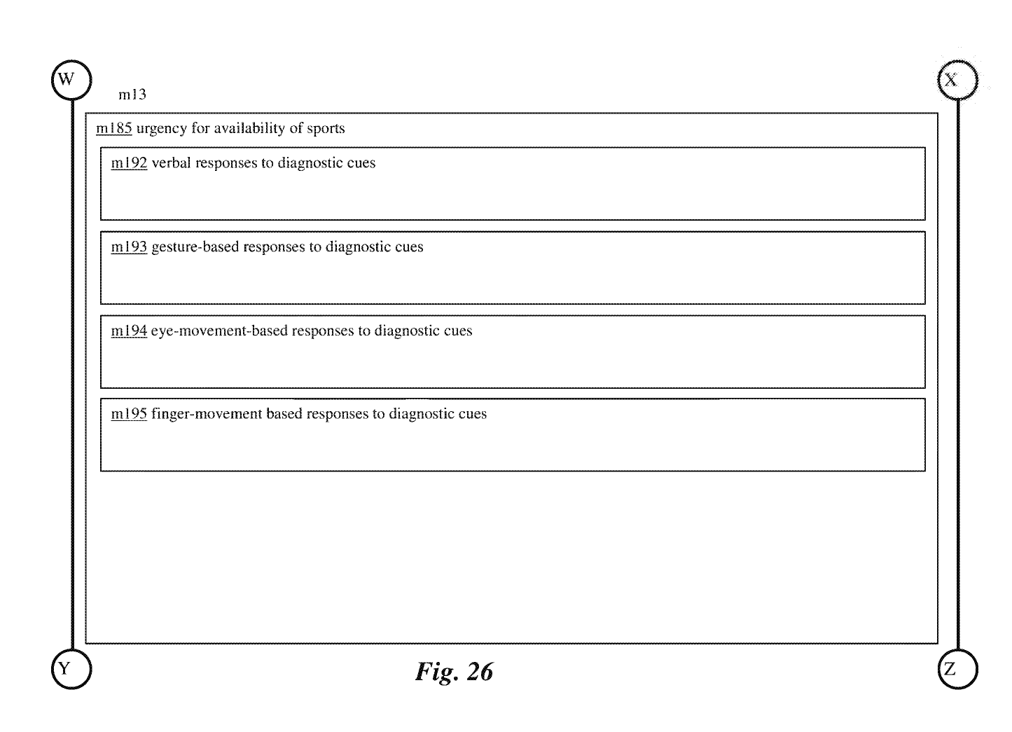

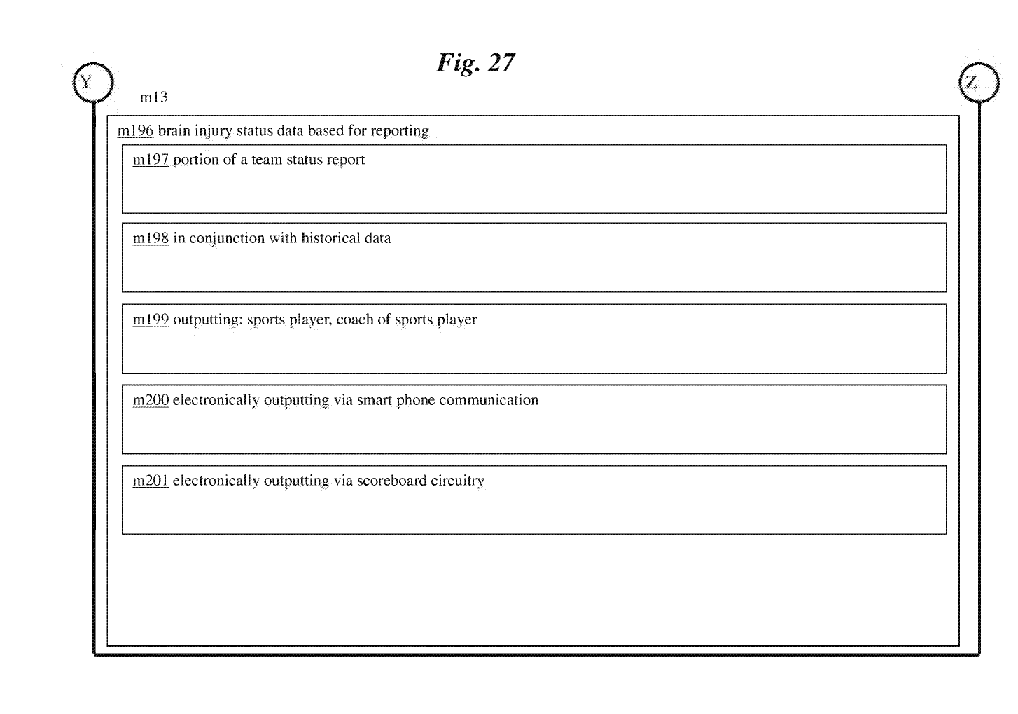

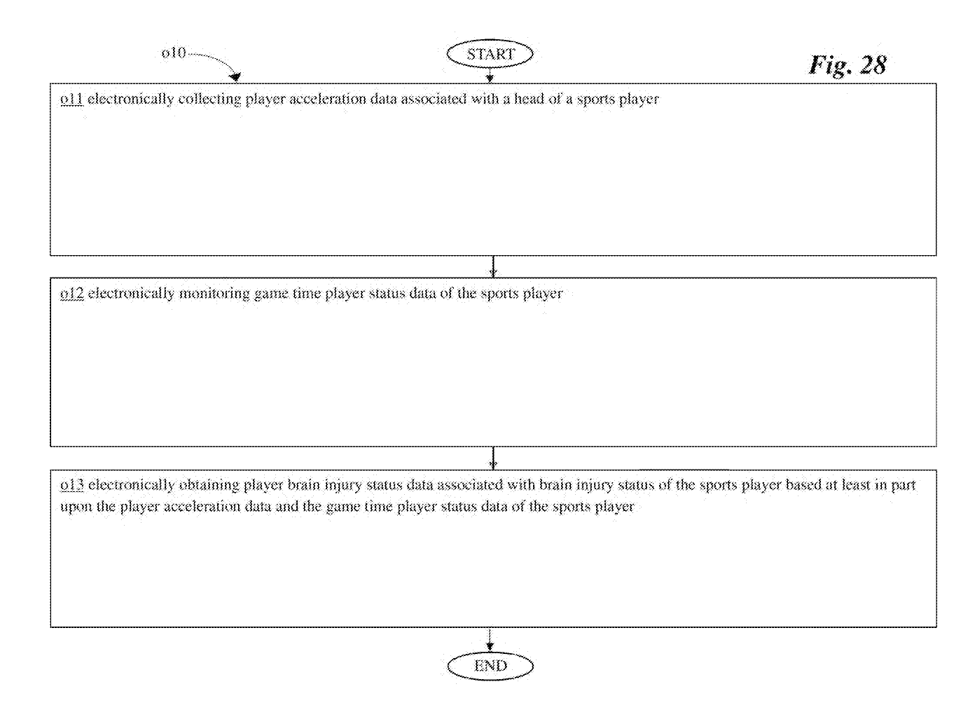

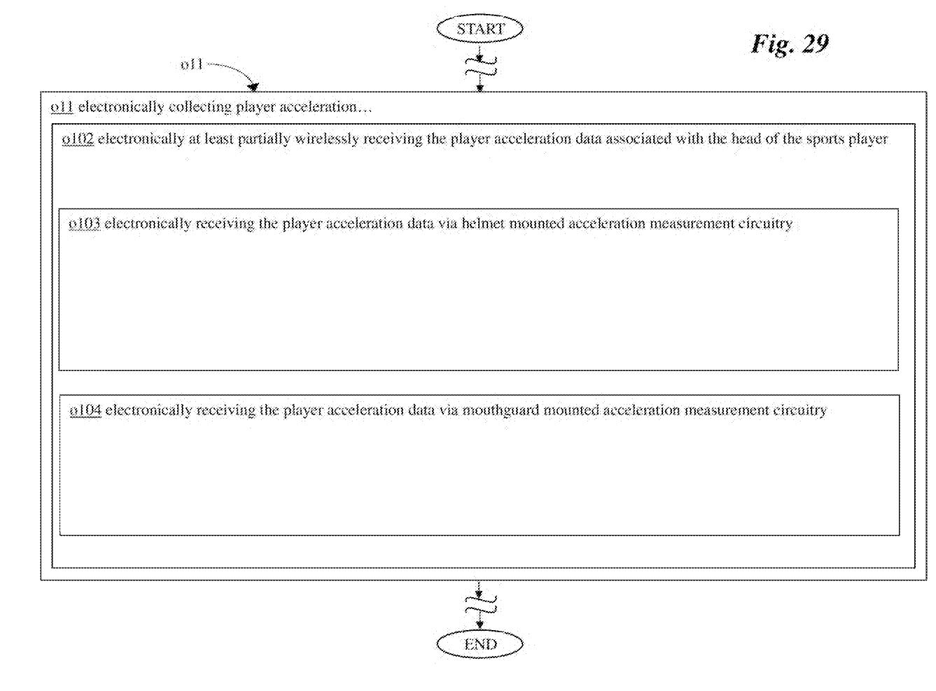

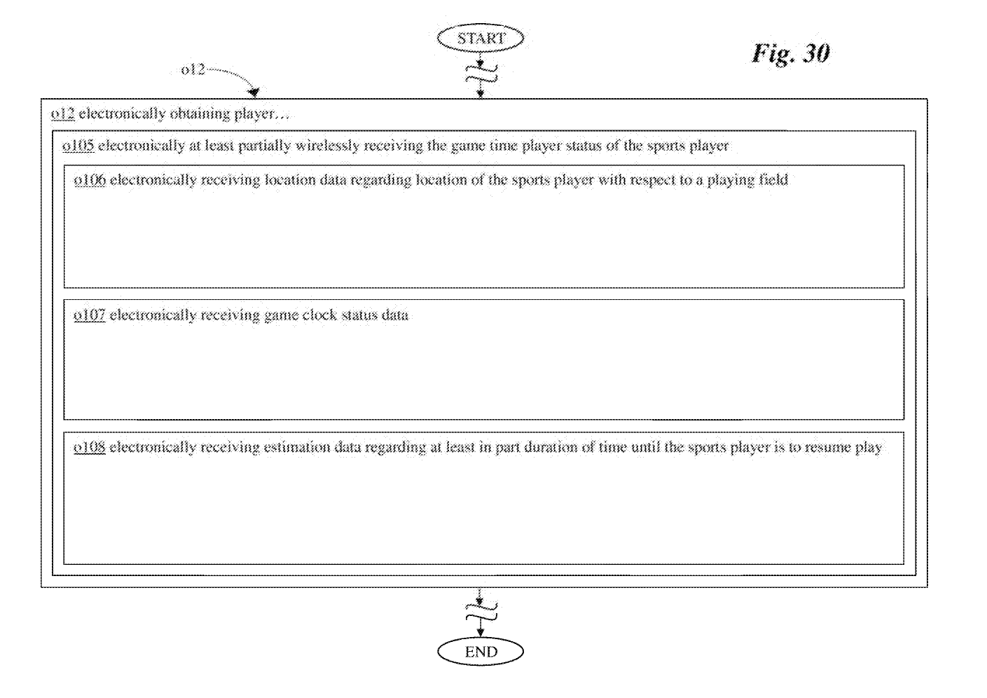

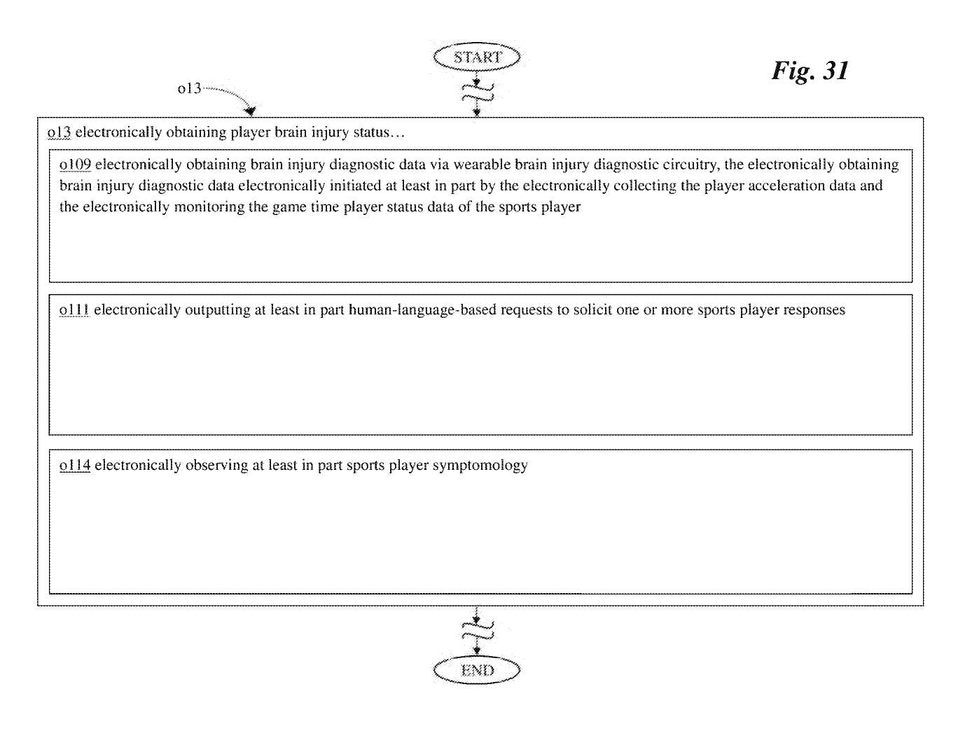

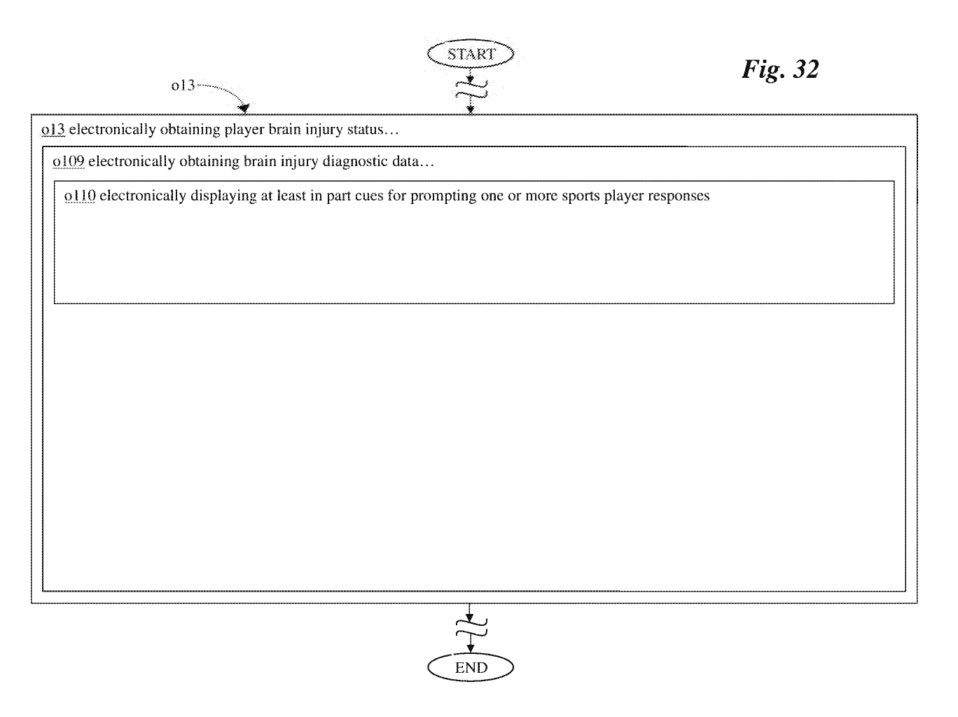

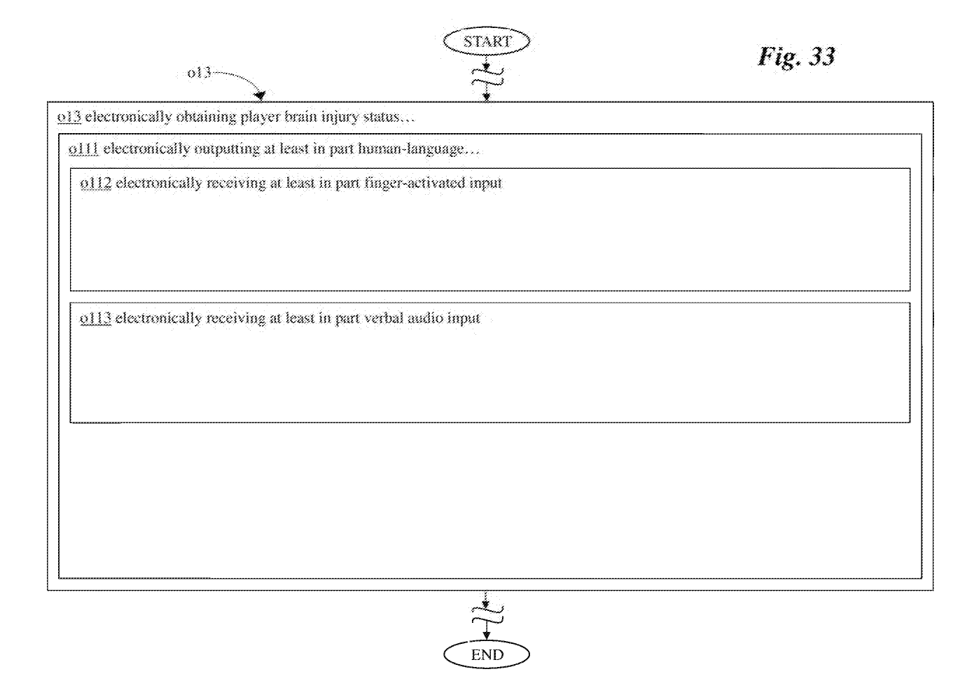

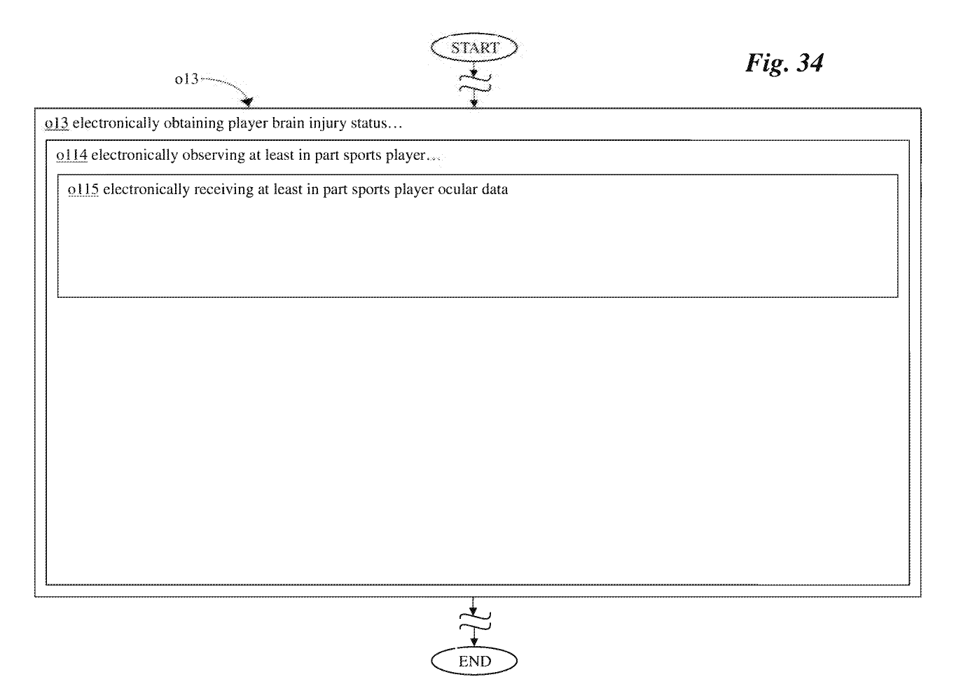

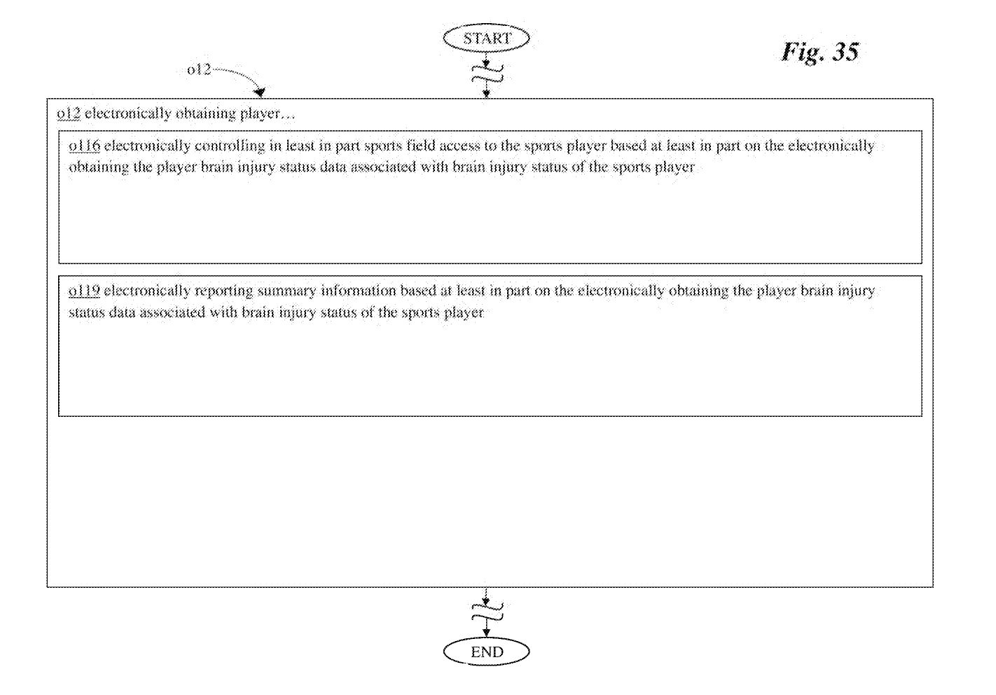

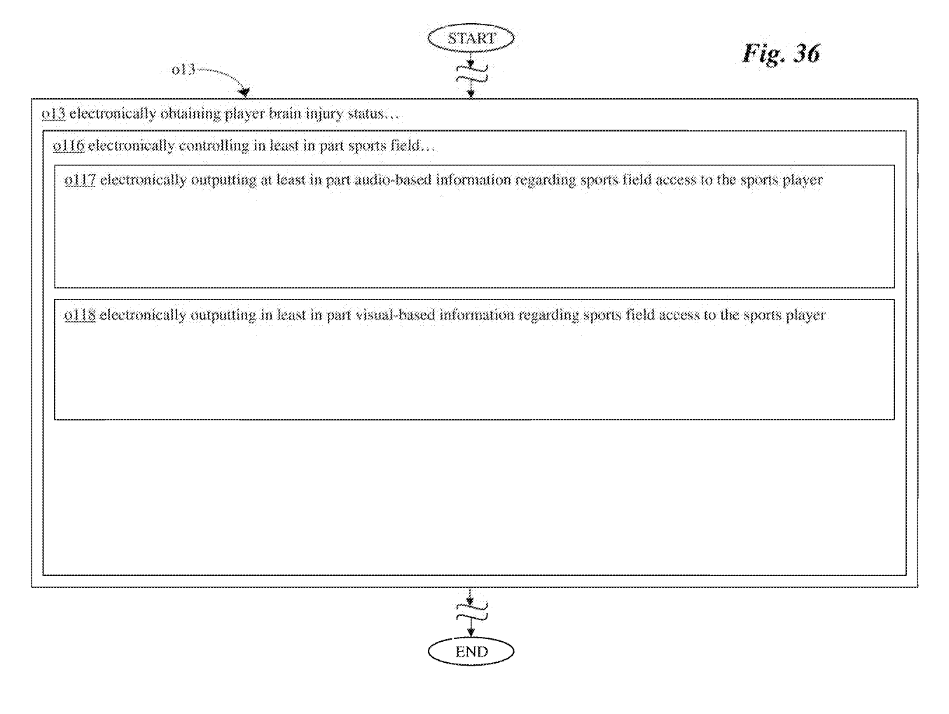

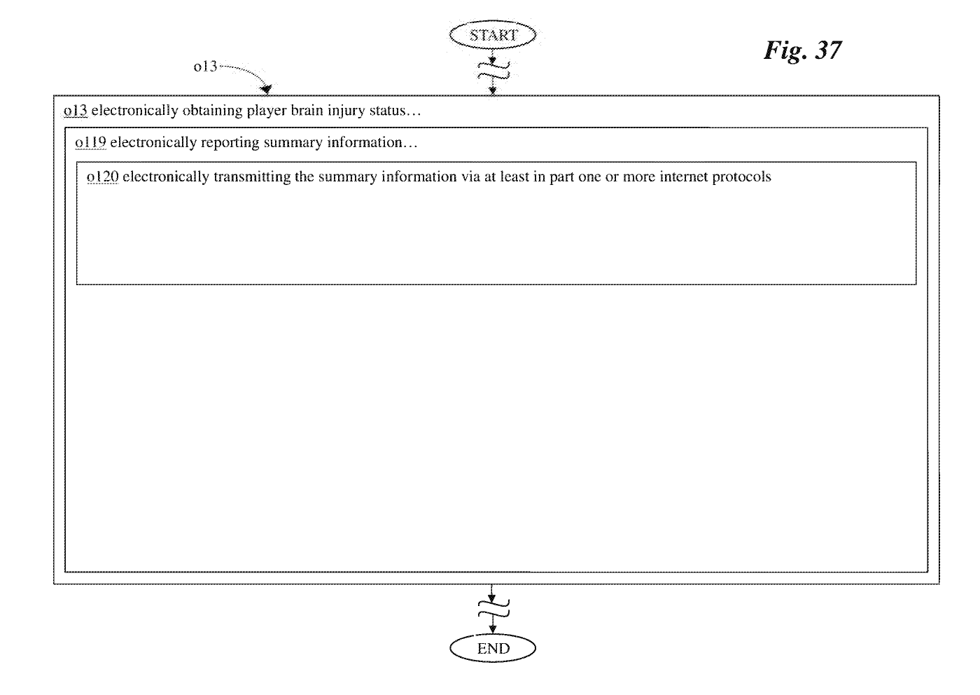

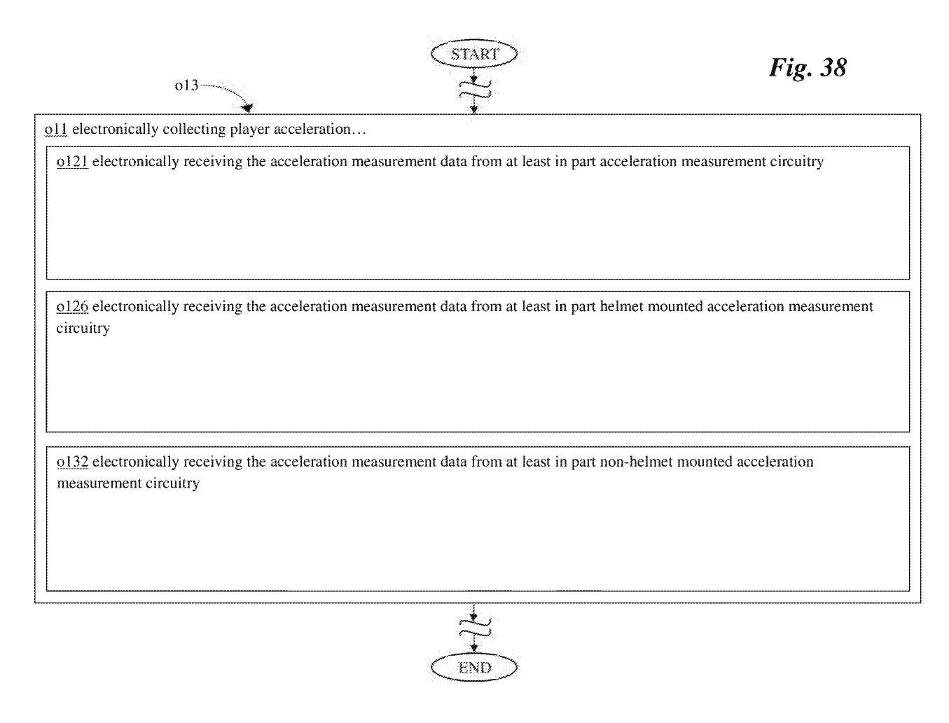

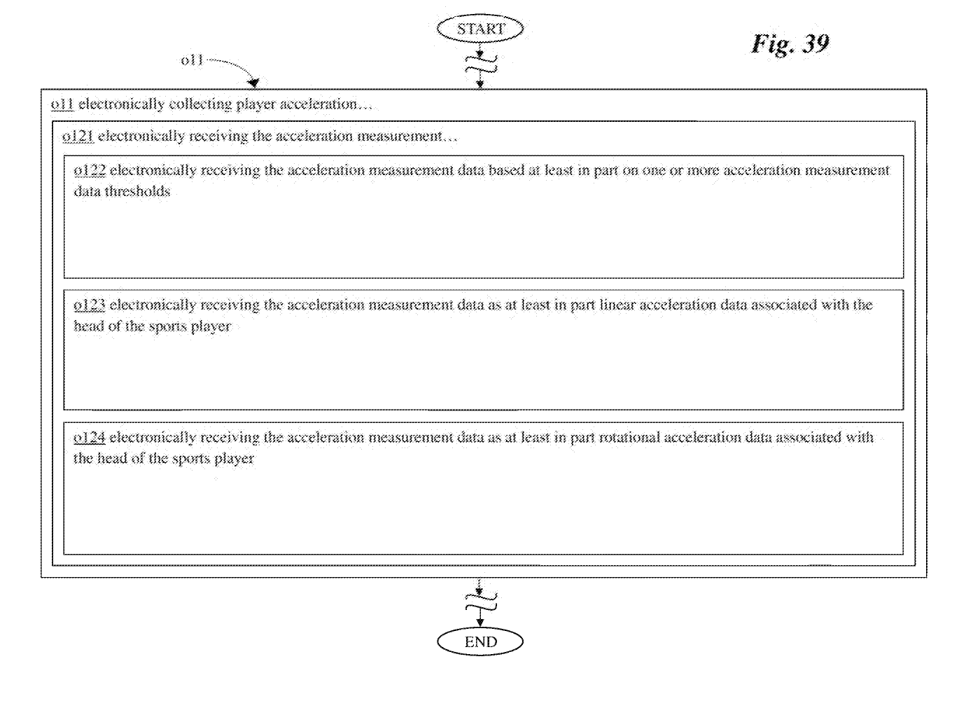

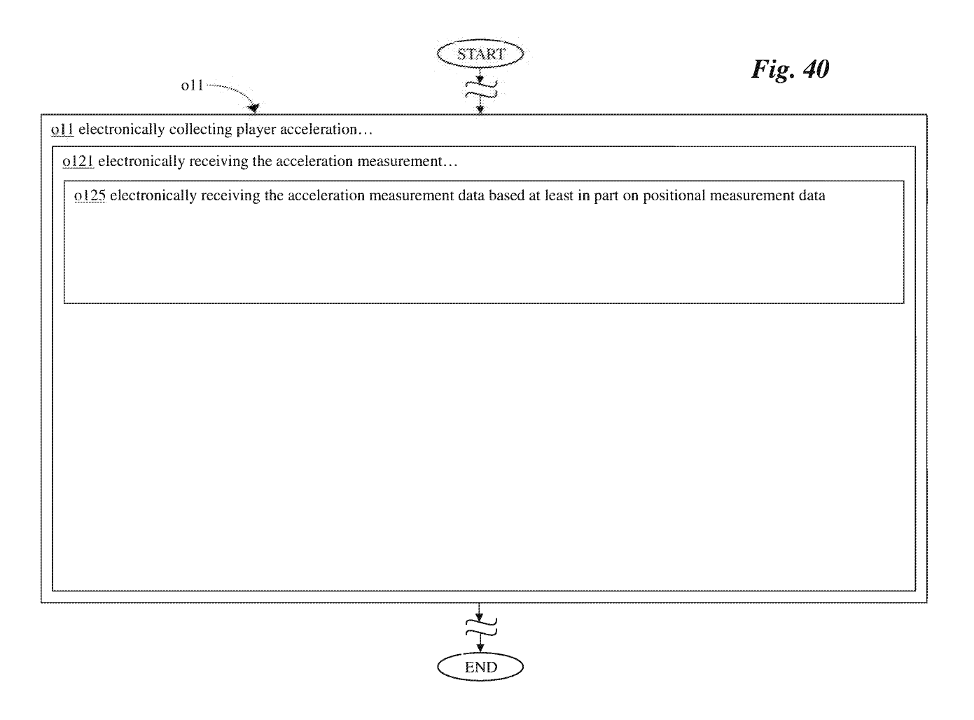

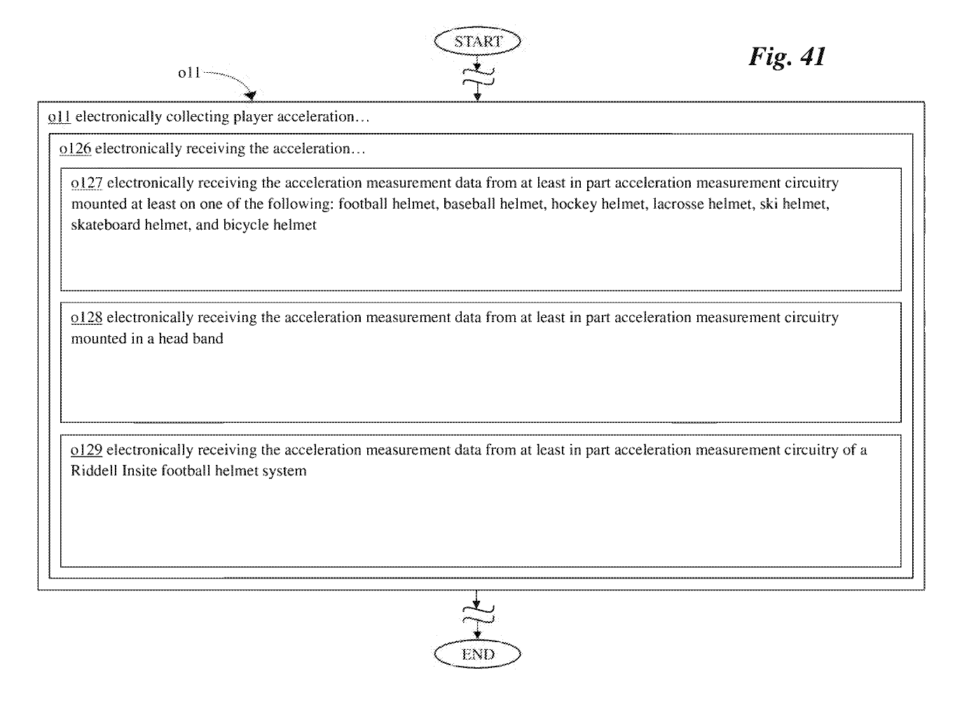

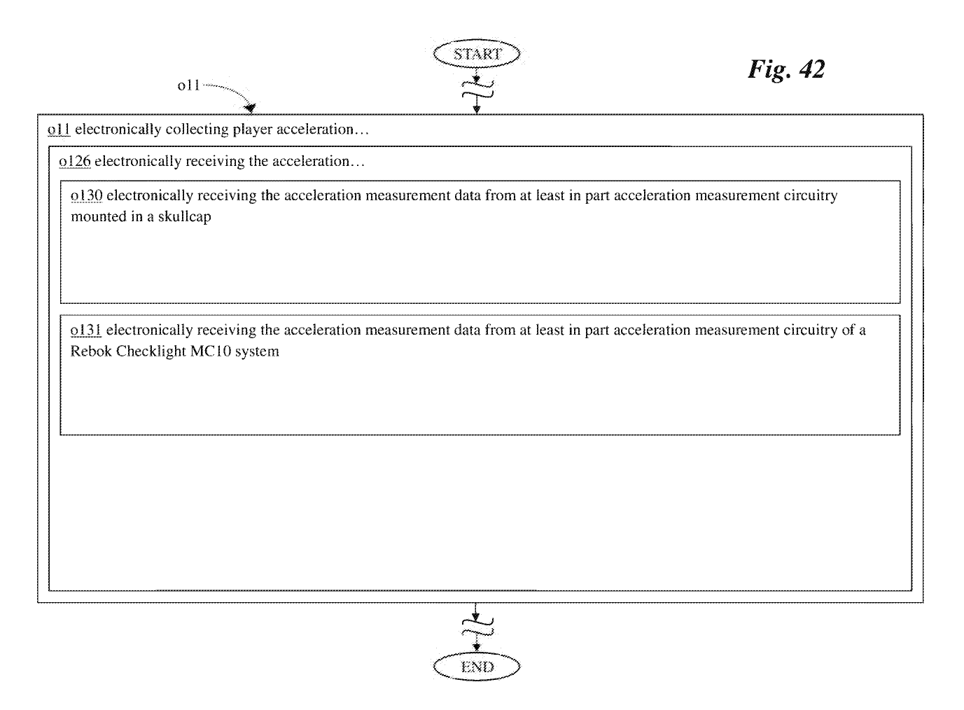

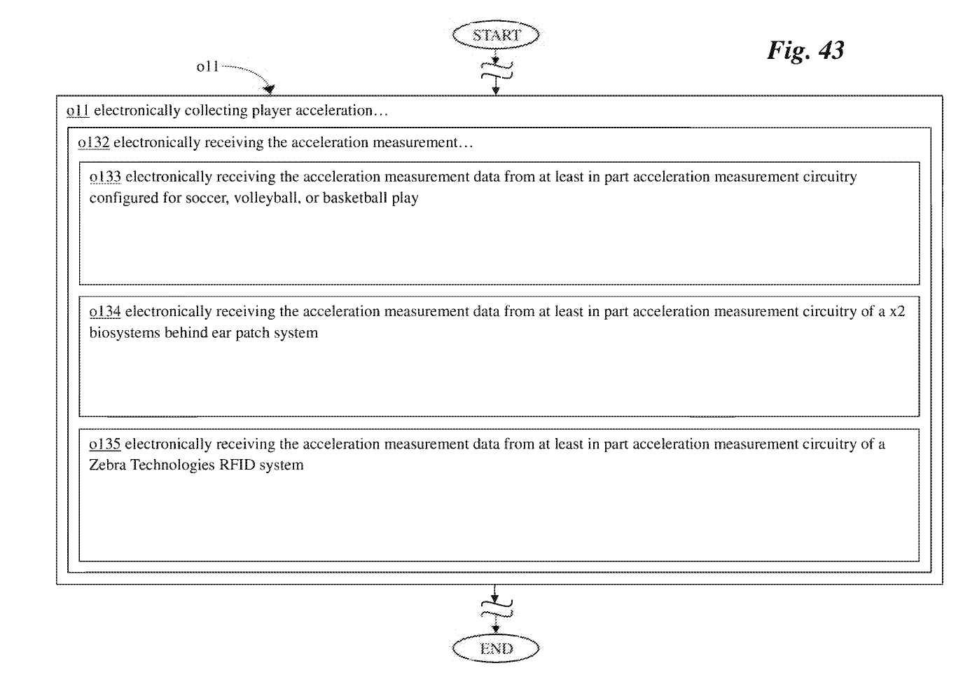

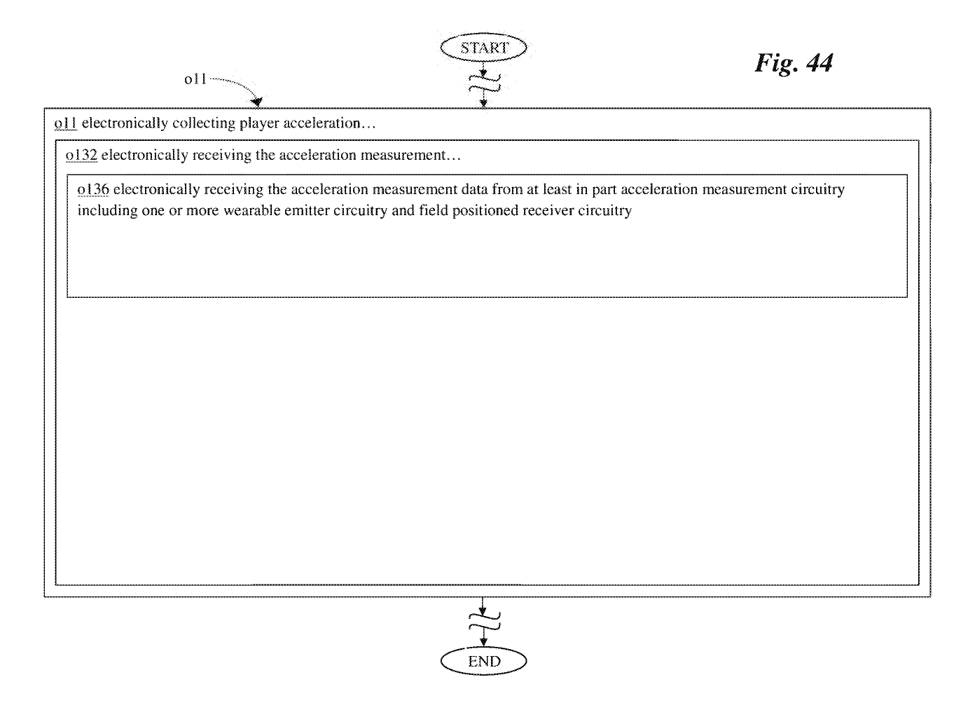

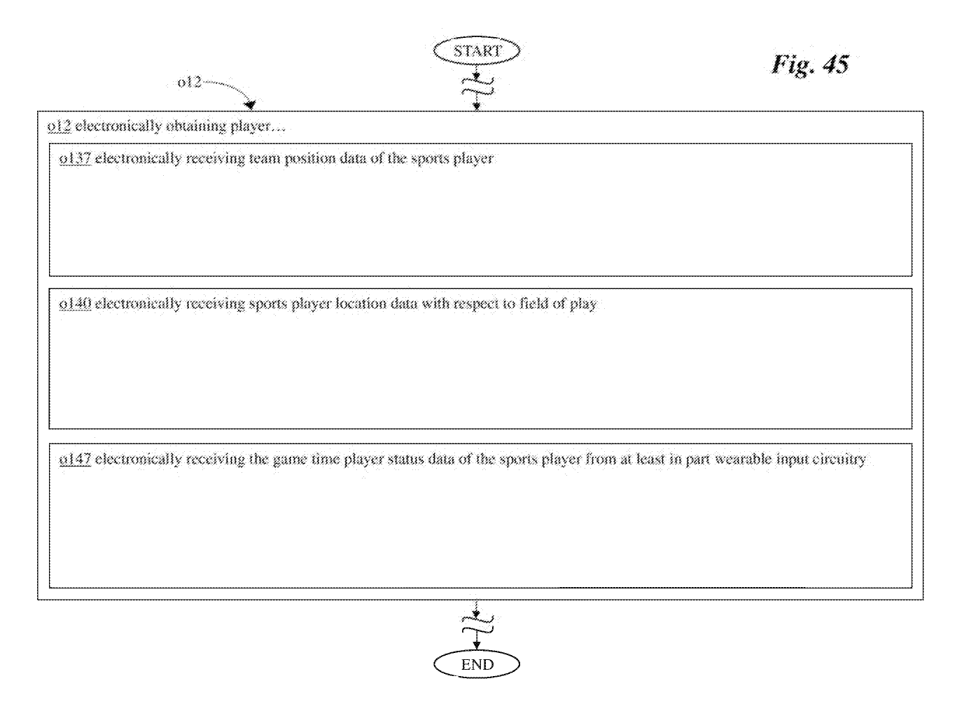

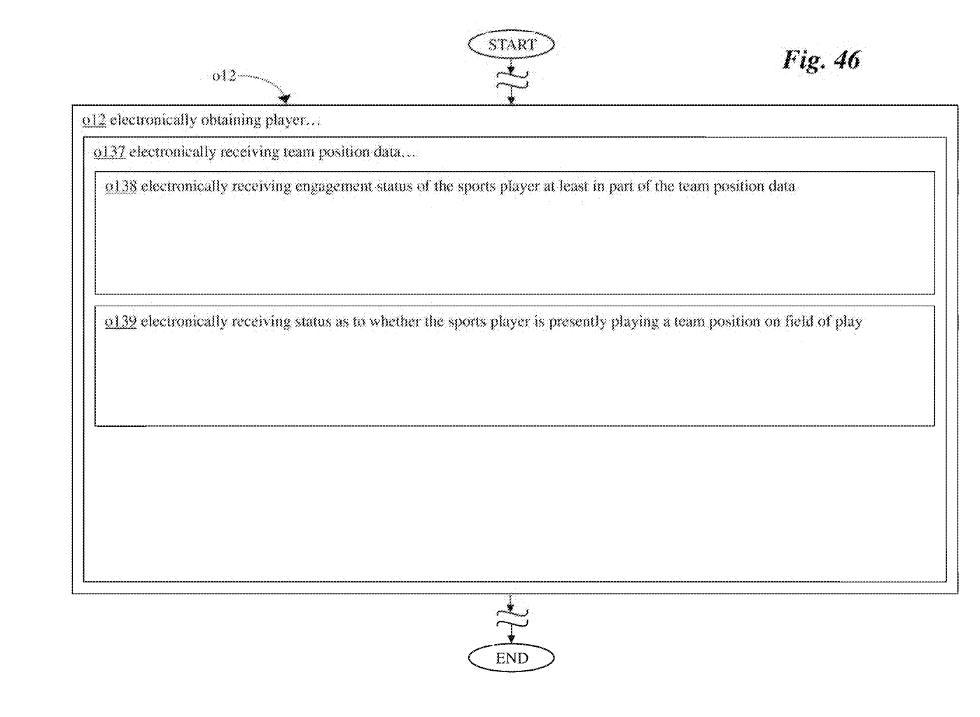

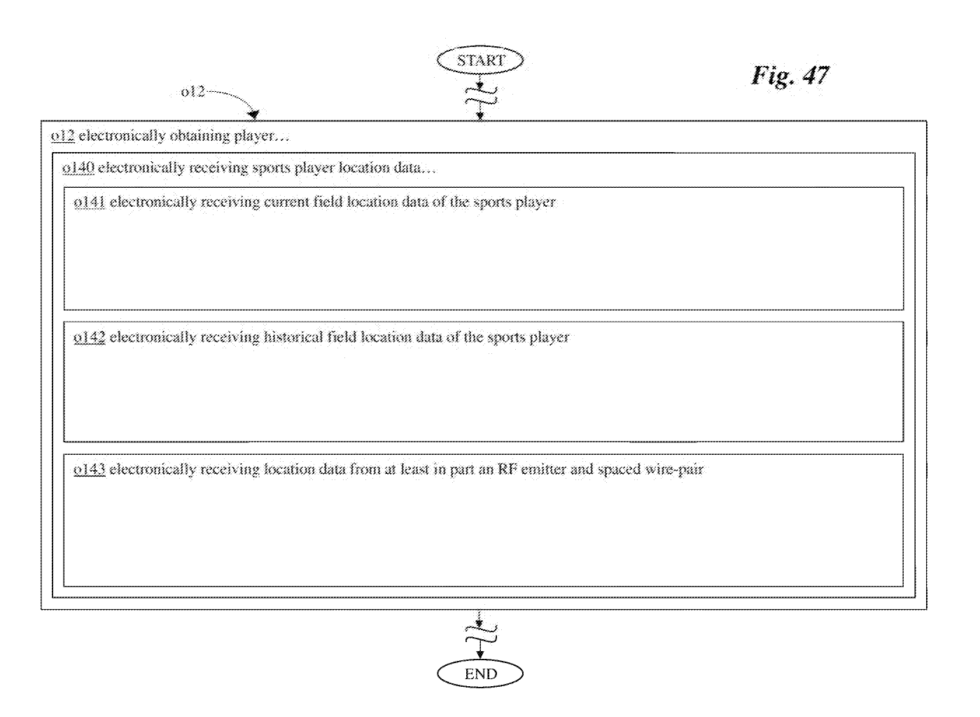

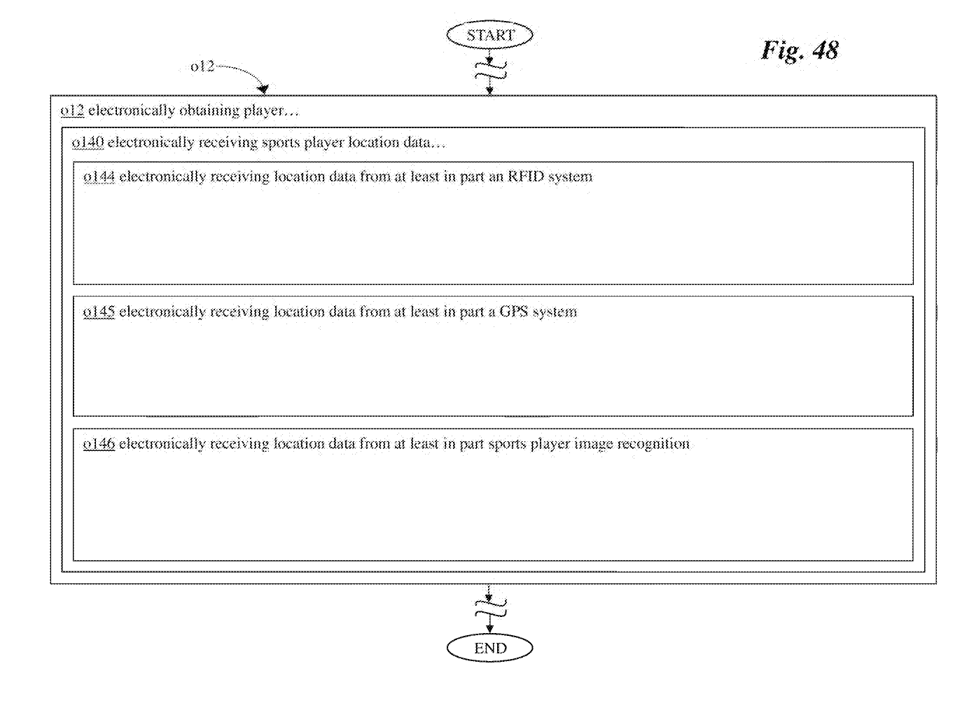

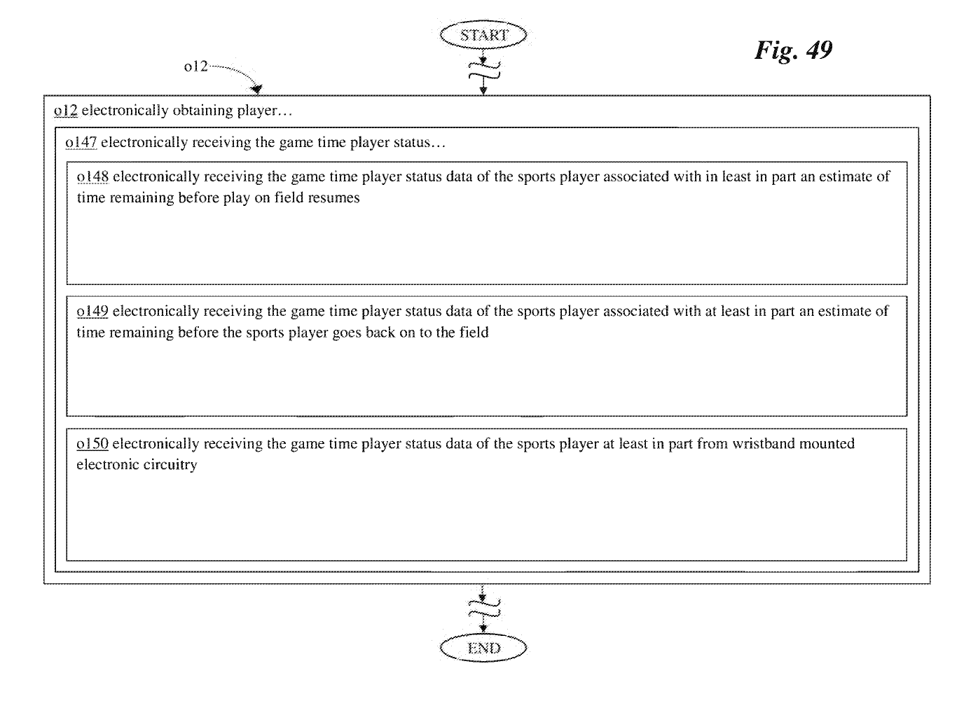

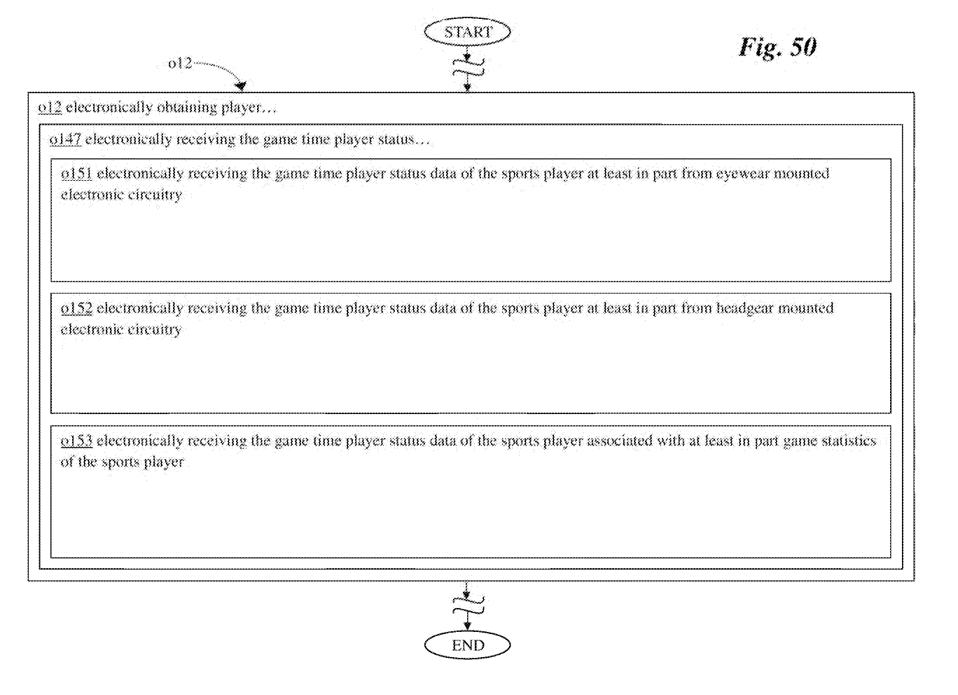

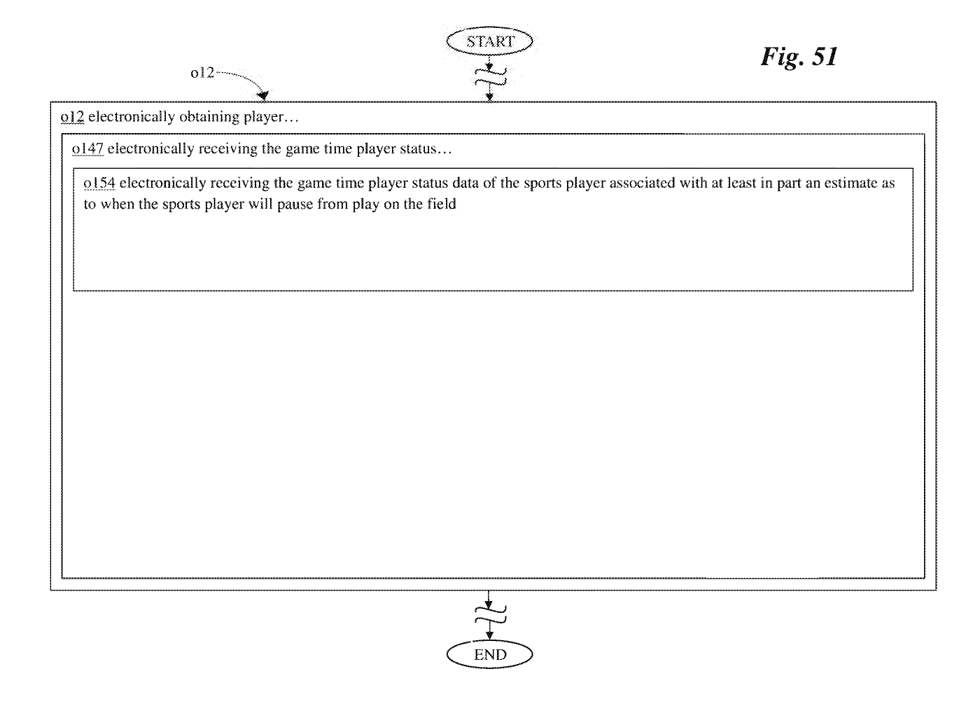

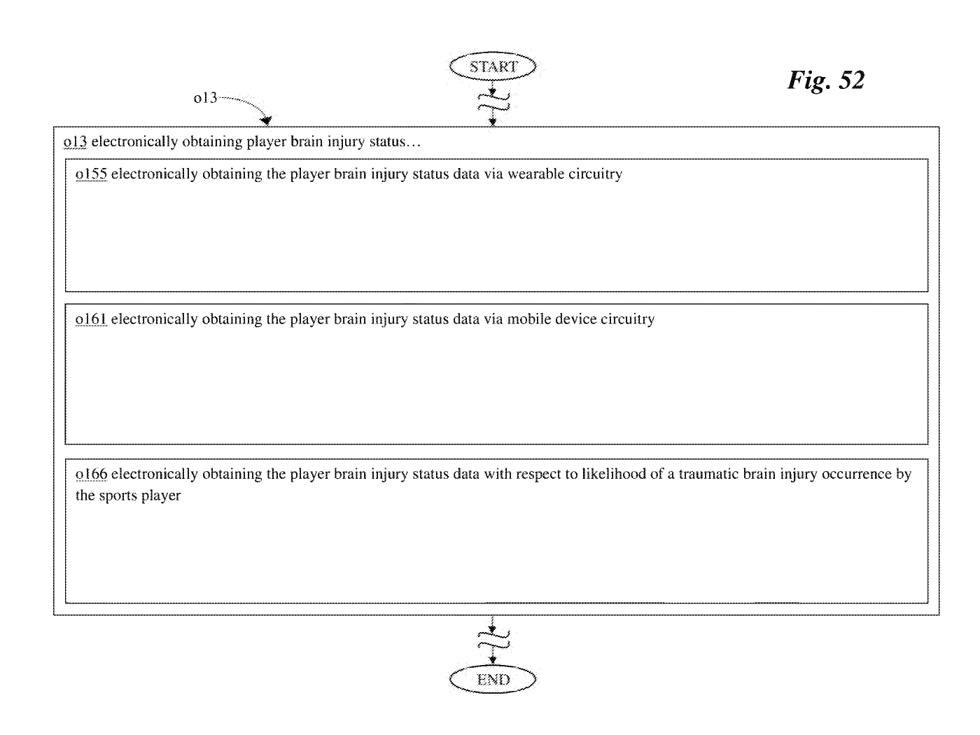

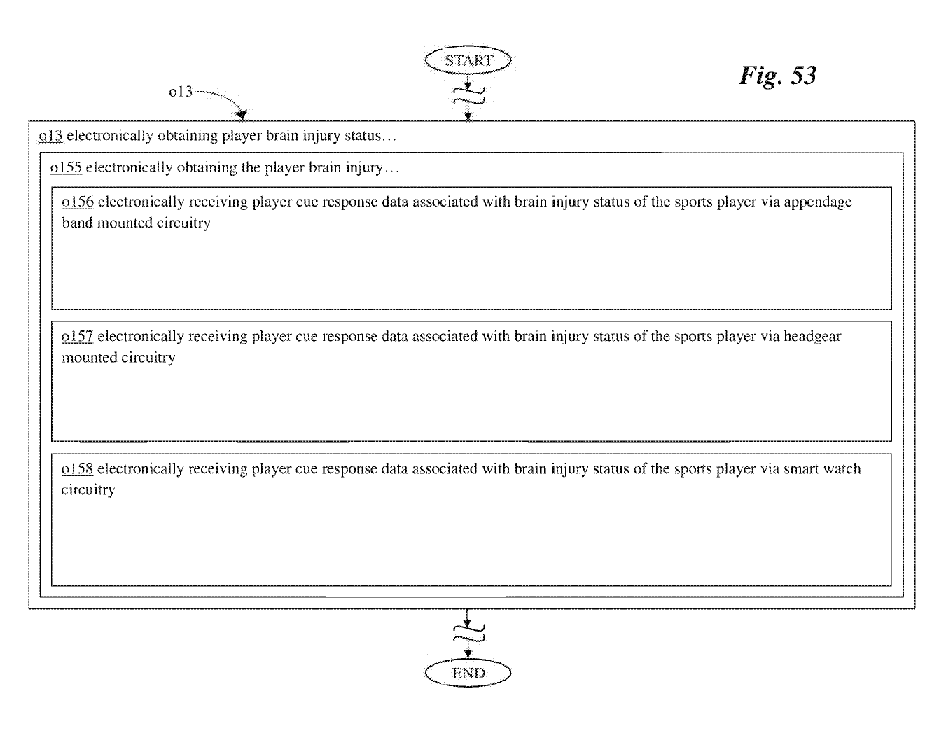

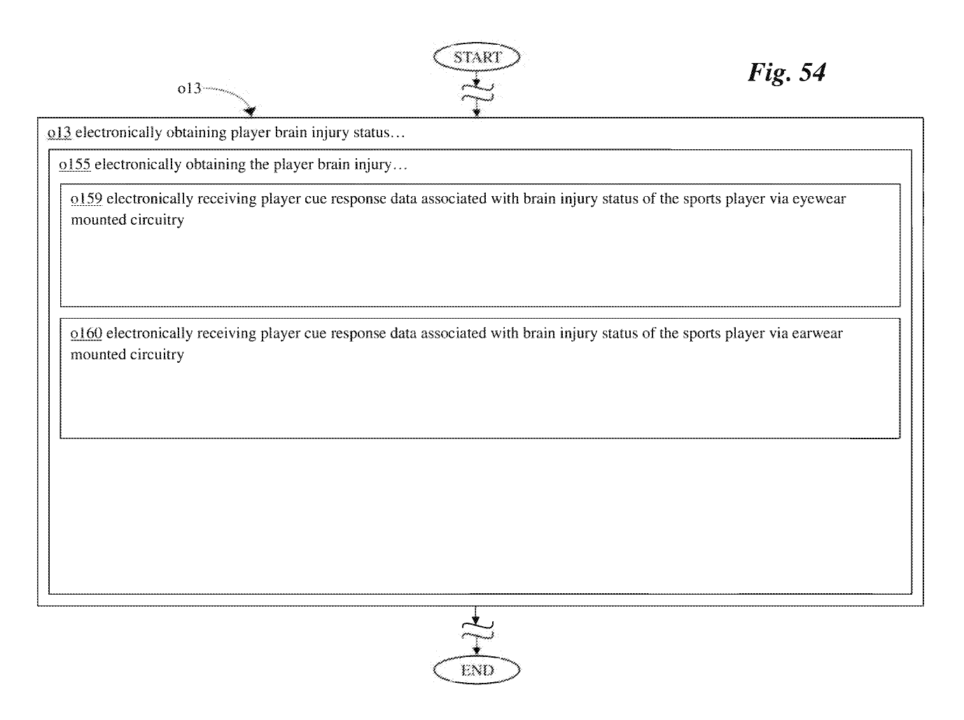

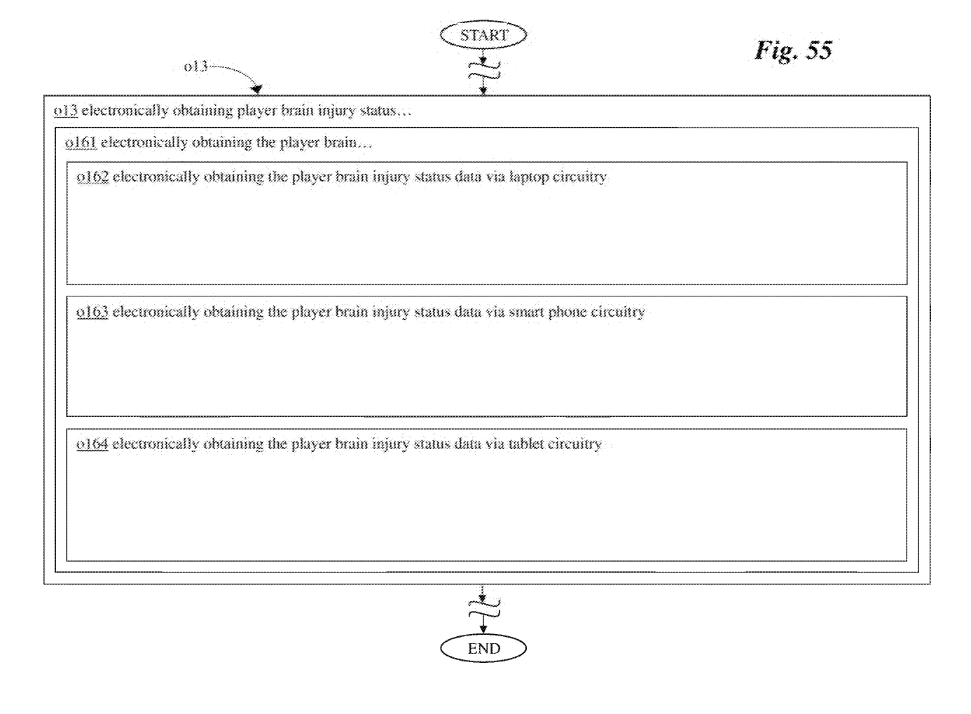

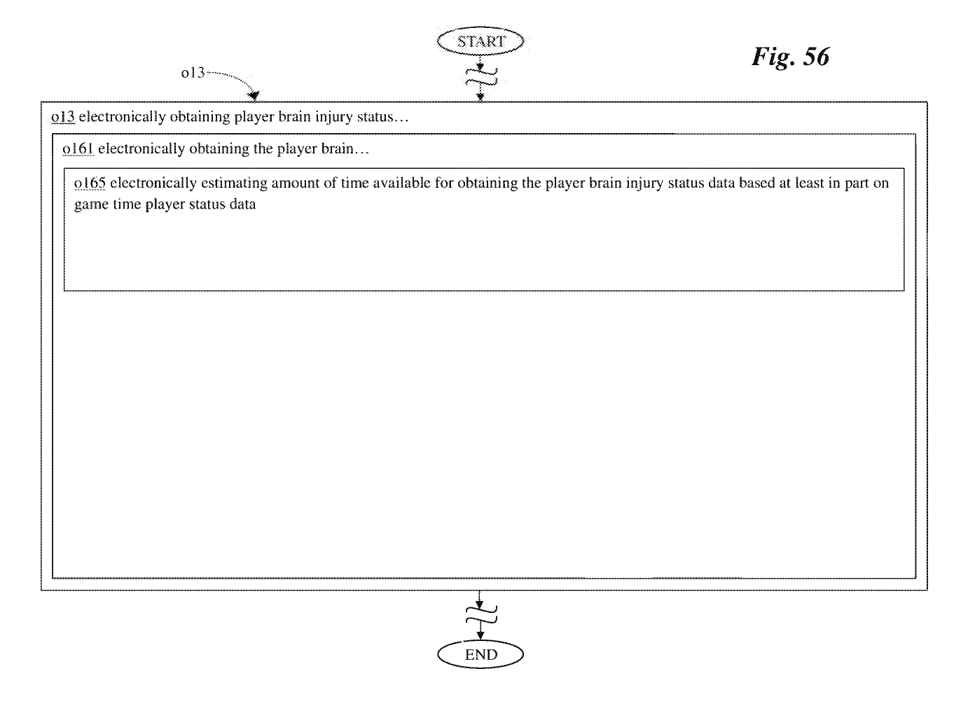

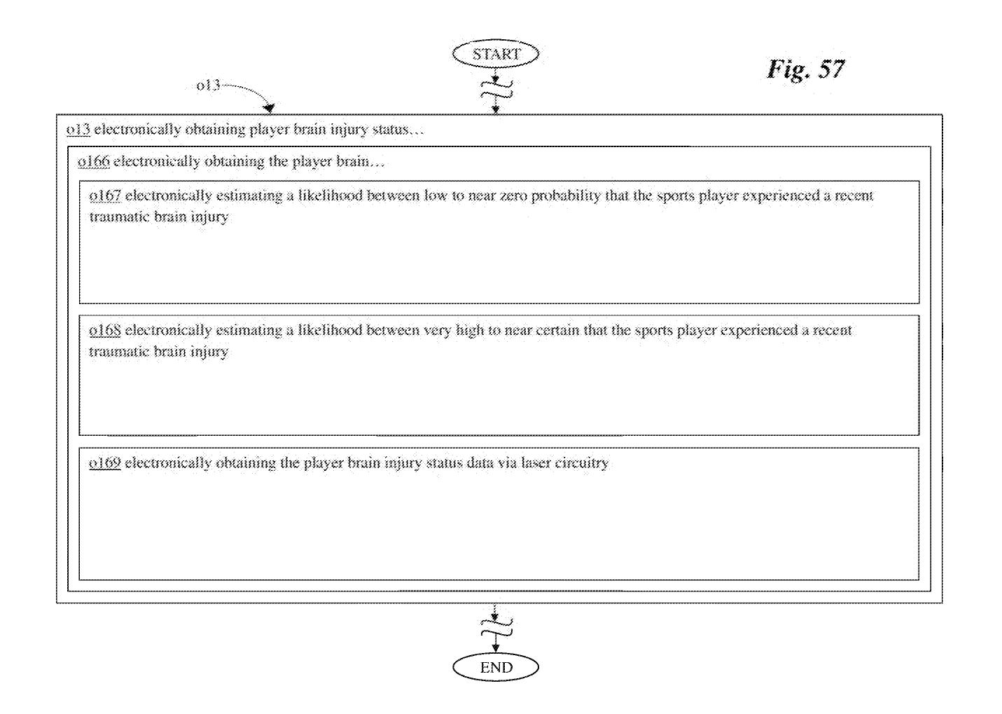

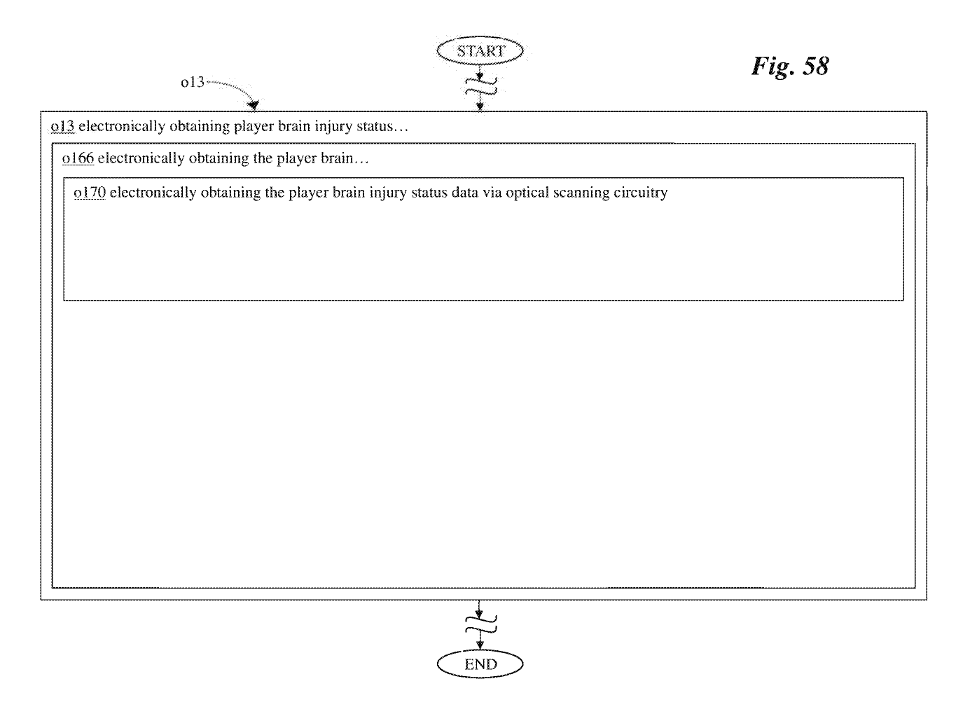

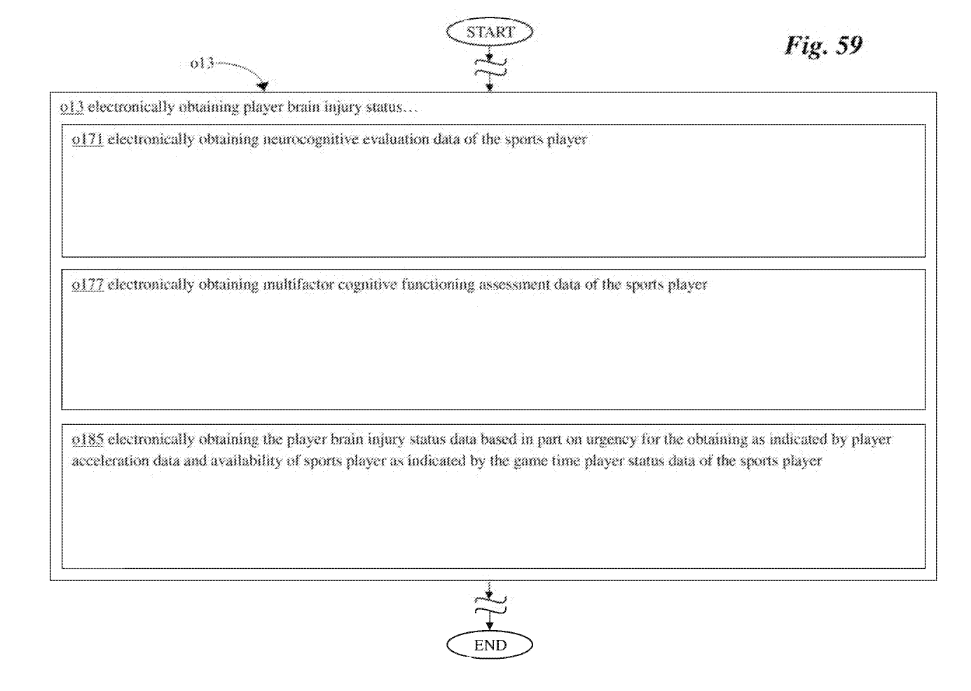

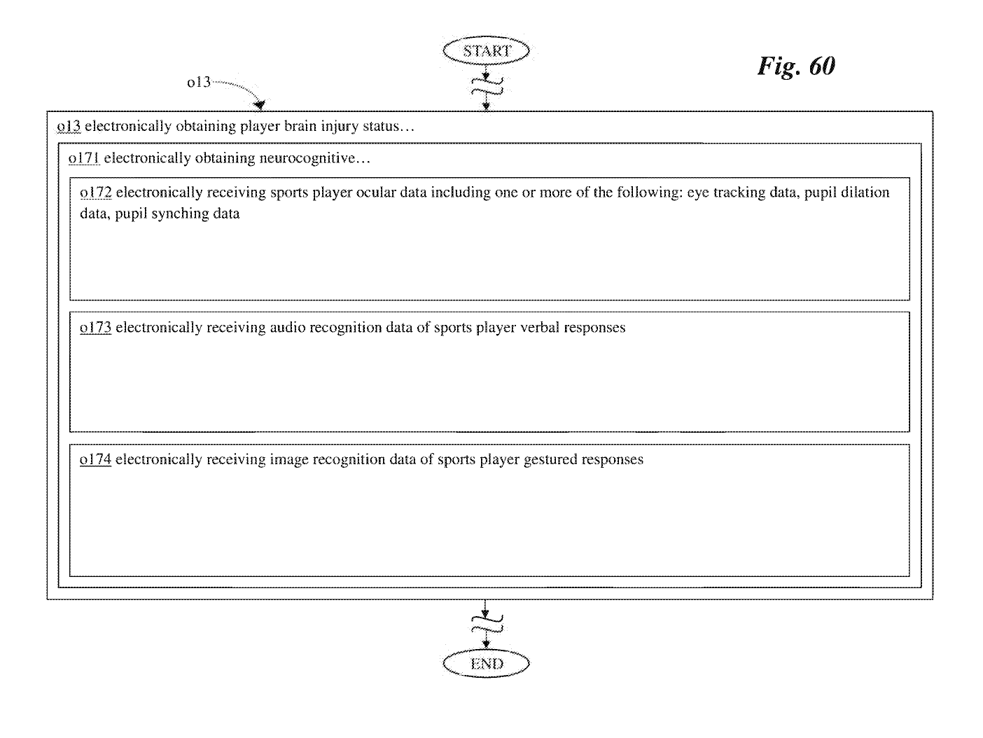

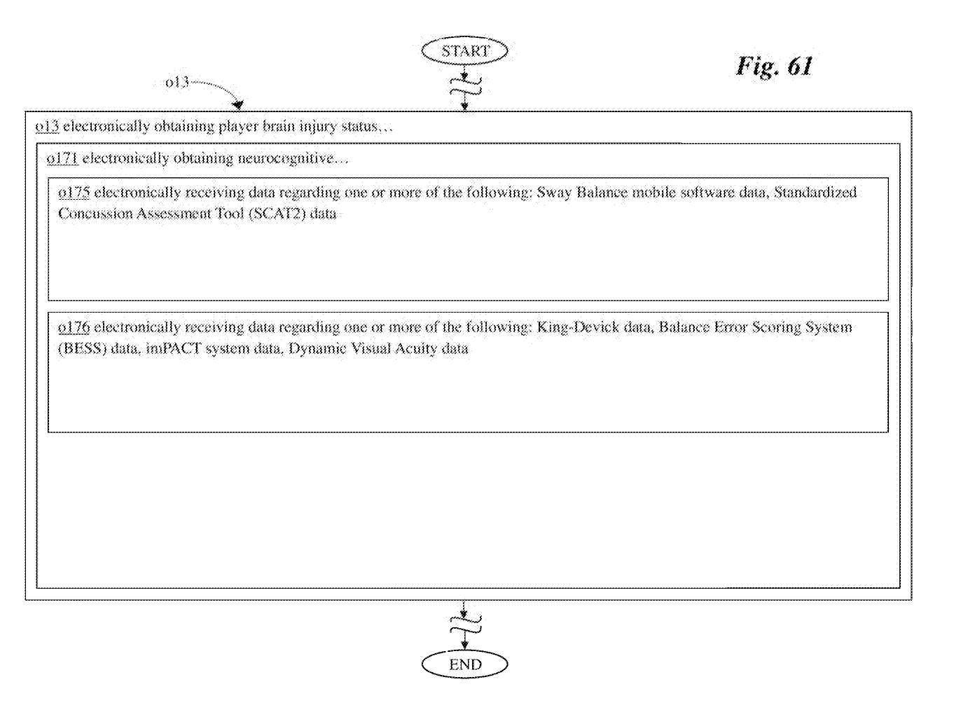

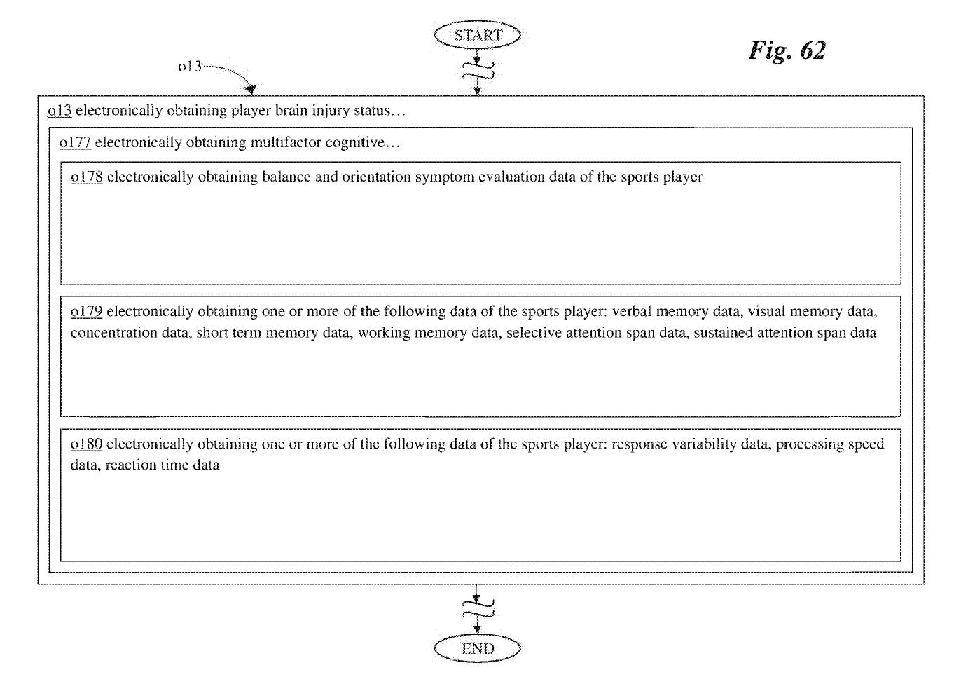

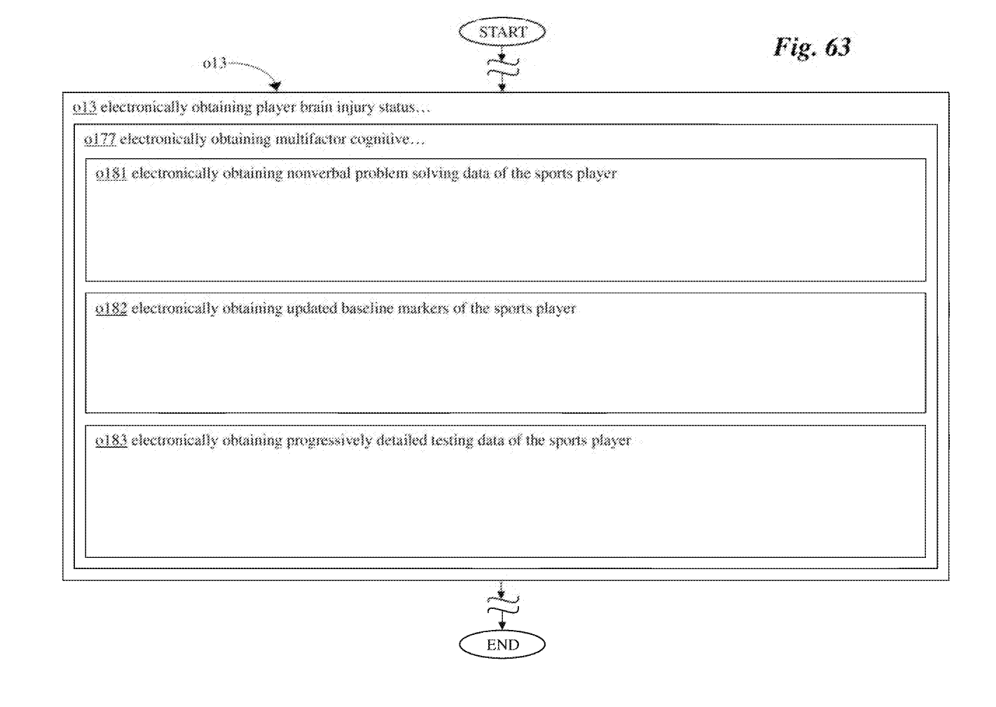

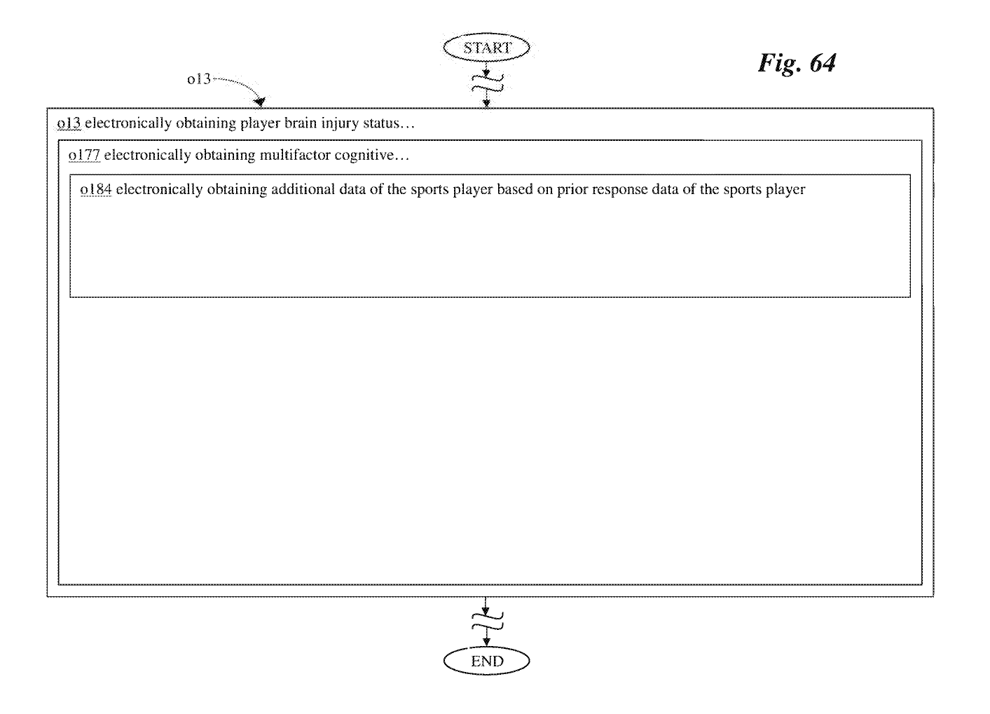

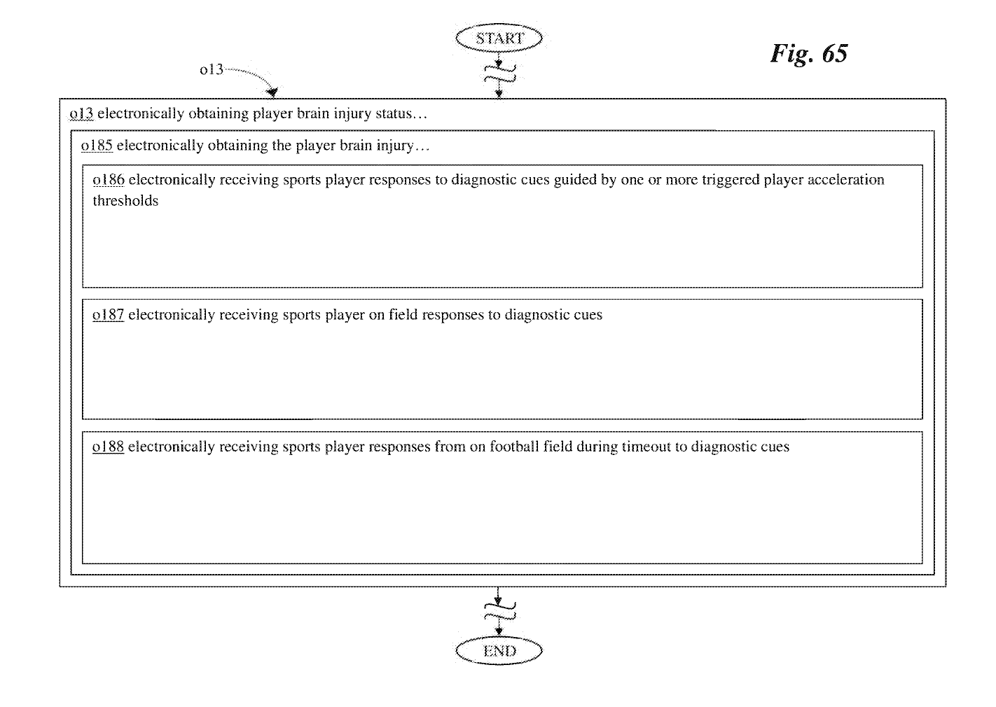

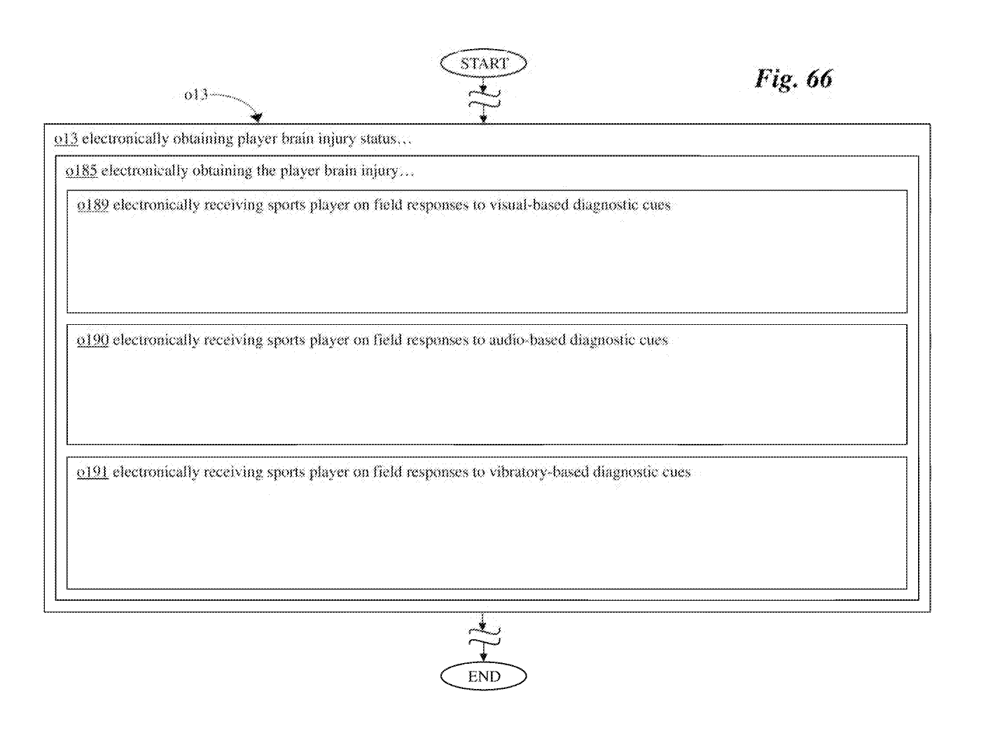

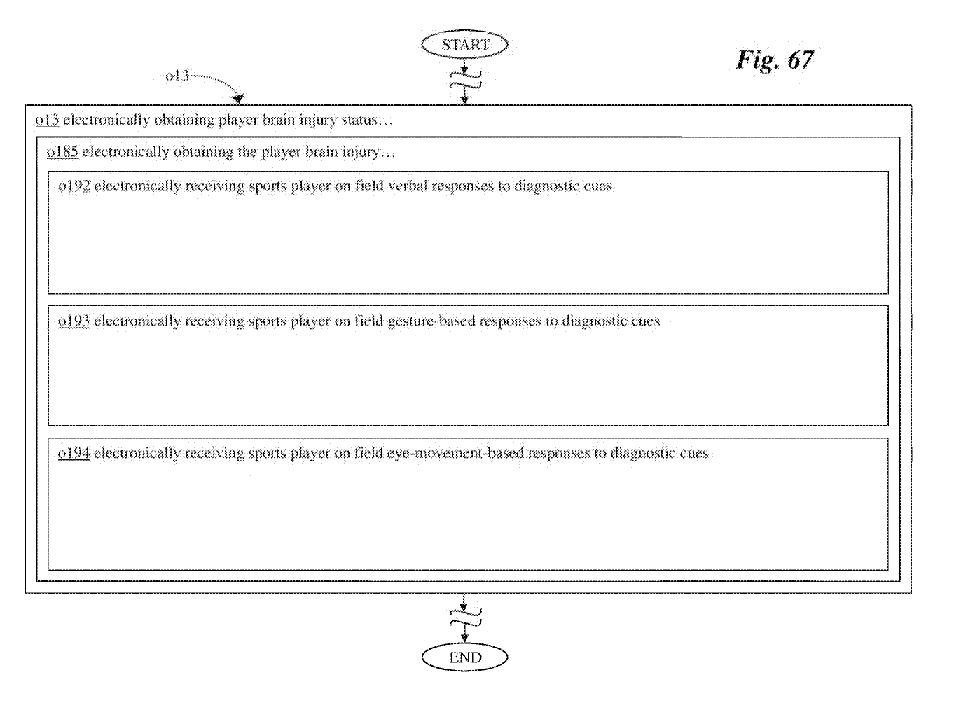

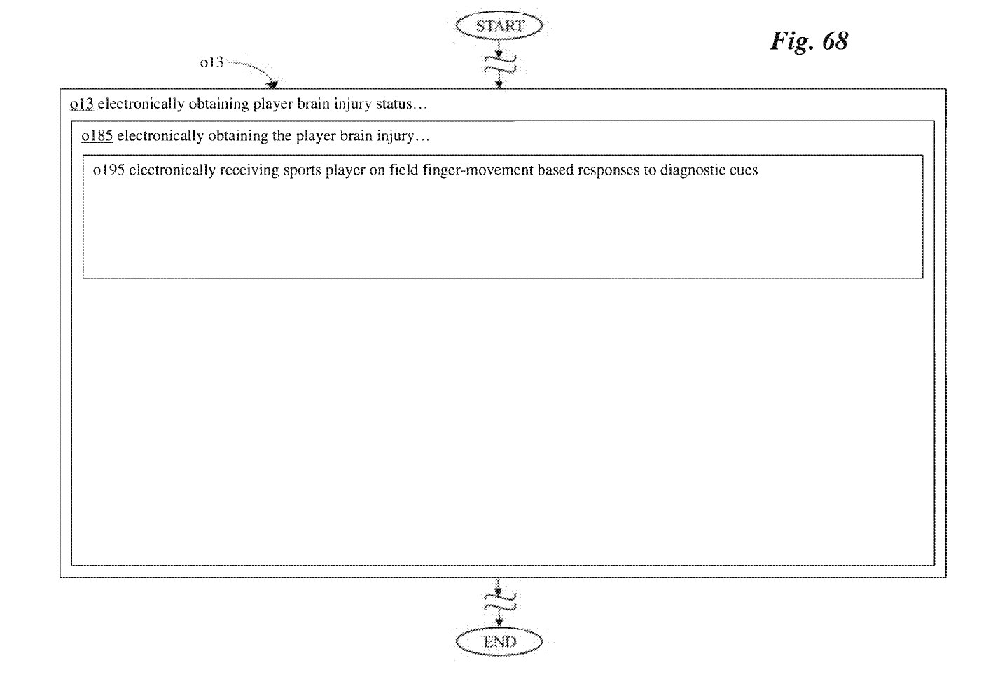

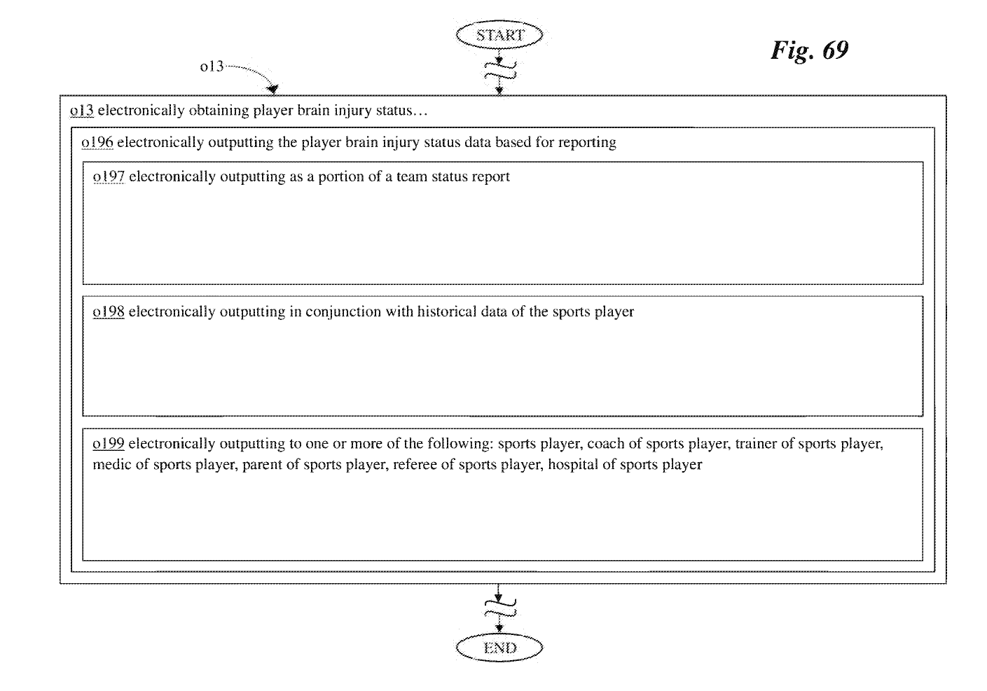

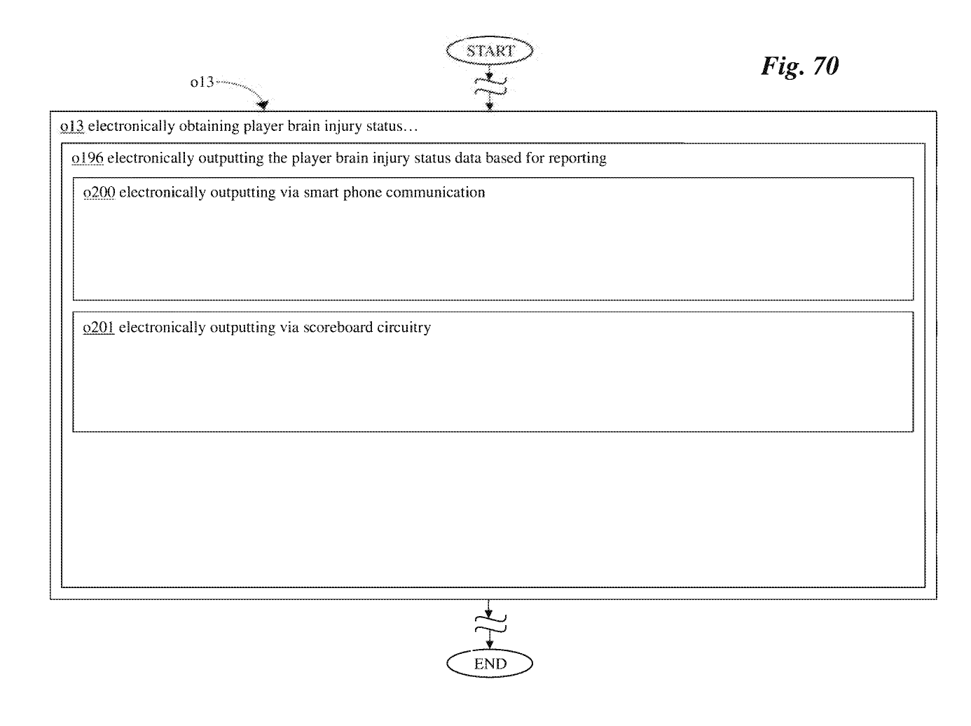

In accordance with 37 C.F.R. §1.84(h)(2), FIG. 1 shows “a view of a large machine or device in its entirety . . . broken into partial views . . . extended over several sheets” labeled FIG. 1-A through FIG. 1-F (Sheets 1-7 including FIG. 1). The “views on two or more sheets form, in effect, a single complete view, [and] the views on the several sheets . . . [are] so arranged that the complete figure can be assembled” from “partial views drawn on separate sheets . . . linked edge to edge. Thus, in FIG. 1, the partial view FIGS. 1-A through 1-F are ordered alphabetically, by increasing in columns from left to right, and increasing in rows top to bottom, as shown in the following table:

TABLE 1
Table showing alignment of enclosed drawings to form
partial schematic of one or more environments.

| Pos. (0, 0) | X-Position 1 | X-Position 2 | X-Position 3 |
|---|---|---|---|
| Y-Pos. 1 | (1, 1): FIG. | (1, 2): FIG. | (1, 3): FIG. |
| | 1-A | 1-B | 1-C |
| Y-Pos. 2 | (2, 1): FIG. | (2, 2): FIG. | (2, 3): FIG. |
| | 1-D | 1-E | 1-F |

In accordance with 37 C.F.R. §1.84(h)(2), FIG. 1 is “ . . . a view of a large machine or device in its entirety . . . broken into partial views . . . extended over several sheets . . . [with] no loss in facility of understanding the view.” The partial views drawn on the several sheets indicated in the above table are capable of being linked edge to edge, so that no partial view contains parts of another partial view. As here, “where views on two or more sheets form, in effect, a single complete view, the views on the several sheets are so arranged that the complete figure can be assembled without concealing any part of any of the views appearing on the various sheets.” 37 C.F.R. §1.84(h)(2).

It is noted that one or more of the partial views of the drawings may be blank, or may be absent of substantive elements (e.g., may show only lines, connectors, arrows, and/or the like). These drawings are included in order to assist readers of the application in assembling the single complete view from the partial sheet format required for submission by the USPTO, and, while their inclusion is not required and may be omitted in this or other applications without subtracting from the disclosed matter as a whole, their inclusion is proper, and should be considered and treated as intentional.

FIG. 1-A, when placed at position (1,1), forms at least a portion of a partially schematic diagram of an environment(s) and/or an implementation(s) of technologies described herein.

FIG. 1-B, when placed at position (1,2), forms at least a portion of a partially schematic diagram of an environment(s) and/or an implementation(s) of technologies described herein.

FIG. 1-C, when placed at position (1,3), forms at least a portion of a partially schematic diagram of an environment(s) and/or an implementation(s) of technologies described herein.

FIG. 1-D, when placed at position (2,1), forms at least a portion of a partially schematic diagram of an environment(s) and/or an implementation(s) of technologies described herein.

FIG. 1-E, when placed at position (2,2), forms at least a portion of a partially schematic diagram of an environment(s) and/or an implementation(s) of technologies described herein.

FIG. 1-F, when placed at position (2,3), forms at least a portion of a partially schematic diagram of an environment(s) and/or an implementation(s) of technologies described herein.

FIG. 2 shows a schematic diagram of implementation(s) of environment(s) and/or implementations(s) of one or more technologies described herein including food fabricator implementation(s) in communication with bio-info/data device implementation(s), with food supply implementation(s) and with big-data analytics implementation(s).

FIG. 3 shows a schematic diagram of implementation(s) of environment(s) and/or implementations(s) of one or more technologies described herein including fabricator communication system implementation(s).

FIG. 4 shows a schematic diagram of implementation(s) of environment(s) and/or implementations(s) of one or more technologies described herein including processing module implementation(s).

FIG. 5 through FIG. 8 show partially schematic diagrams of implementations of player acceleration data associated with a head modules.

FIG. 9 through FIG. 13 show partially schematic diagrams of implementation(s) of game time player status data modules.

FIG. 14 through FIG. 27 show partially schematic diagrams of an implementations of player brain injury status data modules.

FIG. 28 shows a high-level flowchart illustrating an operational flow o10 representing exemplary operations related to operation o11, operation o12, and operation o13.

FIGS. 29 and 38-44 show high-level flowcharts including exemplary implementations of operation o11 of FIG. 28.

FIGS. 30, 35, and 45-51 show high-level flowcharts including exemplary implementations of operation o12 of FIG. 28.

FIGS. 31-34, 36-37, and 52-70 show high-level flowcharts including exemplary implementations of operation o13 of FIG. 28.

### DETAILED DESCRIPTION

### Overview

In the following detailed description, reference is made to the accompanying drawings, which form a part hereof. In the drawings, similar symbols typically identify similar or identical components or items, unless context dictates otherwise. The illustrative embodiments described in the detailed description, drawings, and claims are not meant to be limiting. Other embodiments may be utilized, and other changes may be made, without departing from the spirit or scope of the subject matter presented here.

Thus, in accordance with various embodiments, computationally implemented methods, systems, circuitry, articles of manufacture, ordered chains of matter, and computer program products are designed to, among other things, provide an interface for that substantially as shown and described in the detailed description and/or drawings and/or elsewhere herein.

The present application uses formal outline headings for clarity of presentation. However, it is to be understood that the outline headings are for presentation purposes, and that different types of subject matter may be discussed throughout the application (e.g., device(s)/structure(s) may be described under process(es)/operations heading(s) and/or process(es)/operations may be discussed under structure(s)/process(es) headings; and/or descriptions of single topics may span two or more topic headings). Hence, the use of the formal outline headings is not intended to be in any way limiting.

The claims, description, and drawings of this application may describe one or more of the instant technologies in operational/functional language, for example as a set of operations to be performed by a computer. Such operational/functional description in most instances would be understood by one skilled the art as specifically-configured hardware (e.g., because a general purpose computer in effect becomes a special purpose computer once it is programmed to perform particular functions pursuant to instructions from program software (e.g., a high-level computer program serving as a hardware specification)).

The claims, description, and drawings of this application may describe one or more of the instant technologies in operational/functional language, for example as a set of operations to be performed by a computer. Such operational/functional description in most instances would be understood by one skilled the art as specifically-configured hardware (e.g., because a general purpose computer in effect becomes a special purpose computer once it is programmed to perform particular functions pursuant to instructions from program software).

### Operational/Functional Language is a Concrete Specification for Physical Implementation

Importantly, although the operational/functional descriptions described herein are understandable by the human mind, they are not abstract ideas of the operations/functions divorced from computational implementation of those operations/functions. Rather, the operations/functions represent a specification for the massively complex computational machines or other means. As discussed in detail below, the operational/functional language must be read in its proper technological context, i.e., as concrete specifications for physical implementations.

The logical operations/functions described herein are a distillation of machine specifications or other physical mechanisms specified by the operations/functions such that the otherwise inscrutable machine specifications may be comprehensible to the human mind. The distillation also allows one of skill in the art to adapt the operational/functional description of the technology across many different specific vendors' hardware configurations or platforms, without being limited to specific vendors' hardware configurations or platforms.

Some of the present technical description (e.g., detailed description, drawings, claims, etc.) may be set forth in terms of logical operations/functions. As described in more detail in the following paragraphs, these logical operations/functions are not representations of abstract ideas, but rather representative of static or sequenced specifications of various hardware elements. Differently stated, unless context dictates otherwise, the logical operations/functions will be understood by those of skill in the art to be representative of static or sequenced specifications of various hardware elements. This is true because tools available to one of skill in the art to implement technical disclosures set forth in operational/functional formats—tools in the form of a high-level programming language (e.g., C, java, visual basic), etc.), or tools in the form of Very high speed Hardware Description Language (“VHDL,” which is a language that uses text to describe logic circuits)—are generators of static or sequenced specifications of various hardware configurations. This fact is sometimes obscured by the broad term “software,” but, as shown by the following explanation, those skilled in the art understand that what is termed “software” is a shorthand for a massively complex interchaining/specification of ordered-matter elements. The term “ordered-matter elements” may refer to physical components of computation, such as assemblies of electronic logic gates, molecular computing logic constituents, quantum computing mechanisms, etc.

For example, a high-level programming language is a programming language with strong abstraction, e.g., multiple levels of abstraction, from the details of the sequential organizations, states, inputs, outputs, etc., of the machines that a high-level programming language actually specifies. In order to facilitate human comprehension, in many instances, high-level programming languages resemble or even share symbols with natural languages.

It has been argued that because high-level programming languages use strong abstraction (e.g., that they may resemble or share symbols with natural languages), they are therefore a “purely mental construct.” (e.g., that “software”—a computer program or computer programming—is somehow an ineffable mental construct, because at a high level of abstraction, it can be conceived and understood in the human mind). This argument has been used to characterize technical description in the form of functions/operations as somehow “abstract ideas.” In fact, in technological arts (e.g., the information and communication technologies) this is not true.

The fact that high-level programming languages use strong abstraction to facilitate human understanding should not be taken as an indication that what is expressed is an abstract idea. In fact, those skilled in the art understand that just the opposite is true. If a high-level programming language is the tool used to implement a technical disclosure in the form of functions/operations, those skilled in the art will recognize that, far from being abstract, imprecise, “fuzzy,” or “mental” in any significant semantic sense, such a tool is instead a near incomprehensibly precise sequential specification of specific computational machines—the parts of which are built up by activating/selecting such parts from typically more general computational machines over time (e.g., clocked time). This fact is sometimes obscured by the superficial similarities between high-level programming languages and natural languages. These superficial similarities also may cause a glossing over of the fact that high-level programming language implementations ultimately perform valuable work by creating/controlling many different computational machines.

The many different computational machines that a high-level programming language specifies are almost unimaginably complex. At base, the hardware used in the computational machines typically consists of some type of ordered matter (e.g., traditional electronic devices (e.g., transistors), deoxyribonucleic acid (DNA), quantum devices, mechanical switches, optics, fluidics, pneumatics, optical devices (e.g., optical interference devices), molecules, etc.) that are arranged to form logic gates. Logic gates are typically physical devices that may be electrically, mechanically, chemically, or otherwise driven to change physical state in order to create a physical reality of Boolean logic.

Logic gates may be arranged to form logic circuits, which are typically physical devices that may be electrically, mechanically, chemically, or otherwise driven to create a physical reality of certain logical functions. Types of logic circuits include such devices as multiplexers, registers, arithmetic logic units (ALUs), computer memory, etc., each type of which may be combined to form yet other types of physical devices, such as a central processing unit (CPU)—the best known of which is the microprocessor. A modern microprocessor will often contain more than one hundred million logic gates in its many logic circuits (and often more than a billion transistors).

The logic circuits forming the microprocessor are arranged to provide a microarchitecture that will carry out the instructions defined by that microprocessor's defined Instruction Set Architecture. The Instruction Set Architecture is the part of the microprocessor architecture related to programming, including the native data types, instructions, registers, addressing modes, memory architecture, interrupt and exception handling, and external Input/Output.

The Instruction Set Architecture includes a specification of the machine language that can be used by programmers to use/control the microprocessor. Since the machine language instructions are such that they may be executed directly by the microprocessor, typically they consist of strings of binary digits, or bits. For example, a typical machine language instruction might be many bits long (e.g., 32, 64, or 128 bit strings are currently common). A typical machine language instruction might take the form “11110000101011110000111100111111” (a 32 bit instruction).

It is significant here that, although the machine language instructions are written as sequences of binary digits, in actuality those binary digits specify physical reality. For example, if certain semiconductors are used to make the operations of Boolean logic a physical reality, the apparently mathematical bits “1” and “0” in a machine language instruction actually constitute shorthand that specifies the application of specific voltages to specific wires. For example, in some semiconductor technologies, the binary number “1” (e.g., logical “1”) in a machine language instruction specifies around +5 volts applied to a specific “wire” (e.g., metallic traces on a printed circuit board) and the binary number “0” (e.g., logical “0”) in a machine language instruction specifies around −5 volts applied to a specific “wire.” In addition to specifying voltages of the machines' configuration, such machine language instructions also select out and activate specific groupings of logic gates from the millions of logic gates of the more general machine. Thus, far from abstract mathematical expressions, machine language instruction programs, even though written as a string of zeros and ones, specify many, many constructed physical machines or physical machine states.

Machine language is typically incomprehensible by most humans (e.g., the above example was just ONE instruction, and some personal computers execute more than two billion instructions every second). Thus, programs written in machine language—which may be tens of millions of machine language instructions long—are incomprehensible. In view of this, early assembly languages were developed that used mnemonic codes to refer to machine language instructions, rather than using the machine language instructions' numeric values directly (e.g., for performing a multiplication operation, programmers coded the abbreviation “mult,” which represents the binary number “011000” in MIPS machine code). While assembly languages were initially a great aid to humans controlling the microprocessors to perform work, in time the complexity of the work that needed to be done by the humans outstripped the ability of humans to control the microprocessors using merely assembly languages.

At this point, it was noted that the same tasks needed to be done over and over, and the machine language necessary to do those repetitive tasks was the same. In view of this, compilers were created. A compiler is a device that takes a statement that is more comprehensible to a human than either machine or assembly language, such as “add 2+2 and output the result,” and translates that human understandable statement into a complicated, tedious, and immense machine language code (e.g., millions of 32, 64, or 128 bit length strings). Compilers thus translate high-level programming language into machine language.

This compiled machine language, as described above, is then used as the technical specification which sequentially constructs and causes the interoperation of many different computational machines such that humanly useful, tangible, and concrete work is done. For example, as indicated above, such machine language—the compiled version of the higher-level language—functions as a technical specification which selects out hardware logic gates, specifies voltage levels, voltage transition timings, etc., such that the humanly useful work is accomplished by the hardware.

Thus, a functional/operational technical description, when viewed by one of skill in the art, is far from an abstract idea. Rather, such a functional/operational technical description, when understood through the tools available in the art such as those just described, is instead understood to be a humanly understandable representation of a hardware specification, the complexity and specificity of which far exceeds the comprehension of most any one human. With this in mind, those skilled in the art will understand that any such operational/functional technical descriptions—in view of the disclosures herein and the knowledge of those skilled in the art—may be understood as operations made into physical reality by (a) one or more interchained physical machines, (b) interchained logic gates configured to create one or more physical machine(s) representative of sequential/combinatorial logic(s), (c) interchained ordered matter making up logic gates (e.g., interchained electronic devices (e.g., transistors), DNA, quantum devices, mechanical switches, optics, fluidics, pneumatics, molecules, etc.) that create physical reality representative of logic(s), or (d) virtually any combination of the foregoing. Indeed, any physical object which has a stable, measurable, and changeable state may be used to construct a machine based on the above technical description. Charles Babbage, for example, constructed the first computer out of wood and powered by cranking a handle.

Thus, far from being understood as an abstract idea, those skilled in the art will recognize a functional/operational technical description as a humanly-understandable representation of one or more almost unimaginably complex and time sequenced hardware instantiations. The fact that functional/operational technical descriptions might lend themselves readily to high-level computing languages (or high-level block diagrams for that matter) that share some words, structures, phrases, etc. with natural language simply cannot be taken as an indication that such functional/operational technical descriptions are abstract ideas, or mere expressions of abstract ideas. In fact, as outlined herein, in the technological arts this is simply not true. When viewed through the tools available to those of skill in the art, such functional/operational technical descriptions are seen as specifying hardware configurations of almost unimaginable complexity.

As outlined above, the reason for the use of functional/operational technical descriptions is at least twofold. First, the use of functional/operational technical descriptions allows near-infinitely complex machines and machine operations arising from interchained hardware elements to be described in a manner that the human mind can process (e.g., by mimicking natural language and logical narrative flow). Second, the use of functional/operational technical descriptions assists the person of skill in the art in understanding the described subject matter by providing a description that is more or less independent of any specific vendor's piece(s) of hardware.

The use of functional/operational technical descriptions assists the person of skill in the art in understanding the described subject matter since, as is evident from the above discussion, one could easily, although not quickly, transcribe the technical descriptions set forth in this document as trillions of ones and zeroes, billions of single lines of assembly-level machine code, millions of logic gates, thousands of gate arrays, or any number of intermediate levels of abstractions. However, if any such low-level technical descriptions were to replace the present technical description, a person of skill in the art could encounter undue difficulty in implementing the disclosure, because such a low-level technical description would likely add complexity without a corresponding benefit (e.g., by describing the subject matter utilizing the conventions of one or more vendor-specific pieces of hardware). Thus, the use of functional/operational technical descriptions assists those of skill in the art by separating the technical descriptions from the conventions of any vendor-specific piece of hardware.

In view of the foregoing, the logical operations/functions set forth in the present technical description are representative of static or sequenced specifications of various ordered-matter elements, in order that such specifications may be comprehensible to the human mind and adaptable to create many various hardware configurations. The logical operations/functions disclosed herein should be treated as such, and should not be disparagingly characterized as abstract ideas merely because the specifications they represent are presented in a manner that one of skill in the art can readily understand and apply in a manner independent of a specific vendor's hardware implementation.

Those having skill in the art will recognize that the state of the art has progressed to the point where there is little distinction left between hardware, software (e.g., a high-level computer program serving as a hardware specification), and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software (e.g., a high-level computer program serving as a hardware specification), and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software (e.g., a high-level computer program serving as a hardware specification) implementation; or, yet again alternatively, the implementer may opt for some combination of hardware, software (e.g., a high-level computer program serving as a hardware specification), and/or firmware in one or more machines, compositions of matter, and articles of manufacture, limited to patentable subject matter under 35 USC 101. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software (e.g., a high-level computer program serving as a hardware specification), and or firmware.

In some implementations described herein, logic and similar implementations may include computer programs or other control structures. Electronic circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software (e.g., a high-level computer program serving as a hardware specification) or firmware, or of gate arrays or programmable hardware, such as by performing a reception of or a transmission of one or more instructions in relation to one or more operations described herein. Alternatively or additionally, in some variants, an implementation may include special-purpose hardware, software (e.g., a high-level computer program serving as a hardware specification), firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or invoking circuitry for enabling, triggering, coordinating, requesting, or otherwise causing one or more occurrences of virtually any functional operation described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled//implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

The term module, as used in the foregoing/following disclosure, may refer to a collection of one or more components that are arranged in a particular manner, or a collection of one or more general-purpose components that may be configured to operate in a particular manner at one or more particular points in time, and/or also configured to operate in one or more further manners at one or more further times. For example, the same hardware, or same portions of hardware, may be configured/reconfigured in sequential/parallel time(s) as a first type of module (e.g., at a first time), as a second type of module (e.g., at a second time, which may in some instances coincide with, overlap, or follow a first time), and/or as a third type of module (e.g., at a third time which may, in some instances, coincide with, overlap, or follow a first time and/or a second time), etc. Reconfigurable and/or controllable components (e.g., general purpose processors, digital signal processors, field programmable gate arrays, etc.) are capable of being configured as a first module that has a first purpose, then a second module that has a second purpose and then, a third module that has a third purpose, and so on. The transition of a reconfigurable and/or controllable component may occur in as little as a few nanoseconds, or may occur over a period of minutes, hours, or days.

In some such examples, at the time the component is configured to carry out the second purpose, the component may no longer be capable of carrying out that first purpose until it is reconfigured. A component may switch between configurations as different modules in as little as a few nanoseconds. A component may reconfigure on-the-fly, e.g., the reconfiguration of a component from a first module into a second module may occur just as the second module is needed. A component may reconfigure in stages, e.g., portions of a first module that are no longer needed may reconfigure into the second module even before the first module has finished its operation. Such reconfigurations may occur automatically, or may occur through prompting by an external source, whether that source is another component, an instruction, a signal, a condition, an external stimulus, or similar.

For example, a central processing unit of a personal computer may, at various times, operate as a module for displaying graphics on a screen, a module for writing data to a storage medium, a module for receiving user input, and a module for multiplying two large prime numbers, by configuring its logical gates in accordance with its instructions. Such reconfiguration may be invisible to the naked eye, and in some embodiments may include activation, deactivation, and/or re-routing of various portions of the component, e.g., switches, logic gates, inputs, and/or outputs. Thus, in the examples found in the foregoing/following disclosure, if an example includes or recites multiple modules, the example includes the possibility that the same hardware may implement more than one of the recited modules, either contemporaneously or at discrete times or timings. The implementation of multiple modules, whether using more components, fewer components, or the same number of components as the number of modules, is merely an implementation choice and does not generally affect the operation of the modules themselves. Accordingly, it should be understood that any recitation of multiple discrete modules in this disclosure includes implementations of those modules as any number of underlying components, including, but not limited to, a single component that reconfigures itself over time to carry out the functions of multiple modules, and/or multiple components that similarly reconfigure, and/or special purpose reconfigurable components.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextel, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof, limited to patentable subject matter under 35 U.S.C. 101; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs (e.g., graphene based circuitry). Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will recognize that at least a portion of the devices and/or processes described herein can be integrated into an image processing system. Those having skill in the art will recognize that a typical image processing system generally includes one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system may be implemented utilizing suitable commercially available components, such as those typically found in digital still systems and/or digital motion systems.

Those skilled in the art will recognize that at least a portion of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

Those skilled in the art will recognize that at least a portion of the devices and/or processes described herein can be integrated into a mote system. Those having skill in the art will recognize that a typical mote system generally includes one or more memories such as volatile or non-volatile memories, processors such as microprocessors or digital signal processors, computational entities such as operating systems, user interfaces, drivers, sensors, actuators, applications programs, one or more interaction devices (e.g., an antenna USB ports, acoustic ports, etc.), control systems including feedback loops and control motors (e.g., feedback for sensing or estimating position and/or velocity; control motors for moving and/or adjusting components and/or quantities). A mote system may be implemented utilizing suitable components, such as those found in mote computing/communication systems. Specific examples of such components entail such as Intel Corporation's and/or Crossbow Corporation's mote components and supporting hardware, software, and/or firmware.

For the purposes of this application, “cloud” computing may be understood as described in the cloud computing literature. For example, cloud computing may be methods and/or systems for the delivery of computational capacity and/or storage capacity as a service. The “cloud” may refer to one or more hardware and/or software components that deliver or assist in the delivery of computational and/or storage capacity, including, but not limited to, one or more of a client, an application, a platform, an infrastructure, and/or a server The cloud may refer to any of the hardware and/or software associated with a client, an application, a platform, an infrastructure, and/or a server. For example, cloud and cloud computing may refer to one or more of a computer, a processor, a storage medium, a router, a switch, a modem, a virtual machine (e.g., a virtual server), a data center, an operating system, a middleware, a firmware, a hardware back-end, a software back-end, and/or a software application. A cloud may refer to a private cloud, a public cloud, a hybrid cloud, and/or a community cloud. A cloud may be a shared pool of configurable computing resources, which may be public, private, semi-private, distributable, scaleable, flexible, temporary, virtual, and/or physical. A cloud or cloud service may be delivered over one or more types of network, e.g., a mobile communication network, and the Internet.

As used in this application, a cloud or a cloud service may include one or more of infrastructure-as-a-service (“IaaS”), platform-as-a-service (“PaaS”), software-as-a-service (“SaaS”), and/or desktop-as-a-service (“DaaS”). As a non-exclusive example, IaaS may include, e.g., one or more virtual server instantiations that may start, stop, access, and/or configure virtual servers and/or storage centers (e.g., providing one or more processors, storage space, and/or network resources on-demand, e.g., EMC and Rackspace). PaaS may include, e.g., one or more software and/or development tools hosted on an infrastructure (e.g., a computing platform and/or a solution stack from which the client can create software interfaces and applications, e.g., Microsoft Azure). SaaS may include, e.g., software hosted by a service provider and accessible over a network (e.g., the software for the application and/or the data associated with that software application may be kept on the network, e.g., Google Apps, SalesForce). DaaS may include, e.g., providing desktop, applications, data, and/or services for the user over a network (e.g., providing a multi-application framework, the applications in the framework, the data associated with the applications, and/or services related to the applications and/or the data over the network, e.g., Citrix). The foregoing is intended to be exemplary of the types of systems and/or methods referred to in this application as “cloud” or “cloud computing” and should not be considered complete or exhaustive.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

To the extent that formal outline headings are present in this application, it is to be understood that the outline headings are for presentation purposes, and that different types of subject matter may be discussed throughout the application (e.g., device(s)/structure(s) may be described under process(es)/operations heading(s) and/or process(es)/operations may be discussed under structure(s)/process(es) headings; and/or descriptions of single topics may span two or more topic headings). Hence, any use of formal outline headings in this application is for presentation purposes, and is not intended to be in any way limiting.

Throughout this application, examples and lists are given, with parentheses, the abbreviation “e.g.,” or both. Unless explicitly otherwise stated, these examples and lists are merely exemplary and are non-exhaustive. In most cases, it would be prohibitive to list every example and every combination. Thus, smaller, illustrative lists and examples are used, with focus on imparting understanding of the claim terms rather than limiting the scope of such terms.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Although one or more users maybe shown and/or described herein, e.g., in FIG. 1, and other places, as a single illustrated figure, those skilled in the art will appreciate that one or more users may be representative of one or more human users, robotic users (e.g., computational entity), and/or substantially any combination thereof (e.g., a user may be assisted by one or more robotic agents) unless context dictates otherwise. Those skilled in the art will appreciate that, in general, the same may be said of “sender” and/or other entity-oriented terms as such terms are used herein unless context dictates otherwise.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

### High-Level System Architecture

FIG. 1, showing how FIGS. 1-A-1-F are assembled to form a complete view of an entire system, of which at least a portion will be described in more detail. An overview of the entire system of FIG. 1 is now described herein.

### Impact Sensor System 12

Referring now to FIG. 1-A, one or more machines/articles/compositions/processes related to traumatic brain injuries (e.g. traumatic brain injuries (TBI), mild traumatic brain injuries (mTBI), concussions, etc.) are depicted as including impact sensor system 12 to detect, sense, measure, or otherwise determine forces related to impact due to collision of a sports player with one or more other sports players, ground, equipment, game balls or other game devices, objects, etc. in which force is imparted to a specified location of a sports player (e.g. on or near player's head). Although not limiting in nature, a particular sort of player impact that can be of concern is that which may have potential for injury to a player's brain such as a traumatic brain injury. Determination of forces imparted to a player's head can be done through detection of positive or negative linear or angular velocity, acceleration, jerk, etc. of a player in general, and a player's head (e.g. player's head 12e) in particular. Although this detection may not be a final indicator that a traumatic brain injury has occurred, it can be used along with other factors to flag to a certain degree of accuracy the possibility that a brain injury has occurred with a player.

Such detection of positive or negative linear or angular velocity, acceleration (e.g. six-axis accelerometers), jerk, etc. of a player's head can include impact sensor systems such as having head sensors integrated into sports helmets (e.g. football helmet 12f with face shield 12g for football player 12m, such as for example football helmets with the Riddell Insite Response System (only requires football helmet, does not use or require a face shield), baseball helmet 12k for baseball player 12l, BrainSentry peel-n-stick sensor system that affixes to outer surfaces of helmets such as football helmets and includes a display to alert of status. Other helmets can have impact sensors as well such as hard or soft helmets for other sports such as lacrosse, hockey, bicycling, wrestling, soccer, etc.). Other implementations of impact sensor system 12 can include impact sensors integrated with banded devices (e.g. banded display 12h or wristband 12i), head-bands (e.g. head-band 12j), or glasses (e.g. sports glasses 12n), or skullcaps or beanies such as for soccer (e.g. skullcap or beanie 12d, such as skullcap or beanie Checklight MC-10 by Rebok, which energizes one or more indicator lights on the beanie if an impact threshold has been exceeded). Impact sensors can also be integrated into patch (generally affixed to skin) or button (generally small sensor package) systems for helmeted or non-helmeted sports such as basketball, bicycling, soccer, baseball, etc. (e.g. button or patch sensor 12a worn behind ear 12b, such as X-patch system by X2 Biosystems or conventional button sensors). Other impact sensors can be located in mouth guards such as having a 6-axis accelerometer inside of X-Guard mouth guard by X2 Bioystems. Other impact sensor systems can use RFID-based (radio frequency identification based) sensors with RFID emitters (e.g. RFID emitter 12c) affixed to the player and RFID sensors positioned on the playing field to obtain player linear or angular position, velocity, acceleration, jerk data (e.g. conventional RFID technology provided by Zebra Technologies, Inc.).

Various signaling devices such as lights, displays, or audio emitters can be integrated with the impact sensor system 12 such as into or on helmets, head-bands, wrist-bands, to apprise both sports players and others (such as coach, referee, or trainer) of sports player impact status, such as alerting when impact has been over various predetermined thresholds. The impact sensor system 12 also includes communication and configuration capabilities as further described below. These communication and configuration capabilities allow for transmission of impact data detected by the impact sensor system 12 to other systems described herein and to also receive status and configuration information from these other systems. Through this inter-communication and configuration between various other systems and the impact sensor system 12, not only sports player impact data is detected as with conventional approaches but in addition provision can be made for player testing, status, and access management to be integrated with player impact sensing for updating status and systems configurations therebetween.

### Player Testing System 14

Referring now to FIG. 1, e.g. FIGS. 1-B and 1-E, one or more machines/articles/compositions/processes related to traumatic brain injuries (e.g. TBI, mTBI, concussion, etc.) are depicted as including player testing system 14 to interrogate, analyze, monitor, or otherwise assess, etc. at least to an initial degree cognitive, neurological, (otherwise known as neurocognitive), or other brain-related performance levels of a sports player through neurocognitive testing interaction with the sports player or through physiological monitoring of the sports player. Such brain-related performance assessment can be in particular associated with possible occurrence of concussion or other traumatic brain injury of the sports player due to impact imparted to the sports player. Estimates have included somewhere between 2 to 4 million sports related concussions occurring in the United States per year with an estimated 65% to 85% of these concussions being left unreported at least in the initial few days after a concussion has occurred. The gravity of these estimates is better understood when viewed in the context of other estimates having to do with a condition that affects primarily teens since their brains are still developing a great deal relative to adults in general. Second impact syndrome occurs when a second concussion is experienced within approximately 7 days after a first concussion has occurred. Estimates include an approximate 50% possibility of a second concussion under a second impact syndrome scenario causing death and another approximate 50% possibility of a second impact under a second impact syndrome scenario causing severe brain injury. Integration of the player testing system 14 with the other systems discussed herein seeks to in part address high levels of unreported, undiagnosed cases of sports-related traumatic brain injuries. As described further below, the player testing system 14 is integrated with the impact sensing system 12 and other systems described herein so that coordination between the systems is handled in an automated or semi-automated way such that onfield administration of brain-related performance testing and other responsive measures can be less burdensome than otherwise available. The player testing system 14 can include the following devices to implement testing systems and processes described below through integrated image displays, audio emitters, cameras, audio microphones, tactical input (e.g. touch pad, gesture recognition, etc.): medical instrumentation 14a (e.g. image, infrared, fMRI, CAT, or other scanning devices, etc.) wrist or other banded devices 14b, helmet (e.g. football) 14c with shield/visor 14d, mobile device 14e, computer monitor 14f, wrist display 14g, smart phone 14h, beanie 14i, sports glasses 14j, band (e.g. headband) 14k, behind ear 14l device 14m, sports cap 14n, and implant 14o.

Aspects of neurocognitive testing of a sports player by the player testing system 14 can include numerous types of automated or semi-automated testing such as through assessment of sports player eye-tracking, pupil dilation, pupil alignment, pupil synching, etc. as determined through image recognition or other tracking devices. Examples of conventional eye tracking testing include mild traumatic brain injury (mTBI) testing are discussed at https://www.braintrauma.org/research-at-btf/concussion-diagnostics/, http://www.brainline.org/content/multimedia.php?id=6250, and http://www.forbes.com/sites/robertglatter/2014/12/17/%20new-eye-tracking-technology-promising-as-biomarker-for-brain-injury-and-function/including recent research indicating that shearing of connections in brain frontal area can cause attention and memory deficits in individuals that have suffered a mTBI. Deficits in attention have been correlated with abnormalities in smooth pursuit eye-movement in individuals with damage to these frontal connections. Since smooth pursuit eye-movement is the ability to track an object that is following a consistent and predictable path, tracking an individual's eye movements can be used to assess whether someone has attention deficits from a head injury shearing frontal brain connections. These conventional eye tracking assessments, which are dynamic tests of attention, such as staying focused on a moving object displayed on a screen, can take a little time as 30 seconds (hundreds of data points a second) versus 20 or 30 minutes for reaction tests that have static interaction, such as “hit the button when you see a yellow triangle” (having a few data points per second at best). The conventional eye tracking tests also do not depend on the motivation level of the participant so test-retest reliability is high since either the participant is tracking or is not tracking compared with other response tests that depend upon more overt participation. Other neurocognitive tests are still useful though to give other perspectives on a sports player's status so eye tracking should not be viewed as the only test available that is any good.

Another type of conventional eye-tracking technology that can be incorporated into the player testing system 14 is associated with Oculogica company, which has studied versions of its technology involving participants watching music videos or television content for about four minutes while the ratio of horizontal to vertical eye movements is measured. In neurologically normal participants, ratios are nearly to 1:1, with horizontal movements essentially equaling vertical movements. With nerve damage or brain swelling pressing nerves, abnormal eye movement ratios can reflect the affected nerve such as occurring with traumatic brain injury (TBI) or mild traumatic brain injury (mTBI also known as concussion). This approach by Oculogica can provide potential for classifying and quantitating extent of brain injury.

Conventional eye tracking glasses technology that can be incorporated into the player testing system 14 can be such as from exemplary companies as Applied Science Laboratories that offers the Mobile Eye-XP Eye Tracking Glasses at http://www.asleyetracking.com/Site/Products/MobileEyeXGGlasses/tabid/70/Default.asp x. Eye tracking can be implemented by the Player Testing System 14 through use of helmet shields (e.g. helmet shield 14d of football helmet 14c) or through sports glasses (e.g. sports glasses 14j) by image projection of an object for the sports player to track with their eyes such as on a portion of the inner surface of a helmet shield or sports glasses lens and also including a miniature camera incorporated in a sports helmet or sports glasses to track eye movement of the sports player. Other implementations for image display and camera capture of eye-movement for eye tracking can use portable devices (e.g. portable devices 14e or 14h), computer monitors (e.g. monitor 14f), or sports bands (e.g. sports band 14k for head, arm, etc. optionally having an e-paper display).

Other sports player testing by the player testing system 14 can include audio recognition of player verbal response feedback, tactile player feedback (e.g. joystick controlled feedback from player), etc. Further non-limiting examples of player testing aspects can include automated or semi-automated implementation of neurocognitive or other brain-related performance testing such as for exemplary purposes, similarly found in one or more portions from conventional testing protocols including, but not limited to, Sway Balance™ iOS mobile software by Sway Medical, LLC, Standardized Assessment of Concussion (SAC), Standardized Concussion Assessment Tool (SCAT2), King-Devick testing system, Balance Error Scoring System (BESS), imPACT™ (Immediate Post-Concussion Assessment and Cognitive Testing) system, Reaction Time including mechanical based testing, Dynamic Visual Acuity Testing, etc. Portions of other conventional neurocognitive protocols that can be used by the player testing system 14 can be in the form of conventional pencil-and-paper test (e.g. similar to SAC or SCAT2) that has been adapted for computer-automated input or tests that have already been computerized such as imPACT testing, as described at https://www.impacttest.com/products/? The-ImPACT-Test-2 as including demographic information, concussion history, learning disabilities, current concussion symptoms, and neurocognitive testing. The imPACT testing further includes neurocognitive testing having word discrimination (attention and verbal recognition memory), design memory (attention and visual recognition memory), “X's and O's” (visual working memory and processing speed), symbol matching (visual processing speed, learning and memory), color match (choice reaction time, impulse control and response inhibition), three letter memory (working memory and visual-motor response speed). Verbal memory, visual memory, processing speed, reaction time and symptom scores are used to determine when a concussion has occurred.

The player testing system 14 can use other forms of neurocognitive testing similar to such conventional neurocognitive testing systems as the automated neuropsychological assessment metrics (ANAM) by Vista Life Science, which according thereto “provides randomized stimuli on tasks that are well-established cognitive measures, and records accuracy and timing of response with millisecond sensitivity and has a special timing mechanism to ensure test/re-test reliability.” as stated at http://www.vistalifesciences.com/index.php/anam-intro.html.

The player testing system 14 can use other forms of neurocognitive testing similar to such conventional neurocognitive testing systems as that of HeadMinder by HeadMinder, Inc., which according thereto “consists of a set of computerized subtests that require simple patient responses on a standard keyboard and measure aspects of cognition typically associated with a brain dysfunction, such as reaction time, concentration and working memory, information processing speed and accuracy, and short-term and long-term memory. Tests may also include questionnaires tailored for each presenting problem. . . . HeadMinder scientists have implemented the only existing commercial system that uses advanced statistical models for measuring and monitoring change in cognitive functions. Each individual's initial test results are used as a baseline for comparison to future tests. The baseline allows the system to create a unique longitudinal profile: the individual is compared to himself or herself over time, thereby increasing the accuracy of the test. (Most traditional assessment measures compare individuals to a group average.) . . . Specialized statistics control for practice effects and reduce other sources of error. Our tests employ multiple alternate forms, and our server keeps track of which forms have already been administered.”

The player testing system 14 can use other forms of neurocognitive testing similar to such conventional neurocognitive testing systems as that of computerized cognitive testing by CogState Research http://cogstate.com/academic-2/measurement-of-cognition/#.VNrxMi62Jm4, which includes Visual Motor Function (Chase Test), Executive Function/Spatial Problem Solving (Groton Maze Learning Test and Set-Shifting Task), Psychomotor Function/Speed of Processing (Detection Task), Visual Attention/Vigilance (Identification Task), Visual Learning & Memory (One Card Learning Task, Continuous Paired Associated Learning Task, Groton Maze Learning Test-Delayed Recall), Verbal Learning & Memory (International Shopping List Task and International Shopping List Task: Delayed Recall), Attention/Working Memory (One Back Task and Two Back Task) Social Cognition (Social-Emotional Cognition Task).

These and other sports player testing protocols can be implemented through automated or semi-automated devices as part of sports player testing by the player testing system 14 to arrive at initial brain-related performance assessments. Hybrid combinations of these or other assessments can also be used as implementations of existing and other sports player testing protocols. In addition, electronic image recognition of electronic image data or electronic audio recognition of electronic audio data of sports player behavior, expression, or other sorts of output by the sports player can be performed. Electronic image or audio data related to sports player behavior, expression, or other output can be captured for electronic image recognition or electronic audio recognition in various locations. Examples of such locations for such sports player behavior can be either on or off the field of play (e.g. field, court, rink, mat, etc.) during play of a game, during game intermission or other break in play of game, during down-time of a sports player away from the game area (e.g. away from sports field, court, rink, etc.) on the sidelines or elsewhere off of the field, or during play of game by other sports players, during implementation of testing protocols with a sports player or at other times can also be used to complement or otherwise furnish testing input to assess neurocognitive or other brain-related performance status for the sports player.

The player testing system 14 can also use sports player implanted devices or other physiological blood testing devices to monitor indicators of TBI, mTBI, concussion, etc. For instance, use of serum biomarkers such as S-100B, GFAP, neuron specific enolase (NSE), etc. could potentially be used in part to evaluate sports players after a potentially brain-injurious impact. For instance, elevated levels of S-100B may have some use in predicting severity of a brain injury later after injury as discussed at http://www.forbes.com/sites/robertglatter/2013/11/02/seattle-based-company-x2-biosystems-poised-to-change-approach-to-evaluation-of-traumatic-brain-injury-and-concussions/3/. Other blood testing by the player testing system 14 could include testing for elevated levels in the blood of the brain-enriched protein calpain-cleaved αII-spectrin N-terminal fragment, known as SNTF, to predict severity of symptoms due to a brain-injurious impact to a sports player as discussed at http://www.uphs.upenn.edu/news/News_Releases/2014/11/concussion/.

The player testing system 14 also includes communication and configuration capabilities as further described below. These communication and configuration capabilities allow for transmission of data related to player testing as determined by the player testing system 14 to other systems described herein and to also receive status and configuration information from these other systems. Through this inter-communication and configuration between various other systems and the player testing system 14, not only player testing data is determined as with conventional approaches but in addition provision can be made for player impact sensing, status, and access management to be integrated with player testing for updating status and systems configurations therebetween.

### Player Status System 16

Referring now to FIG. 1-D, one or more machines/articles/compositions/processes related to traumatic brain injuries (e.g. traumatic brain injuries (TBI), mild traumatic brain injuries (mTBI), concussions, etc.) are depicted as including impact sensor system 12 to report, broadcast, signal, alert, update, inform, or otherwise notify users and those otherwise associated regarding status of one or more players being monitored, etc. by impact sensor system 12, player testing system 14, field access system 18, or player location system 20. Past, present, or predicted metrics inputted or derived from these various systems and other ad hoc inputs or systems sent to the player status system 16 are then presented to individuals and groups accordingly, such as including output of status for one or more players, fans, officials, reporters, spectators, parents, referees, etc. Outputs and participants of the player status system 16 can include, but are not limited to scoreboard output 16a, baseball participant 16b, football participant 16c, coaching staff participant 16d, medical trainer participant 16e, referee participant 16f, monitor output 16g, instrument output 16h, helmet output 16i, wrist output 16j, watch output 16k, band output 16l, cap output 16m, handheld output 16n, auditory output 16o, button-sized output 16p, hat output 16q, glasses output 16r, signal output 16s, speaker output 16t, emitter output 16u, and visor output 16v.

Status of the player status system 16 can include player position, impact experienced by player, player TBI testing status, data-based information, current and historical player field-position information, current and historical impact information, current and historical TBI testing information, etc. The player status system 16 can include query capabilities to retrieve additional position, impact, testing and other information. The player status system 16 can also include a user-interface that can be customized according to user status such as user being player, coach, trainer, medic, parent, referee, scoreboard, medic, hospital, etc. User interfaces can be integrated into hand-held, laptop, mobile, stand-based, or table-based devices. Communication involved with the player status system 16 can include such as when system transmits/receives information/data to/from impact sensor system, system transmits/receives information/data to/from player testing system, and system transmits/receives information/data to/from field access system.

### Field Access System 18

Referring now to FIG. 1-C, one or more machines/articles/compositions/processes related to traumatic brain injuries (e.g. traumatic brain injuries (TBI), mild traumatic brain injuries (mTBI), concussions, etc.) are depicted as including field access system 18 to receive player information regarding status of one or more players being monitored, etc. by impact sensor system 12, player testing system 14, player status system 16, or player location system 20. Past, present, or predicted metrics inputted or derived from these various systems and other ad hoc inputs or systems sent to the field access system 18 are then used to determine access parameters to use to control access of one or more players, officials, other personnel, or persons via the field access system 18. Controls are used to either physically bar or informationally bar recipients of the controls from entering into restricted areas such as an area of a playing field or an entire playing field or an entire stadium complex, etc. Controls using information to bar access can output information related to a player access control either visually, auditory means, tactical means, or other means, which can be integrated into clothing, gear, etc. either affixed to player(s) or separate from player(s). Control aspects of the field access system 18 can include, but are not limited to computer control 18a, stadium control 18b, signal control 18c, speaker control 18d, emitter control 18e, gate control 18f, auditory control 18g, button control 18h, hat control 18i, helmet control 18j, visor control 18k, wrist control 18l, watch control 18m, band control 18n, cap control 18o, or glasses control 18p.

The field access system 18 can include controlling player access to: football, soccer, lacrosse, baseball, fields, etc., basketball, squash, racquetball courts, etc., wrestling mats, hockey rinks, etc. The field access system can include physical, electromagnetic, audio, or visual based player barriers in which physical barriers can include gated access to field, electromagnetic can include irritant-based output, audio can include directed interfering sound to player, visual can include blocked, altered, irritated player vision such as with helmet shield, glasses, goggles can include light-based or audio-based, field-mounted signaling to coach, referee, trainer, players, fans, etc., wearable signaling such as by arm, wrist, head, trunk, etc. and mounted signaling. As show the field access system 18 transmits/receives information/data to/from impact sensor system, transmits/receives information/data to/from player status system, or transmits/receives information/data to/from field access system.

### Player Location System 20

Referring now to FIG. 1-F, one or more machines/articles/compositions/processes related to traumatic brain injuries (e.g. traumatic brain injuries (TBI), mild traumatic brain injuries (mTBI), concussions, etc.) are depicted as including player location system 20 to receive player information regarding status of one or more players being monitored, etc. by impact sensor system 12, player testing system 14, player status system 16, or field access system 18. Past, present or predicted metrics inputted or derived from these various systems and other ad hoc inputs or systems sent to the player location system 20 are then used to assess various requirements for player location information determined by the player location system 20. Player location is sent to these systems for their use and for use by players, officials, other personnel, or other persons. Location aspects of the player location system 20 can include, but are not limited to computer locator 20a, stadium locator 20b, watch locator 20c, band locator 20d, wrist locator 20e, helmet locator 20f, visor locator 20g, RFID locator 20h, emitter locator 20i, in ground locator 20j with buried perimeter wires 20k, 20l, and 20m.

In operation, the player location system 20 includes such functions as determining current and historical player field-location data. The player location system 20 can include use of RF emitter with spaced wire-pair. The wire pair can be buried in ground. The player location system 20 can determine player location through RFID based systems. The player location system can include technology such as based on Zebra Technologies RFID system player emitters and field sensors. The player location system 20 can include player location determination thru player image recognition, player uniform recognition, or player-specific spectrum based recognition. The player location system 20 can transmit/receive information/data to/from field access system, etc.

Turning now to FIG. 2, FIG. 2 depicts some aspects also depicted in FIGS. 1A-1F and discussed above and also below regarding communication between impact sensor system 12, player testing system 14 (having communication system 150), player status system 16, field access system 18, and player location system 20.

Turning now to FIG. 3, player testing system 14 depicted as including communication system 150, which is depicted to include processor 150a, memory 150b, operating system 150c, and device interface 150e.

Processor 150a may include one or more microprocessors, central processing units (“cpu”), a graphics processing units (“gpu”), physics processing units, digital signal processors, network processors, floating point processors, and the other processors. In implementation(s), processor 150a may be a server. In implementation(s), processor 150a may be a distributed-core processor. Although processor 150a can be understood in one sense as depicted as a single processor that is part of a single communication system 150, processor 150a may be multiple processors distributed over one or many communication systems 150, which may or may not be configured to operate together. Processor 150a is illustrated as being configured to execute computer readable instructions in order to execute one or more operations described above.

Further shown in FIG. 3, communication system 150 includes memory 150b, which may include memory, cache memory such as random access memory (RAM), flash memory, synchronous random access memory (SRAM), dynamic random access memory (DRAM), or other types of memory such as read only memory (“ROM”), programmable read only memory (“PROM”), flash memory, hard drives, erasable programmable read-only memory (EPROM), disk-based media, disc-based media, magnetic storage, optical storage, volatile memory, nonvolatile memory, mass storage devices, and any combination thereof. In implementation(s), memory 150b may be at single network site(s) or separated from the communication system 150, e.g., available on different system(s) on a network, wired or wirelessly. For example, in a networked system, there may be many communication systems 150 having memory 150b as located at central server(s) that may be a few feet away or located across an ocean. In implementation(s) memory 150b may be located at multiple network sites, including sites that are distant from each other.

Referring again to FIG. 3, communication system 150 includes operating system 150c, some versions thereof being mobile or otherwise, and may include processing module m10, which may further include modules (some of which are described below), and may further include virtual machines 150d (such as process virtual machines, virtual machines of hardware, virtual machines of virtual machines, Java virtual machines, Dalvik virtual machines, virtual machines for use with Android operating systems such as Samsung or Google mobile devices or for use with other mobile operating systems such as Apple iOS on Microsoft Windows based mobile operating systems, etc.).

As shown also in FIG. 3, communication system 150 can include device interface 150e, which can include user interface 150f, device input 150g, and device output 150h.

In implementation(s), device interface 150e can include any component that allows interaction with its environment. For example, in implementation(s) device interface 150e can include one or more sensors, e.g., a camera, a microphone, an accelerometer, a thermometer, a satellite positioning system (SPS) sensor, a barometer, a humidity sensor, a compass, a gyroscope, a magnetometer, a pressure sensor, an oscillation detector, a light sensor, an inertial measurement unit (IMU), a tactile sensor, a touch sensor, a flexibility sensor, a microelectromechanical system (MEMS), a radio, including a wireless radio, a transmitter, a receiver, an emitter, a broadcaster, etc.

In implementation(s), device interface 150e also may include one or more user interface components, e.g., user interface 150f (e.g., although they are drawn separately, in implementation(s), user interface 150f is a type of device interface 150e)), and in implementation(s) including one or more device inputs 150g and one or more device outputs 150h. User interface 150f may include any hardware, software, firmware, and combination thereof that allows one or more users to interact with communication system 150, and for vice versa. In implementation(s), user interface 150f may include a monitor, screen, touchscreen, liquid crystal display (“LCD”) screen, light emitting diode (“LED”) screen, speaker, handset, earpiece, keyboard, keypad, touchpad, mouse, trackball, remote control, button set, microphone, video camera, still camera, a charge-coupled device (“CCD”) element, a photovoltaic element, etc.

Referring again to FIG. 3, implementation(s) of device interface 150e may include one or more components in addition to or integrated with user interface 150f to provide ways that communication system 150 can input and output information with its environment(s) and/or user(s). These components of device interface 150e for user interface 150f, device input 150g, and/or device output 150h may include one or more sensors, e.g., a camera, a microphone, an accelerometer, a thermometer, a satellite positioning system (SPS) sensor, a barometer, a humidity sensor, a compass, a gyroscope, a magnetometer, a pressure sensor, an oscillation detector, a light sensor, an inertial measurement unit (IMU), a tactile sensor, a touch sensor, a flexibility sensor, a microelectromechanical system (MEMS), a radio, including a wireless radio, a transmitter, a receiver, an emitter, a broadcaster, etc., and other components as well to serve user interface, input and/or output function(s) for device interface 150e such as for user interface 150f, device input 150g and device output 150h.

Further examples of user interface 150f, device input 150g, and/or device output 150h may include any hardware, software, firmware, and combination thereof, to provide capability for a user thereof to interact with communication system 150. Implementation(s) of user interface 150f, device input 150g, and/or device output 150h can include monitor(s), screen(s), touchscreen(s), liquid crystal display (“LCD”) screen(s), light emitting diode (“LED”) screen(s), speaker(s), handset(s), earpiece(s), keyboard(s), keypad(s), touchpad(s), mouse(s), trackball(s), remote control(s), button set(s), microphone(s), video camera(s), still camera(s), a charge-coupled device (“CCD”) element(s), a photovoltaic element(s), etc.

As other examples, implementation(s) of device interface 150e can include including portions for outputting information, inputting information, and/or controlling aspects thereof. Various arrangements such as display window(s), audio emitter(s), tactile interface(s), button(s), slider(s), gesture interface(s), articulation(s), knob(s), icon(s), desktop(s), ribbon(s), bar(s), tool(s), stylus area(s), keypad(s), keyboard(s), and other audio, video, graphic, tactile, etc. input, output, or control aspects can be used. For instance, graphical user interface presentations can be presented upon display surfaces while other input and/or output aspects can be utilized.

Implementations of modules can involve different combinations (limited to patentable subject matter under 35 U.S.C. 101) of one or more aspects from one or more electrical circuitry arrangements and/or one or more aspects from one or more instructions.

In one or more implementations, as shown in FIG. 4, the processing module m10 may include player acceleration data associated with a head module m11.

In one or more implementations, as shown in FIG. 4, the processing module m10 may include game time player status data module m12.

In one or more implementations, as shown in FIG. 4, the processing module m10 may include player brain injury status data module m13.

In one or more implementations, as shown in FIG. 5, module m11 may include acceleration data associated with the head module m102.

In one or more implementations, as shown in FIG. 5, module m102 may include helmet mounted acceleration measurement circuitry module m103.

In one or more implementations, as shown in FIG. 5, module m102 may include mouthguard mounted acceleration measurement circuitry module m104.

In one or more implementations, as shown in FIG. 9, module m12 may include game time player status data module m105.

In one or more implementations, as shown in FIG. 9, module m105 may include location of the sports player with respect to a playing field module m106.

In one or more implementations, as shown in FIG. 9, module m105 may include game clock status data module m107.

In one or more implementations, as shown in FIG. 9, module m105 may include duration of time until the sports player is to resume play module m108.

In one or more implementations, as shown in FIG. 14, module m13 may include wearable brain injury diagnostic circuitry module m109.

In one or more implementations, as shown in FIG. 14, module m109 may include prompting one or more sports player responses module m110.

In one or more implementations, as shown in FIG. 15, module m13 may include human-language-based requests module m111.

In one or more implementations, as shown in FIG. 15, module m111 may include finger-activated input module m112.

In one or more implementations, as shown in FIG. 15, module m111 may include verbal audio input module m113.

In one or more implementations, as shown in FIG. 16, module m13 may include sports player symptomology module m114.

In one or more implementations, as shown in FIG. 16, module m114 may include sports player ocular data module m115.

In one or more implementations, as shown in FIG. 17, module m13 may include field access to the sports player module m116.

In one or more implementations, as shown in FIG. 17, module m116 may include audio-based information regarding sports field access module m117

In one or more implementations, as shown in FIG. 17, module m116 may include visual-based information regarding sports field access module m118.

In one or more implementations, as shown in FIG. 18, module m13 may include summary information based on brain injury status module m119.

In one or more implementations, as shown in FIG. 18, module m119 may include summary information via internet protocols module m120.

In one or more implementations, as shown in FIG. 6, module m11 may include acceleration measurement data module m121.

In one or more implementations, as shown in FIG. 6, module m121 may include acceleration measurement from data thresholds module m122.

In one or more implementations, as shown in FIG. 6, module m121 may include linear acceleration data associated with the head module m123.

In one or more implementations, as shown in FIG. 6, module m121 may include rotational acceleration data associated with the head module m124.

In one or more implementations, as shown in FIG. 6, module m121 may include positional measurement data module m125.

In one or more implementations, as shown in FIG. 7, module m11 may include helmet mounted acceleration measurement circuitry module m126.

In one or more implementations, as shown in FIG. 7, module m126 may include acceleration measurement circuitry mounted module m127.

In one or more implementations, as shown in FIG. 7, module m126 may include acceleration measurement circuitry mounted in a head band module m128.

In one or more implementations, as shown in FIG. 7, module m126 may include measurement of a Riddell Insite football helmet system module m129.

In one or more implementations, as shown in FIG. 7, module m126 may include acceleration measurement circuitry mounted in a skullcap module m130.

In one or more implementations, as shown in FIG. 7, module m126 may include measurement circuitry of a Rebok Checklight MC10 system module m131.

In one or more implementations, as shown in FIG. 8, module m11 may include non-helmet mounted acceleration measurement circuitry module m132.

In one or more implementations, as shown in FIG. 8, module m132 may include measurement circuitry configured for soccer, volleyball, or basketball module m133.

In one or more implementations, as shown in FIG. 8, module m132 may include acceleration measurement data behind ear patch module m134.

In one or more implementations, as shown in FIG. 8, module m132 may include acceleration measurement data RFID system module m135.

In one or more implementations, as shown in FIG. 8, module m132 may include acceleration measurement data wearable emitter module m136.

In one or more implementations, as shown in FIG. 10, module m12 may include team position data module m137.

In one or more implementations, as shown in FIG. 10, module m138 may include engagement status of the team position data module m138.

In one or more implementations, as shown in FIG. 10, module m139 may include presently playing a team position on field of play module m139.

In one or more implementations, as shown in FIG. 11, module m12 may include location data with respect to field of play module m140.

In one or more implementations, as shown in FIG. 11, module m140 may include current field location data module m141.

In one or more implementations, as shown in FIG. 11, module m140 may include historical field location data module m142.

In one or more implementations, as shown in FIG. 11, module m140 may include location data from RF emitter and spaced wire-pair module m143.

In one or more implementations, as shown in FIG. 11, module m140 may include location data from at least in part an RFID system module m144.

In one or more implementations, as shown in FIG. 11, module m140 may include location data from at least in part a GPS system module m145.

In one or more implementations, as shown in FIG. 11, module m140 may include location data from sports player image recognition module m146.

In one or more implementations, as shown in FIG. 12, module m12 may include game time player status data of wearable input circuitry module m147.

In one or more implementations, as shown in FIG. 12, module m147 may include estimate of time remaining before play on field resumes module m148.

In one or more implementations, as shown in FIG. 12, module m147 may include time remaining before the sports player goes back module m149.

In one or more implementations, as shown in FIG. 12, module m147 may include wristband mounted electronic circuitry module m150.

In one or more implementations, as shown in FIG. 12, module m147 may include eyewear mounted electronic circuitry module m151.

In one or more implementations, as shown in FIG. 12, module m147 may include headgear mounted electronic circuitry module m152.

In one or more implementations, as shown in FIG. 12, module m147 may include game statistics of the sports player module m153.

In one or more implementations, as shown in FIG. 13, module m147 may include estimate as to when the sports player will pause module m154.

In one or more implementations, as shown in FIG. 19, module m13 may include brain injury status data via wearable circuitry module m155.

In one or more implementations, as shown in FIG. 19, module m155 may include cue response data via appendage band module m156.

In one or more implementations, as shown in FIG. 19, module m155 may include headgear mounted circuitry module m157.

In one or more implementations, as shown in FIG. 19, module m155 may include smart watch circuitry module m158.

In one or more implementations, as shown in FIG. 19, module m155 may include eyewear mounted circuitry module m159.

In one or more implementations, as shown in FIG. 19, module m155 may include earwear mounted circuitry module m160.

In one or more implementations, as shown in FIG. 20, module m13 may include brain injury status data via mobile device circuitry module m161.

In one or more implementations, as shown in FIG. 20, module m161 may include brain injury status data via laptop circuitry module m162.

In one or more implementations, as shown in FIG. 20, module m161 may include brain injury status data via smart phone circuitry module m163.

In one or more implementations, as shown in FIG. 20, module m161 may include brain injury status data via tablet circuitry module m164.

In one or more implementations, as shown in FIG. 20, module m161 may include time available for obtaining brain injury status data module m165.

In one or more implementations, as shown in FIG. 21, module m13 may include obtaining likelihood of a traumatic brain injury occurrence module m166.

In one or more implementations, as shown in FIG. 21, module m166 may include estimating a likelihood player experienced traumatic brain injury module m167.

In one or more implementations, as shown in FIG. 21, module m166 may include estimating a likelihood experienced traumatic brain injury module m168.

In one or more implementations, as shown in FIG. 21, module m166 may include laser circuitry module m169.

In one or more implementations, as shown in FIG. 21, module m166 may include optical scanning circuitry module m170.

In one or more implementations, as shown in FIG. 22, module m13 may include neurocognitive evaluation data module m171.

In one or more implementations, as shown in FIG. 22, module m171 may include ocular data including: eye tracking data module m172.

In one or more implementations, as shown in FIG. 22, module m171 may include audio recognition data of verbal responses module m173.

In one or more implementations, as shown in FIG. 22, module m171 may include image recognition data of gestured responses module m174.

In one or more implementations, as shown in FIG. 22, module m171 may include data regarding: Sway Balance mobile software data module m175.

In one or more implementations, as shown in FIG. 22, module m171 may include data regarding: King-Devick data module m176.

In one or more implementations, as shown in FIG. 23, module m13 may include multifactor cognitive functioning assessment data module m177.

In one or more implementations, as shown in FIG. 23, module m177 may include balance and orientation symptom evaluation data module m178.

In one or more implementations, as shown in FIG. 23, module m177 may include obtaining: verbal memory data module m179.

In one or more implementations, as shown in FIG. 23, module m177 may include obtaining: response variability data module m180.

In one or more implementations, as shown in FIG. 23, module m177 may include obtaining nonverbal problem solving data module m181.

In one or more implementations, as shown in FIG. 23, module m177 may include obtaining updated baseline markers module m182.

In one or more implementations, as shown in FIG. 23, module m177 may include obtaining progressively detailed testing data module m183.

In one or more implementations, as shown in FIG. 24, module m177 may include obtaining prior response data of the sports player module m184.

In one or more implementations, as shown in FIG. 25, module m13 may include urgency for availability of sports module m185.

In one or more implementations, as shown in FIG. 25, module m185 may include responses to diagnostic cues triggered acceleration thresholds module m186.

In one or more implementations, as shown in FIG. 25, module m185 may include on field responses to diagnostic cues module m187.

In one or more implementations, as shown in FIG. 25, module m185 may include responses during timeout to diagnostic cues module m188.

In one or more implementations, as shown in FIG. 25, module m185 may include responses to visual-based diagnostic cues module m189.

In one or more implementations, as shown in FIG. 25, module m185 may include responses to audio-based diagnostic cues module m190.

In one or more implementations, as shown in FIG. 25, module m185 may include responses to vibratory-based diagnostic cues module m191.

In one or more implementations, as shown in FIG. 26, module m185 may include verbal responses to diagnostic cues module m192.

In one or more implementations, as shown in FIG. 26, module m185 may include gesture-based responses to diagnostic cues module m193.

In one or more implementations, as shown in FIG. 26, module m185 may include eye-movement-based responses to diagnostic cues module m194.

In one or more implementations, as shown in FIG. 26, module m185 may include finger-movement based responses to diagnostic cues module m195.

In one or more implementations, as shown in FIG. 27, module m13 may include brain injury status data based for reporting module m196.

In one or more implementations, as shown in FIG. 27, module m196 may include portion of a team status report module m197.

In one or more implementations, as shown in FIG. 27, module m196 may include in conjunction with historical data module m198.

In one or more implementations, as shown in FIG. 27, module m196 may include outputting: sports player, coach of sports player module m199.

In one or more implementations, as shown in FIG. 27, module m196 may include electronically outputting via smart phone communication module m200.

In one or more implementations, as shown in FIG. 27, module m196 may include electronically outputting via scoreboard circuitry module m201.

An operational flow o10 as shown in FIG. 28 represents example operations related to electronically collecting player acceleration data associated with a head of a sports player; electronically monitoring game time player status data of the sports player; and electronically obtaining player brain injury status data associated with brain injury status of the sports player based at least in part upon the player acceleration data and the game time player status data of the sports player.

FIG. 28 and those figures that follow may have various examples of operational flows, and explanation may be provided with respect to the above-described examples and/or with respect to other examples and contexts. Nonetheless, it should be understood that the operational flows may be executed in a number of other environments and contexts, and/or in modified versions. Furthermore, although the various operational flows are presented in the sequence(s) illustrated, it should be understood that the various operations may be performed in other orders than those which are illustrated, or may be performed concurrently.

In FIG. 28 and those figures that follow, various operations may be depicted in a box-within-a-box manner. Such depictions may indicate that an operation in an internal box may comprise an optional exemplary implementation of the operational step illustrated in one or more external boxes. However, it should be understood that internal box operations may be viewed as independent operations separate from any associated external boxes and may be performed in any sequence with respect to all other illustrated operations, or may be performed concurrently.

Following are a series of flowcharts depicting implementations. For ease of understanding, the flowcharts are organized such that the initial flowcharts present implementations via an example implementation and thereafter the following flowcharts present alternate implementations and/or expansions of the initial flowchart(s) as either sub-component operations or additional component operations building on one or more earlier-presented flowcharts. Those having skill in the art will appreciate that the style of presentation utilized herein (e.g., beginning with a presentation of a flowchart(s) presenting an example implementation and thereafter providing additions to and/or further details in subsequent flowcharts) generally allows for a rapid and easy understanding of the various process implementations. In addition, those skilled in the art will further appreciate that the style of presentation used herein also lends itself well to modular and/or object-oriented program design paradigms.

In one or more implementations, as shown in FIG. 28, the operational flow o10 proceeds to operation o11 for electronically collecting player acceleration data associated with a head of a sports player. Origination of an electronic-semiconductor-transistor-utilizing component group can be accomplished through skilled in the art design choice selection including use of one or more electronic-semiconductor-transistor-containing components and/or subsystems explicitly and/or implicitly referred to herein for the operation o11. One or more non-transitory signal bearing physical media can bear the one or more instructions to direct performance of the operation o11. Furthermore, player acceleration data associated with a head module m11 depicted in FIG. 4 as being included in the processing module m10, performs electronic-semiconductor-transistor-based voltage level switching to carry out the operation o11. Illustratively, in one or more implementations, the operation o11 can be fulfilled, for example, by electronically collecting player acceleration data associated with a head of a sports player (e.g. using acceleration measurement circuitry worn by the sports player such found in the player's helmet, etc.).

In one or more implementations, as shown in FIG. 28, the operational flow o10 proceeds to operation o12 for electronically monitoring game time player status data of the sports player. Origination of an electronic-semiconductor-transistor-utilizing component group can be accomplished through skilled in the art design choice selection including use of one or more electronic-semiconductor-transistor-containing components and/or subsystems explicitly and/or implicitly referred to herein for the operation o12. One or more non-transitory signal bearing physical media can bear the one or more instructions to direct performance of the operation o12. Furthermore, game time player status data module m12 depicted in FIG. 4 as being included in the processing module m10, performs electronic-semiconductor-transistor-based voltage level switching to carry out the operation o12. Illustratively, in one or more implementations, the operation o12 can be fulfilled, for example, by electronically monitoring game time player status data of the sports player (e.g. monitoring player location on and off playing field, track status of playing activity of player to assess what functions player is serving in game, etc.).

In one or more implementations, as shown in FIG. 28, the operational flow o10 proceeds to operation o13 for electronically obtaining player brain injury status data associated with brain injury status of the sports player based at least in part upon the player acceleration data and the game time player status data of the sports player. Origination of an electronic-semiconductor-transistor-utilizing component group can be accomplished through skilled in the art design choice selection including use of one or more electronic-semiconductor-transistor-containing components and/or subsystems explicitly and/or implicitly referred to herein for the operation o13. One or more non-transitory signal bearing physical media can bear the one or more instructions to direct performance of the operation o13. Furthermore, player brain injury status data module m13 depicted in FIG. 4 as being included in the processing module m10, performs electronic-semiconductor-transistor-based voltage level switching to carry out the operation o13. Illustratively, in one or more implementations, the operation o13 can be fulfilled, for example, by electronically obtaining player brain injury status data associated with brain injury status of the sports player based at least in part upon the player acceleration data and the game time player status data of the sports player (e.g. provide response cues to player to solicit responses from the player associated with brain functioning with respect to possible brain injury such as a traumatic brain injury; response cues can include verbal and nonverbal cues using audio, images, vibration, etc. and responses from player can include audible, gesture, finger movement, etc.).

In one or more implementations, as shown in FIG. 29, the operation o11 can include operation o102 for electronically at least partially wirelessly receiving the player acceleration data associated with the head of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o102. One or more non-transitory signal bearing physical media can bear one or more instructions to direct performance of the operation o102. Furthermore, acceleration data associated with the head module m102 depicted in FIG. 5 as being included in the module m11, performs electronic-semiconductor-transistor-based voltage level switching to carry out the operation o102. Illustratively, in one or more implementations, the operation o102 can be fulfilled, for example, by electronically at least partially wirelessly receiving the player acceleration data associated with the head of the sports player (e.g. Over a course of time a head of a player, such as a football player, has received head impacts, acceleration data acquired via helmet-mounted acceleration circuitry, and transmitted to be received through cellular communication device, etc.).

In one or more implementations, as shown in FIG. 29, the operation o102 can include operation o103 for electronically receiving the player acceleration data via helmet mounted acceleration measurement circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o103. One or more non-transitory signal bearing physical media can bear one or more instructions to direct performance of the operation o103. Furthermore, helmet mounted acceleration measurement circuitry module m103 depicted in FIG. 5 as being included in the module m102, performs electronic-semiconductor-transistor-based voltage level switching to carry out the operation o103. Illustratively, in one or more implementations, the operation o103 can be fulfilled, for example, by electronically receiving the player acceleration data via helmet mounted acceleration measurement circuitry (e.g. during a football game, a player receives a series of low intensity head impacts that are each measured by acceleration measurement circuitry integrated with the player's helmet to be wirelessly transmitted for wireless reception, etc.).

In one or more implementations, as shown in FIG. 29, the operation o102 can include operation o104 for electronically receiving the player acceleration data via mouthguard mounted acceleration measurement circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o104. One or more non-transitory signal bearing physical media can bear one or more instructions to direct performance of the operation o104. Furthermore, mouthguard mounted acceleration measurement circuitry module m104 depicted in FIG. 5 as being included in the module m102, performs electronic-semiconductor-transistor-based voltage level switching to carry out the operation o104. Illustratively, in one or more implementations, the operation o104 can be fulfilled, for example, by electronically receiving the player acceleration data via mouthguard mounted acceleration measurement circuitry (e.g. Over a course of time a player, such as a soccer player or wrestling competitor, has received head impacts, acceleration data acquired via mouthguard-mounted acceleration circuitry, and transmitted to be received through cellular communication device, etc.).

In one or more implementations, as shown in FIG. 30, the operation o12 can include operation o105 for electronically at least partially wirelessly receiving the game time player status data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o105. One or more non-transitory signal bearing physical media can bear one or more instructions to direct performance of the operation o105. Furthermore, game time player status data module m105 depicted in FIG. 9 as being included in the module m12, performs electronic-semiconductor-transistor-based voltage level switching to carry out the operation o105. Illustratively, in one or more implementations, the operation o105 can be fulfilled, for example, by electronically at least partially wirelessly receiving the game time player status data of the sports player (e.g. during a soccer game, a player verbally indicates when the player is going to be available for short response cue testing during the game and is transmitted to be received through wireless communication, etc.).

In one or more implementations, as shown in FIG. 30, the operation o105 can include operation o106 for electronically receiving location data regarding location of the sports player with respect to a playing field. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o106. One or more non-transitory signal bearing physical media can bear one or more instructions to direct performance of the operation o106. Furthermore, location of the sports player with respect to a playing field module m106 depicted in FIG. 9 as being included in the module m105, performs electronic-semiconductor-transistor-based voltage level switching to carry out the operation o106. Illustratively, in one or more implementations, the operation o106 can be fulfilled, for example, by electronically receiving location data regarding location of the sports player with respect to a playing field (e.g. During sports event, such as a soccer game or hockey game, location data, such as physical player position on soccer field or hockey stadium, is acquired and transmitted to be received through cellular communication, etc.).

In one or more implementations, as shown in FIG. 30, the operation o105 can include operation o107 for electronically receiving game clock status data. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o107. One or more non-transitory signal bearing physical media can bear one or more instructions to direct performance of the operation o107. Furthermore, game clock status data module m107 depicted in FIG. 9 as being included in the module m105, performs electronic-semiconductor-transistor-based voltage level switching to carry out the operation o107. Illustratively, in one or more implementations, the operation o107 can be fulfilled, for example, by electronically receiving game clock status data (e.g. during a football game the status of the game clock is used to determine when a player can receive response cues to assess brain injury status of the player, etc.).

In one or more implementations, as shown in FIG. 30, the operation o105 can include operation o108 for electronically receiving estimation data regarding at least in part duration of time until the sports player is to resume play. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o108. One or more non-transitory signal bearing physical media can bear one or more instructions to direct performance of the operation o108. Furthermore, duration of time until the sports player is to resume play module m108 depicted in FIG. 9 as being included in the module m105, performs electronic-semiconductor-transistor-based voltage level switching to carry out the operation o108. Illustratively, in one or more implementations, the operation o108 can be fulfilled, for example, by electronically receiving estimation data regarding at least in part duration of time until the sports player is to resume play (e.g. During a sporting event, such as football game, a player is on field waiting for an extended timeout to end, given the nature of the timeout, estimation data of when play is to resume is transmitted to be received through wireless communication, etc.).

In one or more implementations, as shown in FIG. 31, the operation o13 can include operation o109 for electronically obtaining brain injury diagnostic data via wearable brain injury diagnostic circuitry, the electronically obtaining brain injury diagnostic data electronically initiated at least in part by the electronically collecting the player acceleration data and the electronically monitoring the game time player status data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o109. One or more non-transitory signal bearing physical media can bear one or more instructions to direct performance of the operation o109. Furthermore, wearable brain injury diagnostic circuitry module m109 depicted in FIG. 14 as being included in the module m13, performs electronic-semiconductor-transistor-based voltage level switching to carry out the operation o109. Illustratively, in one or more implementations, the operation o109 can be fulfilled, for example, by electronically obtaining brain injury diagnostic data via wearable brain injury diagnostic circuitry, the electronically obtaining brain injury diagnostic data electronically initiated at least in part by the electronically collecting the player acceleration data and the electronically monitoring the game time player status data of the sports player (e.g. during a hockey or football game, audio and visual outputs in a hockey or football helmet of a player gives the player response cues to solicit verbal and eye tracking responses from the player to assess to a degree the player's brain injury status as to whether the player has a brain injury and if so, how severe the injury is; the response cues are generated based at least in part upon history of head impacts of the player as indicated by head acceleration data and availability of the player during the game such as during timeouts, penalties, change of shifts, or other times the player is not actually playing the game during the game, etc.).

In one or more implementations, as shown in FIG. 32, the operation o109 can include operation o110 for electronically displaying at least in part cues for prompting one or more sports player responses. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o110. One or more non-transitory signal bearing physical media can bear one or more instructions to direct performance of the operation o110. Furthermore, prompting one or more sports player responses module m110 depicted in FIG. 14 as being included in the module m109, performs electronic-semiconductor-transistor-based voltage level switching to carry out the operation o110. Illustratively, in one or more implementations, the operation o110 can be fulfilled, for example, by electronically displaying at least in part cues for prompting one or more sports player responses (e.g. Over course of time a player, such as a football player has had a series of very low level head impacts, but has current availability to interact with a diagnostic system involving helmet mounted visual display receives display cue, via visual shield, prompting player response such as answering questions to measure cognitive functioning, etc.).

In one or more implementations, as shown in FIG. 31, the operation o13 can include operation o111 for electronically outputting at least in part human-language-based requests to solicit one or more sports player responses. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o111. One or more non-transitory signal bearing physical media can bear one or more instructions to direct performance of the operation o111. Furthermore, human-language-based requests module m111 depicted in FIG. 15 as being included in the module m13, performs electronic-semiconductor-transistor-based voltage level switching to carry out the operation o111. Illustratively, in one or more implementations, the operation o111 can be fulfilled, for example, by electronically outputting at least in part human-language-based requests to solicit one or more sports player responses (e.g. during a baseball game, response cues are given to a player during a time when the player is not actively involved with the game play of the field through visually displayed English language statements, etc.).

In one or more implementations, as shown in FIG. 33, the operation o111 can include operation o112 for electronically receiving at least in part finger-activated input. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o112. One or more non-transitory signal bearing physical media can bear one or more instructions to direct performance of the operation o112. Furthermore, finger-activated input module m112 depicted in FIG. 15 as being included in the module m111, performs electronic-semiconductor-transistor-based voltage level switching to carry out the operation o112. Illustratively, in one or more implementations, the operation o112 can be fulfilled, for example, by electronically receiving at least in part finger-activated input (e.g. player is prompted to respond using a hand controlled input device that measures response times based on finger movements, etc.).

In one or more implementations, as shown in FIG. 33, the operation o111 can include operation o113 for electronically receiving at least in part verbal audio input. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o113. One or more non-transitory signal bearing physical media can bear one or more instructions to direct performance of the operation o113. Furthermore, verbal audio input module m113 depicted in FIG. 15 as being included in the module m111, performs electronic-semiconductor-transistor-based voltage level switching to carry out the operation o113. Illustratively, in one or more implementations, the operation o113 can be fulfilled, for example, by electronically receiving at least in part verbal audio input (e.g. during a football game, a player receives response cues as English language audible statements via earbuds, etc.).

In one or more implementations, as shown in FIG. 31, the operation o13 can include operation o114 for electronically observing at least in part sports player symptomology. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o114. One or more non-transitory signal bearing physical media can bear one or more instructions to direct performance of the operation o114. Furthermore, sports player symptomology module m114 depicted in FIG. 16 as being included in the module m13, performs electronic-semiconductor-transistor-based voltage level switching to carry out the operation o114. Illustratively, in one or more implementations, the operation o114 can be fulfilled, for example, by electronically observing at least in part sports player symptomology (e.g. football player observed with audio microphones and image scanners to determine if player has any slurred speech or abnormalities with eye movements, etc.).

In one or more implementations, as shown in FIG. 34, the operation o114 can include operation o115 for electronically receiving at least in part sports player ocular data. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o115. One or more non-transitory signal bearing physical media can bear one or more instructions to direct performance of the operation o115. Furthermore, sports player ocular data module m115 depicted in FIG. 16 as being included in the module m114, performs electronic-semiconductor-transistor-based voltage level switching to carry out the operation o115. Illustratively, in one or more implementations, the operation o115 can be fulfilled, for example, by electronically receiving at least in part sports player ocular data (e.g. wirelessly receiving eye movement tracking data from a scanner mounted inside of a football helmet of a player during a football game, etc.).

In one or more implementations, as shown in FIG. 35, the operation o13 can include operation o116 for electronically controlling in least in part sports field access to the sports player based at least in part on the electronically obtaining the player brain injury status data associated with brain injury status of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o116. One or more non-transitory signal bearing physical media can bear one or more instructions to direct performance of the operation o116. Furthermore, field access to the sports player module m116 depicted in FIG. 17 as being included in the module m13, performs electronic-semiconductor-transistor-based voltage level switching to carry out the operation o116. Illustratively, in one or more implementations, the operation o116 can be fulfilled, for example, by electronically controlling in least in part sports field access to the sports player based at least in part on the electronically obtaining the player brain injury status data associated with brain injury status of the sports player (e.g. through use of controlled physical barriers or directional signaling player is excluded from access to field if brain injury status data indicates that player should refrain from play, etc.).

In one or more implementations, as shown in FIG. 36, the operation o116 can include operation o117 for electronically outputting at least in part audio-based information regarding sports field access to the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o117. One or more non-transitory signal bearing physical media can bear one or more instructions to direct performance of the operation o117. Furthermore, audio-based information regarding sports field access module m117 depicted in FIG. 17 as being included in the module m116, performs electronic-semiconductor-transistor-based voltage level switching to carry out the operation o117. Illustratively, in one or more implementations, the operation o117 can be fulfilled, for example, by electronically outputting at least in part audio-based information regarding sports field access to the sports player (e.g. during a baseball game a player responds to response cues that indicate the player has a marginal brain injury status that needs further evaluation, when the player heads to go out on the field the player receives audible instruction through directional field speakers to instead proceed to the trainer to obtain further attention, etc.).

In one or more implementations, as shown in FIG. 36, the operation o116 can include operation o118 for electronically outputting in least in part visual-based information regarding sports field access to the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o118. One or more non-transitory signal bearing physical media can bear one or more instructions to direct performance of the operation o118. Furthermore, visual-based information regarding sports field access module m118 depicted in FIG. 17 as being included in the module m116, performs electronic-semiconductor-transistor-based voltage level switching to carry out the operation o118. Illustratively, in one or more implementations, the operation o118 can be fulfilled, for example, by electronically outputting in least in part visual-based information regarding sports field access to the sports player (e.g. player, such as a football player, may receive information via visual shield, hologram, or projection, regarding return to play, etc.).

In one or more implementations, as shown in FIG. 35, the operation o13 can include operation o119 for electronically reporting summary information based at least in part on the electronically obtaining the player brain injury status data associated with brain injury status of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o119. One or more non-transitory signal bearing physical media can bear one or more instructions to direct performance of the operation o119. Furthermore, summary information based on brain injury status module m119 depicted in FIG. 18 as being included in the module m13, performs electronic-semiconductor-transistor-based voltage level switching to carry out the operation o119. Illustratively, in one or more implementations, the operation o119 can be fulfilled, for example, by electronically reporting summary information based at least in part on the electronically obtaining the player brain injury status data associated with brain injury status of the sports player (e.g. after a football player has been assessed through response cues during a game the brain injury status of the player is reported to friends and staff on the field through wireless communication, etc.).

In one or more implementations, as shown in FIG. 37, the operation o119 can include operation o120 for electronically transmitting the summary information via at least in part one or more internet protocols. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o120. One or more non-transitory signal bearing physical media can bear one or more instructions to direct performance of the operation o120. Furthermore, summary information via internet protocols module m120 depicted in FIG. 18 as being included in the module m119, performs electronic-semiconductor-transistor-based voltage level switching to carry out the operation o120. Illustratively, in one or more implementations, the operation o120 can be fulfilled, for example, by electronically transmitting the summary information via at least in part one or more internet protocols (e.g. At any time before, during, and after a sporting event, such as football game, a summation of player, such as football player, game activity and brain injury status data along with associated acceleration data is transmitted via the internet to parents, coaches, hospital staff, etc.).

In one or more implementations, as shown in FIG. 38, the operation o11 can include operation o121 for electronically receiving the acceleration measurement data from at least in part acceleration measurement circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o121. One or more non-transitory signal bearing physical media can bear one or more instructions to direct performance of the operation o121. Furthermore, acceleration measurement data module m121 depicted in FIG. 6 as being included in the module m11, performs electronic-semiconductor-transistor-based voltage level switching to carry out the operation o121. Illustratively, in one or more implementations, the operation o121 can be fulfilled, for example, by electronically receiving the acceleration measurement data from at least in part acceleration measurement circuitry (e.g. during a baseball game, acceleration circuitry found in a mouthguard of a player measures a head impacts received by the player, etc.).

In one or more implementations, as shown in FIG. 39, the operation o121 can include operation o122 for electronically receiving the acceleration measurement data based at least in part on one or more acceleration measurement data thresholds. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o122. One or more non-transitory signal bearing physical media can bear one or more instructions to direct performance of the operation o122. Furthermore, acceleration measurement from data thresholds module m122 depicted in FIG. 6 as being included in the module m121, performs electronic-semiconductor-transistor-based voltage level switching to carry out the operation o122. Illustratively, in one or more implementations, the operation o122 can be fulfilled, for example, by electronically receiving the acceleration measurement data based at least in part on one or more acceleration measurement data thresholds (e.g. player, such as a football player has received head impacts, but some are only minor low level impacts so consequent acceleration data measured by acceleration circuitry is not significant enough to trigger any diagnostic testing for brain injury status of player, etc.).

In one or more implementations, as shown in FIG. 39, the operation o121 can include operation o123 for electronically receiving the acceleration measurement data as at least in part linear acceleration data associated with the head of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o123. One or more non-transitory signal bearing physical media can bear one or more instructions to direct performance of the operation o123. Furthermore, linear acceleration data associated with the head module m123 depicted in FIG. 6 as being included in the module m121, performs electronic-semiconductor-transistor-based voltage level switching to carry out the operation o123. Illustratively, in one or more implementations, the operation o123 can be fulfilled, for example, by electronically receiving the acceleration measurement data as at least in part linear acceleration data associated with the head of the sports player (e.g. during a hockey game acceleration circuitry mounted in the hockey helmet of a player measures linear acceleration of the hockey player's head during head impacts, etc.).

In one or more implementations, as shown in FIG. 39, the operation o121 can include operation o124 for electronically receiving the acceleration measurement data as at least in part rotational acceleration data associated with the head of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o124. One or more non-transitory signal bearing physical media can bear one or more instructions to direct performance of the operation o124. Furthermore, rotational acceleration data associated with the head module m124 depicted in FIG. 6 as being included in the module m121, performs electronic-semiconductor-transistor-based voltage level switching to carry out the operation o124. Illustratively, in one or more implementations, the operation o124 can be fulfilled, for example, by electronically receiving the acceleration measurement data as at least in part rotational acceleration data associated with the head of the sports player (e.g. a player, such as a football player has received rotational head impacts, such as face-mask tackle, measured via helmet-mounted acceleration circuitry, transmits to be received data through cellular communication device, etc.).

In one or more implementations, as shown in FIG. 40, the operation o121 can include operation o125 for electronically receiving the acceleration measurement data based at least in part on positional measurement data. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o125. One or more non-transitory signal bearing physical media can bear one or more instructions to direct performance of the operation o125. Furthermore, positional measurement data module m125 depicted in FIG. 6 as being included in the module m121, performs electronic-semiconductor-transistor-based voltage level switching to carry out the operation o125. Illustratively, in one or more implementations, the operation o125 can be fulfilled, for example, by electronically receiving the acceleration measurement data based at least in part on positional measurement data (e.g. during a football game position data is taken of a player to determine head impacts experienced by the player, etc.).

In one or more implementations, as shown in FIG. 38, the operation o11 can include operation o126 for electronically receiving the acceleration measurement data from at least in part helmet mounted acceleration measurement circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o126. One or more non-transitory signal bearing physical media can bear one or more instructions to direct performance of the operation o126. Furthermore, helmet mounted acceleration measurement circuitry module m126 depicted in FIG. 7 as being included in the module m11, performs electronic-semiconductor-transistor-based voltage level switching to carry out the operation o126. Illustratively, in one or more implementations, the operation o126 can be fulfilled, for example, by electronically receiving the acceleration measurement data from at least in part helmet mounted acceleration measurement circuitry (e.g. player, such as a hockey player, has received head impacts of various intensities, measured via hockey helmet-mounted acceleration circuitry, transmitted to be received through cellular communication device, etc.).

In one or more implementations, as shown in FIG. 41, the operation o126 can include operation o127 for electronically receiving the acceleration measurement data from at least in part acceleration measurement circuitry mounted at least on one of the following: football helmet, baseball helmet, hockey helmet, lacrosse helmet, ski helmet, skateboard helmet, and bicycle helmet. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o127. One or more non-transitory signal bearing physical media can bear one or more instructions to direct performance of the operation o127. Furthermore, acceleration measurement circuitry mounted module m127 depicted in FIG. 7 as being included in the module m126, performs electronic-semiconductor-transistor-based voltage level switching to carry out the operation o127. Illustratively, in one or more implementations, the operation o127 can be fulfilled, for example, by electronically receiving the acceleration measurement data from at least in part acceleration measurement circuitry mounted at least on one of the following: football helmet, baseball helmet, hockey helmet, lacrosse helmet, ski helmet, skateboard helmet, and bicycle helmet (e.g. during hockey game acceleration circuitry in helmet of player measures accelerations due to head impacts and transmits both low impact and high impact acceleration data for wireless reception, etc.).

In one or more implementations, as shown in FIG. 41, the operation o126 can include operation o128 for electronically receiving the acceleration measurement data from at least in part acceleration measurement circuitry mounted in a head band. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o128. One or more non-transitory signal bearing physical media can bear one or more instructions to direct performance of the operation o128. Furthermore, acceleration measurement circuitry mounted in a head band module m128 depicted in FIG. 7 as being included in the module m126, performs electronic-semiconductor-transistor-based voltage level switching to carry out the operation o128. Illustratively, in one or more implementations, the operation o128 can be fulfilled, for example, by electronically receiving the acceleration measurement data from at least in part acceleration measurement circuitry mounted in a head band (e.g. a player, such as a tennis player, has received head impacts of various intensities a measured via head-band mounted acceleration circuitry, and transmitted to be received wirelessly, etc.).

In one or more implementations, as shown in FIG. 41, the operation o126 can include operation o129 for electronically receiving the acceleration measurement data from at least in part acceleration measurement circuitry of a Riddell Insite football helmet system. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o129. One or more non-transitory signal bearing physical media can bear one or more instructions to direct performance of the operation o129. Furthermore, measurement of a Riddell Insite football helmet system module m129 depicted in FIG. 7 as being included in the module m126, performs electronic-semiconductor-transistor-based voltage level switching to carry out the operation o129. Illustratively, in one or more implementations, the operation o129 can be fulfilled, for example, by electronically receiving the acceleration measurement data from at least in part acceleration measurement circuitry of a Riddell Insite football helmet system (e.g. during football game Riddell Insite acceleration circuitry in helmet of player measures accelerations due to head impacts and transmits both low impact and high impact acceleration data for wireless reception, etc.).

In one or more implementations, as shown in FIG. 42, the operation o126 can include operation o130 for electronically receiving the acceleration measurement data from at least in part acceleration measurement circuitry mounted in a skullcap. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o130. One or more non-transitory signal bearing physical media can bear one or more instructions to direct performance of the operation o130. Furthermore, acceleration measurement circuitry mounted in a skullcap module m130 depicted in FIG. 7 as being included in the module m126, performs electronic-semiconductor-transistor-based voltage level switching to carry out the operation o130. Illustratively, in one or more implementations, the operation o130 can be fulfilled, for example, by electronically receiving the acceleration measurement data from at least in part acceleration measurement circuitry mounted in a skullcap (e.g. a player, such as a soccer player has received head impacts of various intensities measured via skullcap mounted acceleration circuitry and data transmitted to be received through cellular communication device, etc.).

In one or more implementations, as shown in FIG. 42, the operation o126 can include operation o131 for electronically receiving the acceleration measurement data from at least in part acceleration measurement circuitry of a Rebok Checklight MC10 system. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o131. One or more non-transitory signal bearing physical media can bear one or more instructions to direct performance of the operation o131. Furthermore, measurement circuitry of a Rebok Checklight MC10 system module m131 depicted in FIG. 7 as being included in the module m126, performs electronic-semiconductor-transistor-based voltage level switching to carry out the operation o131. Illustratively, in one or more implementations, the operation o131 can be fulfilled, for example, by electronically receiving the acceleration measurement data from at least in part acceleration measurement circuitry of a Rebok Checklight MC10 system (e.g. during soccer game Rebok Checklight MC10 acceleration circuitry worn on a player's head measures accelerations due to head impacts and transmits both low impact and high impact acceleration data for wireless reception, etc.).

In one or more implementations, as shown in FIG. 38, the operation o11 can include operation o132 for electronically receiving the acceleration measurement data from at least in part non-helmet mounted acceleration measurement circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o132. One or more non-transitory signal bearing physical media can bear one or more instructions to direct performance of the operation o132. Furthermore, non-helmet mounted acceleration measurement circuitry module m132 depicted in FIG. 8 as being included in the module m11, performs electronic-semiconductor-transistor-based voltage level switching to carry out the operation o132. Illustratively, in one or more implementations, the operation o132 can be fulfilled, for example, by electronically receiving the acceleration measurement data from at least in part non-helmet mounted acceleration measurement circuitry (e.g. a player, such as a basketball player has received head impacts of various intensities, measured via acceleration circuitry mounted on player in place other than helmet such as an adhesive patch, transmits data to be received through wireless communication, etc.).

In one or more implementations, as shown in FIG. 43, the operation o132 can include operation o133 for electronically receiving the acceleration measurement data from at least in part acceleration measurement circuitry configured for soccer, volleyball, or basketball play. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o133. One or more non-transitory signal bearing physical media can bear one or more instructions to direct performance of the operation o133. Furthermore, measurement circuitry configured for soccer, volleyball, or basketball module m133 depicted in FIG. 8 as being included in the module m132, performs electronic-semiconductor-transistor-based voltage level switching to carry out the operation o133. Illustratively, in one or more implementations, the operation o133 can be fulfilled, for example, by electronically receiving the acceleration measurement data from at least in part acceleration measurement circuitry configured for soccer, volleyball, or basketball play (e.g. during soccer game acceleration circuitry worn on player's head measures accelerations due to head impacts and transmits both low impact and high impact acceleration data for wireless reception, etc.).

In one or more implementations, as shown in FIG. 43, the operation o132 can include operation o134 for electronically receiving the acceleration measurement data from at least in part acceleration measurement circuitry of a x2 biosystems behind ear patch system. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o134. One or more non-transitory signal bearing physical media can bear one or more instructions to direct performance of the operation o134. Furthermore, acceleration measurement data behind ear patch module m134 depicted in FIG. 8 as being included in the module m132, performs electronic-semiconductor-transistor-based voltage level switching to carry out the operation o134. Illustratively, in one or more implementations, the operation o134 can be fulfilled, for example, by electronically receiving the acceleration measurement data from at least in part acceleration measurement circuitry of a x2 biosystems behind ear patch system (e.g. a player, such as a tennis player, uses to measure and transmits acceleration data via x2 biosystems behind ear patch, etc.).

In one or more implementations, as shown in FIG. 43, the operation o132 can include operation o135 for electronically receiving the acceleration measurement data from at least in part acceleration measurement circuitry of a Zebra Technologies RFID system. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o135. One or more non-transitory signal bearing physical media can bear one or more instructions to direct performance of the operation o135. Furthermore, acceleration measurement data RFID system module m135 depicted in FIG. 8 as being included in the module m132, performs electronic-semiconductor-transistor-based voltage level switching to carry out the operation o135. Illustratively, in one or more implementations, the operation o135 can be fulfilled, for example, by electronically receiving the acceleration measurement data from at least in part acceleration measurement circuitry of a Zebra Technologies RFID system (e.g. during football game Zebra Technologies RFID positional based acceleration circuitry measures accelerations due to head impacts and transmits both low impact and high impact acceleration data for wireless reception, etc.).

In one or more implementations, as shown in FIG. 44, the operation o132 can include operation o136 for electronically receiving the acceleration measurement data from at least in part acceleration measurement circuitry including one or more wearable emitter circuitry and field positioned receiver circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o136. One or more non-transitory signal bearing physical media can bear one or more instructions to direct performance of the operation o136. Furthermore, acceleration measurement data wearable emitter module m136 depicted in FIG. 8 as being included in the module m132, performs electronic-semiconductor-transistor-based voltage level switching to carry out the operation o136. Illustratively, in one or more implementations, the operation o136 can be fulfilled, for example, by electronically receiving the acceleration measurement data from at least in part acceleration measurement circuitry including one or more wearable emitter circuitry and field positioned receiver circuitry (e.g. football player movement is measured through use of emitter circuitry worn by the player to transmit location signals to be received by receivers located through the sports field, etc.).

In one or more implementations, as shown in FIG. 45, the operation o12 can include operation o137 for electronically receiving team position data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o137. One or more non-transitory signal bearing physical media can bear one or more instructions to direct performance of the operation o137. Furthermore, team position data module m137 depicted in FIG. 10 as being included in the module m12, performs electronic-semiconductor-transistor-based voltage level switching to carry out the operation o137. Illustratively, in one or more implementations, the operation o137 can be fulfilled, for example, by electronically receiving team position data of the sports player (e.g. during football game positional measurement circuitry worn by a player measures position of player and transmits player positional data for wireless reception, etc.).

In one or more implementations, as shown in FIG. 46, the operation o138 can include operation o138 for electronically receiving engagement status of the sports player at least in part of the team position data. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o138. One or more non-transitory signal bearing physical media can bear one or more instructions to direct performance of the operation o138. Furthermore, engagement status of the team position data module m138 depicted in FIG. 10 as being included in the module m138, performs electronic-semiconductor-transistor-based voltage level switching to carry out the operation o138. Illustratively, in one or more implementations, the operation o138 can be fulfilled, for example, by electronically receiving engagement status of the sports player at least in part of the team position data (e.g. during game, speech data, image data, location data, and other data is collected on a football player, to continually assess and update status of the football player as to whether the player is playing, resting, waiting, hurdling, about to be taken out of the game, etc.

In one or more implementations, as shown in FIG. 46, the operation o139 can include operation o139 for electronically receiving status as to whether the sports player is presently playing a team position on field of play. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o139. One or more non-transitory signal bearing physical media can bear one or more instructions to direct performance of the operation o139. Furthermore, presently playing a team position on field of play module m139 depicted in FIG. 10 as being included in the module m139, performs electronic-semiconductor-transistor-based voltage level switching to carry out the operation o139. Illustratively, in one or more implementations, the operation o139 can be fulfilled, for example, by electronically receiving status as to whether the sports player is presently playing a team position on field of play (e.g. during hockey game, location measurement circuitry in form of cameras in stadium and positional circuitry worn by player determine whether player is on ice, penalty box, or bench and transmits assessment for wireless reception, etc.).

In one or more implementations, as shown in FIG. 45, the operation o12 can include operation o140 for electronically receiving sports player location data with respect to field of play. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o140. One or more non-transitory signal bearing physical media can bear one or more instructions to direct performance of the operation o140. Furthermore, location data with respect to field of play module m140 depicted in FIG. 11 as being included in the module m12, performs electronic-semiconductor-transistor-based voltage level switching to carry out the operation o140. Illustratively, in one or more implementations, the operation o140 can be fulfilled, for example, by electronically receiving sports player location data with respect to field of play (e.g. Through duration of sports event, such as a soccer game or hockey game, location data, such as physical player position on soccer field or hockey stadium, transmits through wireless communication, etc.).

In one or more implementations, as shown in FIG. 47, the operation o140 can include operation o141 for electronically receiving current field location data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o141. One or more non-transitory signal bearing physical media can bear one or more instructions to direct performance of the operation o141. Furthermore, current field location data module m141 depicted in FIG. 11 as being included in the module m140, performs electronic-semiconductor-transistor-based voltage level switching to carry out the operation o141. Illustratively, in one or more implementations, the operation o141 can be fulfilled, for example, by electronically receiving current field location data of the sports player (e.g. during football game, location measurement circuitry in form of cameras in stadium and positional circuitry worn by player determine whether player is on football field and transmits assessment for wireless reception, etc.).

In one or more implementations, as shown in FIG. 47, the operation o140 can include operation o142 for electronically receiving historical field location data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o142. One or more non-transitory signal bearing physical media can bear one or more instructions to direct performance of the operation o142. Furthermore, historical field location data module m142 depicted in FIG. 11 as being included in the module m140, performs electronic-semiconductor-transistor-based voltage level switching to carry out the operation o142. Illustratively, in one or more implementations, the operation o142 can be fulfilled, for example, by electronically receiving historical field location data of the sports player (e.g. Through duration of sports event, such as a soccer game or hockey game, historical game player location data, such as physical player position on soccer field or hockey stadium, and is electronically transmitted to be received for purposes such as assisting with predictive functions, etc.).

In one or more implementations, as shown in FIG. 47, the operation o140 can include operation o143 for electronically receiving location data from at least in part an RF emitter and spaced wire-pair. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o143. One or more non-transitory signal bearing physical media can bear one or more instructions to direct performance of the operation o143. Furthermore, location data from RF emitter and spaced wire-pair module m143 depicted in FIG. 11 as being included in the module m140, performs electronic-semiconductor-transistor-based voltage level switching to carry out the operation o143. Illustratively, in one or more implementations, the operation o143 can be fulfilled, for example, by electronically receiving location data from at least in part an RF emitter and spaced wire-pair (e.g. during football game, location measurement circuitry involving RF emitter circuitry worn by player and buried wire pair located along field perimeter determine whether player has entered or exited field and transmits assessment for wireless reception, etc.).

In one or more implementations, as shown in FIG. 48, the operation o140 can include operation o144 for electronically receiving location data from at least in part an RFID system. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o144. One or more non-transitory signal bearing physical media can bear one or more instructions to direct performance of the operation o144. Furthermore, location data from at least in part an RFID system module m144 depicted in FIG. 11 as being included in the module m140, performs electronic-semiconductor-transistor-based voltage level switching to carry out the operation o144. Illustratively, in one or more implementations, the operation o144 can be fulfilled, for example, by electronically receiving location data from at least in part an RFID system (e.g. Through duration of sports event, such as a soccer game or hockey game, embedded or worn electromagnetic-field radio frequency information location data, such as physical player position on soccer field or hockey stadium, transmits through cellular communication, etc.).

In one or more implementations, as shown in FIG. 48, the operation o140 can include operation o145 for electronically receiving location data from at least in part a GPS system. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o145. One or more non-transitory signal bearing physical media can bear one or more instructions to direct performance of the operation o145. Furthermore, location data from at least in part a GPS system module m145 depicted in FIG. 11 as being included in the module m140, performs electronic-semiconductor-transistor-based voltage level switching to carry out the operation o145. Illustratively, in one or more implementations, the operation o145 can be fulfilled, for example, by electronically receiving location data from at least in part a GPS system (e.g. during soccer game, GPS location measurement worn by player determine whether player is on field playing or on sideline resting and transmits assessment for wireless reception, etc.).

In one or more implementations, as shown in FIG. 48, the operation o140 can include operation o146 for electronically receiving location data from at least in part sports player image recognition. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o146. One or more non-transitory signal bearing physical media can bear one or more instructions to direct performance of the operation o146. Furthermore, location data from sports player image recognition module m146 depicted in FIG. 11 as being included in the module m140, performs electronic-semiconductor-transistor-based voltage level switching to carry out the operation o146. Illustratively, in one or more implementations, the operation o146 can be fulfilled, for example, by electronically receiving location data from at least in part sports player image recognition (e.g. Through duration of sports event, such as a soccer game or hockey game, image recognition location data, such as physical player position on soccer field or hockey stadium, measured via distributed camera system transmits image data to be received and recognized, etc.).

In one or more implementations, as shown in FIG. 45, the operation o12 can include operation o147 for electronically receiving the game time player status data of the sports player from at least in part wearable input circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o147. One or more non-transitory signal bearing physical media can bear one or more instructions to direct performance of the operation o147. Furthermore, game time player status data of wearable input circuitry module m147 depicted in FIG. 12 as being included in the module m12, performs electronic-semiconductor-transistor-based voltage level switching to carry out the operation o147. Illustratively, in one or more implementations, the operation o147 can be fulfilled, for example, by electronically receiving the game time player status data of the sports player from at least in part wearable input circuitry (e.g. during soccer game player inputs verbal responses into an eyewear mounted microphone regarding estimation when player will leave the field and transmits for wireless reception, etc.).

In one or more implementations, as shown in FIG. 49, the operation o147 can include operation o148 for electronically receiving the game time player status data of the sports player associated with in least in part an estimate of time remaining before play on field resumes. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o148. One or more non-transitory signal bearing physical media can bear one or more instructions to direct performance of the operation o148. Furthermore, estimate of time remaining before play on field resumes module m148 depicted in FIG. 12 as being included in the module m147, performs electronic-semiconductor-transistor-based voltage level switching to carry out the operation o148. Illustratively, in one or more implementations, the operation o148 can be fulfilled, for example, by electronically receiving the game time player status data of the sports player associated with in least in part an estimate of time remaining before play on field resumes (e.g. A player, such as a football player, is waiting for a timeout to be completed so game data such as nature of timeout and historical data as far as how long such timeouts usually last are generated to determine whether there is time to transmit one or more response cues to player to assess brain injury status of player, etc.).

In one or more implementations, as shown in FIG. 49, the operation o147 can include operation o149 for electronically receiving the game time player status data of the sports player associated with at least in part an estimate of time remaining before the sports player goes back on to the field. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o149. One or more non-transitory signal bearing physical media can bear one or more instructions to direct performance of the operation o149. Furthermore, time remaining before the sports player goes back module m149 depicted in FIG. 12 as being included in the module m147, performs electronic-semiconductor-transistor-based voltage level switching to carry out the operation o149. Illustratively, in one or more implementations, the operation o149 can be fulfilled, for example, by electronically receiving the game time player status data of the sports player associated with at least in part an estimate of time remaining before the sports player goes back on to the field (e.g. during football game, game clock data, down marker data, and timeout data is used to determine estimate of when earliest time for a player to return to the field to play defense and is transmitted for wireless reception, etc.).

In one or more implementations, as shown in FIG. 49, the operation o147 can include operation o150 for electronically receiving the game time player status data of the sports player at least in part from wristband mounted electronic circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o150. One or more non-transitory signal bearing physical media can bear one or more instructions to direct performance of the operation o150. Furthermore, wristband mounted electronic circuitry module m150 depicted in FIG. 12 as being included in the module m147, performs electronic-semiconductor-transistor-based voltage level switching to carry out the operation o150. Illustratively, in one or more implementations, the operation o150 can be fulfilled, for example, by electronically receiving the game time player status data of the sports player at least in part from wristband mounted electronic circuitry (e.g. during a sports event, such as a soccer game or hockey game, data regarding player playing status such as whether the player is on field playing or is waiting to go on field or is about to go off field for a break is obtained through at least in part a wristband worn by player, etc.).

In one or more implementations, as shown in FIG. 50, the operation o147 can include operation o151 for electronically receiving the game time player status data of the sports player at least in part from eyewear mounted electronic circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o151. One or more non-transitory signal bearing physical media can bear one or more instructions to direct performance of the operation o151. Furthermore, eyewear mounted electronic circuitry module m151 depicted in FIG. 12 as being included in the module m147, performs electronic-semiconductor-transistor-based voltage level switching to carry out the operation o151. Illustratively, in one or more implementations, the operation o151 can be fulfilled, for example, by electronically receiving the game time player status data of the sports player at least in part from eyewear mounted electronic circuitry (e.g. during soccer game camera mounted in player eyewear determines if player is on field and transmits determination for wireless reception, etc.).

In one or more implementations, as shown in FIG. 50, the operation o147 can include operation o152 for electronically receiving the game time player status data of the sports player at least in part from headgear mounted electronic circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o152. One or more non-transitory signal bearing physical media can bear one or more instructions to direct performance of the operation o152. Furthermore, headgear mounted electronic circuitry module m152 depicted in FIG. 12 as being included in the module m147, performs electronic-semiconductor-transistor-based voltage level switching to carry out the operation o152. Illustratively, in one or more implementations, the operation o152 can be fulfilled, for example, by electronically receiving the game time player status data of the sports player at least in part from headgear mounted electronic circuitry (e.g. during a sports event, such as a soccer game or hockey game, data regarding player playing status such as whether the player is on field playing or is waiting to go on field or is about to go off field for a break is obtained through at least in part a helmet mounted input such as microphone worn by player, etc.).

In one or more implementations, as shown in FIG. 50, the operation o147 can include operation o153 for electronically receiving the game time player status data of the sports player associated with at least in part game statistics of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o153. One or more non-transitory signal bearing physical media can bear one or more instructions to direct performance of the operation o153. Furthermore, game statistics of the sports player module m153 depicted in FIG. 12 as being included in the module m147, performs electronic-semiconductor-transistor-based voltage level switching to carry out the operation o153. Illustratively, in one or more implementations, the operation o153 can be fulfilled, for example, by electronically receiving the game time player status data of the sports player associated with at least in part game statistics of the sports player (e.g. during baseball game fielding and batting statistics of a player are transmitted for wireless reception, etc.).

In one or more implementations, as shown in FIG. 51, the operation o147 can include operation o154 for electronically receiving the game time player status data of the sports player associated with at least in part an estimate as to when the sports player will pause from play on the field. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o154. One or more non-transitory signal bearing physical media can bear one or more instructions to direct performance of the operation o154. Furthermore, estimate as to when the sports player will pause module m154 depicted in FIG. 13 as being included in the module m147, performs electronic-semiconductor-transistor-based voltage level switching to carry out the operation o154. Illustratively, in one or more implementations, the operation o154 can be fulfilled, for example, by electronically receiving the game time player status data of the sports player associated with at least in part an estimate as to when the sports player will pause from play on the field (e.g. Through duration of sports event, such as a soccer game or hockey game, data regarding player readiness status, such as player fatigue or what sub-team the player is on, etc.).

In one or more implementations, as shown in FIG. 52, the operation o13 can include operation o155 for electronically obtaining the player brain injury status data via wearable circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o155. One or more non-transitory signal bearing physical media can bear one or more instructions to direct performance of the operation o155. Furthermore, brain injury status data via wearable circuitry module m155 depicted in FIG. 19 as being included in the module m13, performs electronic-semiconductor-transistor-based voltage level switching to carry out the operation o155. Illustratively, in one or more implementations, the operation o155 can be fulfilled, for example, by electronically obtaining the player brain injury status data via wearable circuitry (e.g. during football game cue responses are sent to player via audio speakers worn by player and player responses are obtained via a microphone mounted in player's helmet, etc.).

In one or more implementations, as shown in FIG. 53, the operation o155 can include operation o156 for electronically receiving player cue response data associated with brain injury status of the sports player via appendage band mounted circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o156. One or more non-transitory signal bearing physical media can bear one or more instructions to direct performance of the operation o156. Furthermore, cue response data via appendage band module m156 depicted in FIG. 19 as being included in the module m155, performs electronic-semiconductor-transistor-based voltage level switching to carry out the operation o156. Illustratively, in one or more implementations, the operation o156 can be fulfilled, for example, by electronically receiving player cue response data associated with brain injury status of the sports player via appendage band mounted circuitry (e.g. during a soccer game or hockey game, response cues are transmitted to player through arm band to help assess brain injury status of player data, etc.).

In one or more implementations, as shown in FIG. 53, the operation o155 can include operation o157 for electronically receiving player cue response data associated with brain injury status of the sports player via headgear mounted circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o157. One or more non-transitory signal bearing physical media can bear one or more instructions to direct performance of the operation o157. Furthermore, headgear mounted circuitry module m157 depicted in FIG. 19 as being included in the module m155, performs electronic-semiconductor-transistor-based voltage level switching to carry out the operation o157. Illustratively, in one or more implementations, the operation o157 can be fulfilled, for example, by electronically receiving player cue response data associated with brain injury status of the sports player via headgear mounted circuitry (e.g. during football game cue responses are sent to player via audio speakers in player's helmet and player responses are obtained via a microphone mounted in player's helmet, etc.).

In one or more implementations, as shown in FIG. 53, the operation o155 can include operation o158 for electronically receiving player cue response data associated with brain injury status of the sports player via smart watch circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o158. One or more non-transitory signal bearing physical media can bear one or more instructions to direct performance of the operation o158. Furthermore, smart watch circuitry module m158 depicted in FIG. 19 as being included in the module m155, performs electronic-semiconductor-transistor-based voltage level switching to carry out the operation o158. Illustratively, in one or more implementations, the operation o158 can be fulfilled, for example, by electronically receiving player cue response data associated with brain injury status of the sports player via smart watch circuitry (e.g. during a soccer game or hockey game, response cues are transmitted to player through smart watch circuitry to help assess brain injury status of player, etc.)).

In one or more implementations, as shown in FIG. 54, the operation o155 can include operation o159 for electronically receiving player cue response data associated with brain injury status of the sports player via eyewear mounted circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o159. One or more non-transitory signal bearing physical media can bear one or more instructions to direct performance of the operation o159. Furthermore, eyewear mounted circuitry module m159 depicted in FIG. 19 as being included in the module m155, performs electronic-semiconductor-transistor-based voltage level switching to carry out the operation o159. Illustratively, in one or more implementations, the operation o159 can be fulfilled, for example, by electronically receiving player cue response data associated with brain injury status of the sports player via eyewear mounted circuitry (e.g. during soccer game cue responses are displayed to player via player eyewear mounted display circuitry, etc.).

In one or more implementations, as shown in FIG. 54, the operation o155 can include operation o160 for electronically receiving player cue response data associated with brain injury status of the sports player via earwear mounted circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o160. One or more non-transitory signal bearing physical media can bear one or more instructions to direct performance of the operation o160. Furthermore, earwear mounted circuitry module m160 depicted in FIG. 19 as being included in the module m155, performs electronic-semiconductor-transistor-based voltage level switching to carry out the operation o160. Illustratively, in one or more implementations, the operation o160 can be fulfilled, for example, by electronically receiving player cue response data associated with brain injury status of the sports player via earwear mounted circuitry (e.g. during a soccer game or hockey game, response cues are transmitted to player through earwear circuitry to help assess brain injury status of player, etc.).

In one or more implementations, as shown in FIG. 52, the operation o13 can include operation o161 for electronically obtaining the player brain injury status data via mobile device circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o161. One or more non-transitory signal bearing physical media can bear one or more instructions to direct performance of the operation o161. Furthermore, brain injury status data via mobile device circuitry module m161 depicted in FIG. 20 as being included in the module m13, performs electronic-semiconductor-transistor-based voltage level switching to carry out the operation o161. Illustratively, in one or more implementations, the operation o161 can be fulfilled, for example, by electronically obtaining the player brain injury status data via mobile device circuitry (e.g. during baseball game a player inputs responses to response cues using a tablet while in dugout, etc.).

In one or more implementations, as shown in FIG. 55, the operation o161 can include operation o162 for electronically obtaining the player brain injury status data via laptop circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o162. One or more non-transitory signal bearing physical media can bear one or more instructions to direct performance of the operation o162. Furthermore, brain injury status data via laptop circuitry module m162 depicted in FIG. 20 as being included in the module m161, performs electronic-semiconductor-transistor-based voltage level switching to carry out the operation o162. Illustratively, in one or more implementations, the operation o162 can be fulfilled, for example, by electronically obtaining the player brain injury status data via laptop circuitry (e.g. during a soccer game or hockey game, response cues are transmitted to player through laptop circuitry to help assess brain injury status of player, etc.).

In one or more implementations, as shown in FIG. 55, the operation o161 can include operation o163 for electronically obtaining the player brain injury status data via smart phone circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o163. One or more non-transitory signal bearing physical media can bear one or more instructions to direct performance of the operation o163. Furthermore, brain injury status data via smart phone circuitry module m163 depicted in FIG. 20 as being included in the module m161, performs electronic-semiconductor-transistor-based voltage level switching to carry out the operation o163. Illustratively, in one or more implementations, the operation o163 can be fulfilled, for example, by electronically obtaining the player brain injury status data via smart phone circuitry (e.g. during baseball game a player inputs responses to response cues using a smart phone while in dugout, etc.).

In one or more implementations, as shown in FIG. 55, the operation o161 can include operation o164 for electronically obtaining the player brain injury status data via tablet circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o164. One or more non-transitory signal bearing physical media can bear one or more instructions to direct performance of the operation o164. Furthermore, brain injury status data via tablet circuitry module m164 depicted in FIG. 20 as being included in the module m161, performs electronic-semiconductor-transistor-based voltage level switching to carry out the operation o164. Illustratively, in one or more implementations, the operation o164 can be fulfilled, for example, by electronically obtaining the player brain injury status data via tablet circuitry (e.g. during a soccer game or hockey game, response cues are transmitted to player through tablet circuitry to help assess brain injury status of player, etc.).

In one or more implementations, as shown in FIG. 56, the operation o161 can include operation o165 for electronically estimating amount of time available for obtaining the player brain injury status data based at least in part on game time player status data. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o165. One or more non-transitory signal bearing physical media can bear one or more instructions to direct performance of the operation o165. Furthermore, time available for obtaining brain injury status data module m165 depicted in FIG. 20 as being included in the module m161, performs electronic-semiconductor-transistor-based voltage level switching to carry out the operation o165. Illustratively, in one or more implementations, the operation o165 can be fulfilled, for example, by electronically estimating amount of time available for obtaining the player brain injury status data based at least in part on game time player status data (e.g. during football game status data regarding player participation time on defense is used to estimate when player will be resting on sidelines next, etc.).

In one or more implementations, as shown in FIG. 52, the operation o13 can include operation o166 for electronically obtaining the player brain injury status data with respect to likelihood of a traumatic brain injury occurrence by the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o166. One or more non-transitory signal bearing physical media can bear one or more instructions to direct performance of the operation o166. Furthermore, obtaining likelihood of a traumatic brain injury occurrence module m166 depicted in FIG. 21 as being included in the module m13, performs electronic-semiconductor-transistor-based voltage level switching to carry out the operation o166. Illustratively, in one or more implementations, the operation o166 can be fulfilled, for example, by electronically obtaining the player brain injury status data with respect to likelihood of a traumatic brain injury occurrence by the sports player (e.g. during soccer game or hockey game, responses from player to visual and auditory response cues are obtained to determine a percentage likelihood that the player has a brain injury, etc.).

In one or more implementations, as shown in FIG. 57, the operation o166 can include operation o167 for electronically estimating a likelihood between low to near zero probability that the sports player experienced a recent traumatic brain injury. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o167. One or more non-transitory signal bearing physical media can bear one or more instructions to direct performance of the operation o167. Furthermore, estimating a likelihood player experienced traumatic brain injury module m167 depicted in FIG. 21 as being included in the module m166, performs electronic-semiconductor-transistor-based voltage level switching to carry out the operation o167. Illustratively, in one or more implementations, the operation o167 can be fulfilled, for example, by electronically estimating a likelihood between low to near zero probability that the sports player experienced a recent traumatic brain injury (e.g. during football game response cues of a player are analyzed to determine brain injury status of player as to whether player has low or near zero probability of having a traumatic brain injury at present, etc.).

In one or more implementations, as shown in FIG. 57, the operation o166 can include operation o168 for electronically estimating a likelihood between very high to near certain that the sports player experienced a recent traumatic brain injury. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o168. One or more non-transitory signal bearing physical media can bear one or more instructions to direct performance of the operation o168. Furthermore, estimating a likelihood experienced traumatic brain injury module m168 depicted in FIG. 21 as being included in the module m166, performs electronic-semiconductor-transistor-based voltage level switching to carry out the operation o168. Illustratively, in one or more implementations, the operation o168 can be fulfilled, for example, by electronically estimating a likelihood between very high to near certain that the sports player experienced a recent traumatic brain injury (e.g. Through duration of sports event, such as a soccer game or hockey game, data regarding player brain injury status in respect to likelihood or certainty that player was involved in brain injury traumatic event, is electronically monitored, etc.).

In one or more implementations, as shown in FIG. 57, the operation o166 can include operation o169 for electronically obtaining the player brain injury status data via laser circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o169. One or more non-transitory signal bearing physical media can bear one or more instructions to direct performance of the operation o169. Furthermore, laser circuitry module m169 depicted in FIG. 21 as being included in the module m166, performs electronic-semiconductor-transistor-based voltage level switching to carry out the operation o169. Illustratively, in one or more implementations, the operation o169 can be fulfilled, for example, by electronically obtaining the player brain injury status data via laser circuitry (e.g. during hockey game low level laser scanning related to player eye movements generate data as part of diagnostics to determine level of likelihood that player has a traumatic brain injury, etc.).

In one or more implementations, as shown in FIG. 58, the operation o166 can include operation o170 for electronically obtaining the player brain injury status data via optical scanning circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o170. One or more non-transitory signal bearing physical media can bear one or more instructions to direct performance of the operation o170. Furthermore, optical scanning circuitry module m170 depicted in FIG. 21 as being included in the module m166, performs electronic-semiconductor-transistor-based voltage level switching to carry out the operation o170. Illustratively, in one or more implementations, the operation o170 can be fulfilled, for example, by electronically obtaining the player brain injury status data via optical scanning circuitry (e.g. Through duration of sports event, such as a soccer game or hockey game, data regarding player brain injury status measured via optical scanning circuitry of the player's eye, transmits through cellular communication, etc.).

In one or more implementations, as shown in FIG. 59, the operation o13 can include operation o171 for electronically obtaining neurocognitive evaluation data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o171. One or more non-transitory signal bearing physical media can bear one or more instructions to direct performance of the operation o171. Furthermore, neurocognitive evaluation data module m171 depicted in FIG. 22 as being included in the module m13, performs electronic-semiconductor-transistor-based voltage level switching to carry out the operation o171. Illustratively, in one or more implementations, the operation o171 can be fulfilled, for example, by electronically obtaining neurocognitive evaluation data of the sports player (e.g. during football game a player receives a number of cues such as verbal questions and non-verbal symbolic cues that the player responds to which is then used to assess cognitive ability of the player compared to a baseline for the player, etc.).

In one or more implementations, as shown in FIG. 60, the operation o171 can include operation o172 for electronically receiving sports player ocular data including one or more of the following: eye tracking data, pupil dilation data, pupil synching data. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o172. One or more non-transitory signal bearing physical media can bear one or more instructions to direct performance of the operation o172. Furthermore, ocular data including: eye tracking data module m172 depicted in FIG. 22 as being included in the module m171, performs electronic-semiconductor-transistor-based voltage level switching to carry out the operation o172. Illustratively, in one or more implementations, the operation o172 can be fulfilled, for example, by electronically receiving sports player ocular data including one or more of the following: eye tracking data, pupil dilation data, pupil synching data (e.g. during a hockey game, data regarding player ocular data, such as pupil dilation, eye tracking data, and pupil synching data, using a scanner built into the hockey helmet transmits through cellular communication, etc.).

In one or more implementations, as shown in FIG. 60, the operation o171 can include operation o173 for electronically receiving audio recognition data of sports player verbal responses. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o173. One or more non-transitory signal bearing physical media can bear one or more instructions to direct performance of the operation o173. Furthermore, audio recognition data of verbal responses module m173 depicted in FIG. 22 as being included in the module m171, performs electronic-semiconductor-transistor-based voltage level switching to carry out the operation o173. Illustratively, in one or more implementations, the operation o173 can be fulfilled, for example, by electronically receiving audio recognition data of sports player verbal responses (e.g. during football game, a player responds verbally to a number of response cues in which his verbal responses are analyzed to recognize patterns that may indicate brain injury, etc.).

In one or more implementations, as shown in FIG. 60, the operation o171 can include operation o174 for electronically receiving image recognition data of sports player gestured responses. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o174. One or more non-transitory signal bearing physical media can bear one or more instructions to direct performance of the operation o174. Furthermore, image recognition data of gestured responses module m174 depicted in FIG. 22 as being included in the module m171, performs electronic-semiconductor-transistor-based voltage level switching to carry out the operation o174. Illustratively, in one or more implementations, the operation o174 can be fulfilled, for example, by electronically receiving image recognition data of sports player gestured responses (e.g. during soccer game or hockey game, image recognition data regarding player gestured responses in response to response cues to measure brain injury status is transmitted to be received wirelessly, etc.).

In one or more implementations, as shown in FIG. 61, the operation o171 can include operation o175 for electronically receiving data regarding one or more of the following: Sway Balance mobile software data, Standardized Concussion Assessment Tool (SCAT2) data. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o175. One or more non-transitory signal bearing physical media can bear one or more instructions to direct performance of the operation o175. Furthermore, data regarding: Sway Balance mobile software data module m175 depicted in FIG. 22 as being included in the module m171, performs electronic-semiconductor-transistor-based voltage level switching to carry out the operation o175. Illustratively, in one or more implementations, the operation o175 can be fulfilled, for example, by electronically receiving data regarding one or more of the following: Sway Balance mobile software data, Standardized Concussion Assessment Tool (SCAT2) data (e.g. during baseball game a player undergoes short testing using wearable circuitry to obtain response data to response cues based on SCAT2 testing, etc.).

In one or more implementations, as shown in FIG. 61, the operation o171 can include operation o176 for electronically receiving data regarding one or more of the following: King-Devick data, Balance Error Scoring System (BESS) data, imPACT system data, Dynamic Visual Acuity data. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o176. One or more non-transitory signal bearing physical media can bear one or more instructions to direct performance of the operation o176. Furthermore, data regarding: King-Devick data module m176 depicted in FIG. 22 as being included in the module m171, performs electronic-semiconductor-transistor-based voltage level switching to carry out the operation o176. Illustratively, in one or more implementations, the operation o176 can be fulfilled, for example, by electronically receiving data regarding one or more of the following: King-Devick data, Balance Error Scoring System (BESS) data, imPACT system data, Dynamic Visual Acuity data (e.g. during football game, player is given response cues through a wearable interface based on Balance Error Scoring System to assess brain injury status of player and data is transmitted to be received through cellular communication, etc.).

In one or more implementations, as shown in FIG. 59, the operation o13 can include operation o177 for electronically obtaining multifactor cognitive functioning assessment data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o177. One or more non-transitory signal bearing physical media can bear one or more instructions to direct performance of the operation o177. Furthermore, multifactor cognitive functioning assessment data module m177 depicted in FIG. 23 as being included in the module m13, performs electronic-semiconductor-transistor-based voltage level switching to carry out the operation o177. Illustratively, in one or more implementations, the operation o177 can be fulfilled, for example, by electronically obtaining multifactor cognitive functioning assessment data of the sports player (e.g. during football game, a player receives response cues through wearable circuitry regarding abstract and concrete reasoning to determine cognitive status and possible brain injury, etc.).

In one or more implementations, as shown in FIG. 62, the operation o177 can include operation o178 for electronically obtaining balance and orientation symptom evaluation data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o178. One or more non-transitory signal bearing physical media can bear one or more instructions to direct performance of the operation o178. Furthermore, balance and orientation symptom evaluation data module m178 depicted in FIG. 23 as being included in the module m177, performs electronic-semiconductor-transistor-based voltage level switching to carry out the operation o178. Illustratively, in one or more implementations, the operation o178 can be fulfilled, for example, by electronically obtaining balance and orientation symptom evaluation data of the sports player (e.g. during field hockey game, player is given audio response cues to assess brain injury status based on balance and orientation assessment with data transmitted to be received wirelessly, etc.).

In one or more implementations, as shown in FIG. 62, the operation o177 can include operation o179 for electronically obtaining one or more of the following data of the sports player: verbal memory data, visual memory data, concentration data, short term memory data, working memory data, selective attention span data, sustained attention span data. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o179. One or more non-transitory signal bearing physical media can bear one or more instructions to direct performance of the operation o179. Furthermore, obtaining: verbal memory data module m179 depicted in FIG. 23 as being included in the module m177, performs electronic-semiconductor-transistor-based voltage level switching to carry out the operation o179. Illustratively, in one or more implementations, the operation o179 can be fulfilled, for example, by electronically obtaining one or more of the following data of the sports player: verbal memory data, visual memory data, concentration data, short term memory data, working memory data, selective attention span data, sustained attention span data (e.g. during a football game, a player receives a number of response cues through a tablet interface to test aspects of player memory and attention to determine brain injury status of player, etc.).

In one or more implementations, as shown in FIG. 62, the operation o177 can include operation o180 for electronically obtaining one or more of the following data of the sports player: response variability data, processing speed data, reaction time data. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o180. One or more non-transitory signal bearing physical media can bear one or more instructions to direct performance of the operation o180. Furthermore, obtaining: response variability data module m180 depicted in FIG. 23 as being included in the module m177, performs electronic-semiconductor-transistor-based voltage level switching to carry out the operation o180. Illustratively, in one or more implementations, the operation o180 can be fulfilled, for example, by electronically obtaining one or more of the following data of the sports player: response variability data, processing speed data, reaction time data (e.g. during football game, player is given visual response cues to assess brain injury status based on response variability, processing speed and reaction time assessments for brain injury status of player with data transmitted to be received electronically, etc.).

In one or more implementations, as shown in FIG. 63, the operation o177 can include operation o181 for electronically obtaining nonverbal problem solving data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o181. One or more non-transitory signal bearing physical media can bear one or more instructions to direct performance of the operation o181. Furthermore, obtaining nonverbal problem solving data module m181 depicted in FIG. 23 as being included in the module m177, performs electronic-semiconductor-transistor-based voltage level switching to carry out the operation o181. Illustratively, in one or more implementations, the operation o181 can be fulfilled, for example, by electronically obtaining nonverbal problem solving data of the sports player (e.g. during hockey game, a player submits responses to graphical nonverbal response cues presented to player through a tablet interface, etc.).

In one or more implementations, as shown in FIG. 63, the operation o177 can include operation o182 for electronically obtaining updated baseline markers of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o182. One or more non-transitory signal bearing physical media can bear one or more instructions to direct performance of the operation o182. Furthermore, obtaining updated baseline markers module m182 depicted in FIG. 23 as being included in the module m177, performs electronic-semiconductor-transistor-based voltage level switching to carry out the operation o182. Illustratively, in one or more implementations, the operation o182 can be fulfilled, for example, by electronically obtaining updated baseline markers of the sports player (e.g. during hockey game, player is given response cues to update baseline response markers for the player regarding future assessments of brain injury status of the player, etc.).

In one or more implementations, as shown in FIG. 63, the operation o177 can include operation o183 for electronically obtaining progressively detailed testing data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o183. One or more non-transitory signal bearing physical media can bear one or more instructions to direct performance of the operation o183. Furthermore, obtaining progressively detailed testing data module m183 depicted in FIG. 23 as being included in the module m177, performs electronic-semiconductor-transistor-based voltage level switching to carry out the operation o183. Illustratively, in one or more implementations, the operation o183 can be fulfilled, for example, by electronically obtaining progressively detailed testing data of the sports player (e.g. during football, a player is presented an initial set of response cues and based on player's response, a latter period during the game the player is presented with a more detailed set of response cues to respond based on the player's initial responses, etc.).

In one or more implementations, as shown in FIG. 64, the operation o177 can include operation o184 for electronically obtaining additional data of the sports player based on prior response data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o184. One or more non-transitory signal bearing physical media can bear one or more instructions to direct performance of the operation o184. Furthermore, obtaining prior response data of the sports player module m184 depicted in FIG. 24 as being included in the module m177, performs electronic-semiconductor-transistor-based voltage level switching to carry out the operation o184. Illustratively, in one or more implementations, the operation o184 can be fulfilled, for example, by electronically obtaining additional data of the sports player based on prior response data of the sports player (e.g. during baseball game, player is given response cues based on prior responses of the player earlier in the game to further assess brain injury status of the player, etc.).

In one or more implementations, as shown in FIG. 59, the operation o13 can include operation o185 for electronically obtaining the player brain injury status data based in part on urgency for the obtaining as indicated by player acceleration data and availability of sports player as indicated by the game time player status data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o185. One or more non-transitory signal bearing physical media can bear one or more instructions to direct performance of the operation o185. Furthermore, urgency for availability of sports module m185 depicted in FIG. 25 as being included in the module m13, performs electronic-semiconductor-transistor-based voltage level switching to carry out the operation o185. Illustratively, in one or more implementations, the operation o185 can be fulfilled, for example, by electronically obtaining the player brain injury status data based in part on urgency for the obtaining as indicated by player acceleration data and availability of sports player as indicated by the game time player status data of the sports player (e.g. during football game, a player receives a number of low to medium level head impacts so that during a time soon thereafter when the player is on the sidelines the player receives a set of response cues to determine if the head impacts affected the player's brain injury status, etc.).

In one or more implementations, as shown in FIG. 65, the operation o185 can include operation o186 for electronically receiving sports player responses to diagnostic cues guided by one or more triggered player acceleration thresholds. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o186. One or more non-transitory signal bearing physical media can bear one or more instructions to direct performance of the operation o186. Furthermore, responses to diagnostic cues triggered acceleration thresholds module m186 depicted in FIG. 25 as being included in the module m185, performs electronic-semiconductor-transistor-based voltage level switching to carry out the operation o186. Illustratively, in one or more implementations, the operation o186 can be fulfilled, for example, by electronically receiving sports player responses to diagnostic cues guided by one or more triggered player acceleration thresholds (e.g. during a football game, a player receives a number of head impacts, none of which are extreme, but are recorded to determine a cumulative exposure that breaches a threshold to trigger response cues to be transmitted to the player through audio or visual interfaces to determine brain injury status of player via the player's responses, etc.).

In one or more implementations, as shown in FIG. 65, the operation o185 can include operation o187 for electronically receiving sports player on field responses to diagnostic cues. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o187. One or more non-transitory signal bearing physical media can bear one or more instructions to direct performance of the operation o187. Furthermore, on field responses to diagnostic cues module m187 depicted in FIG. 25 as being included in the module m185, performs electronic-semiconductor-transistor-based voltage level switching to carry out the operation o187. Illustratively, in one or more implementations, the operation o187 can be fulfilled, for example, by electronically receiving sports player on field responses to diagnostic cues (e.g. during a football game, a field timeout allows sufficient time for the player to response to response cues to determine to a degree the brain injury status of the player, etc.).

In one or more implementations, as shown in FIG. 65, the operation o185 can include operation o188 for electronically receiving sports player responses from on football field during timeout to diagnostic cues. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o188. One or more non-transitory signal bearing physical media can bear one or more instructions to direct performance of the operation o188. Furthermore, responses during timeout to diagnostic cues module m188 depicted in FIG. 25 as being included in the module m185, performs electronic-semiconductor-transistor-based voltage level switching to carry out the operation o188. Illustratively, in one or more implementations, the operation o188 can be fulfilled, for example, by electronically receiving sports player responses from on football field during timeout to diagnostic cues (e.g. during a timeout on a football field, a player is provided response cues through the player's audio-visual helmet system in which the player responds to, etc.).

In one or more implementations, as shown in FIG. 66, the operation o185 can include operation o189 for electronically receiving sports player on field responses to visual-based diagnostic cues. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o189. One or more non-transitory signal bearing physical media can bear one or more instructions to direct performance of the operation o189. Furthermore, responses to visual-based diagnostic cues module m189 depicted in FIG. 25 as being included in the module m185, performs electronic-semiconductor-transistor-based voltage level switching to carry out the operation o189. Illustratively, in one or more implementations, the operation o189 can be fulfilled, for example, by electronically receiving sports player on field responses to visual-based diagnostic cues (e.g. during football game, a sports player responds to visual response cues displayed in the visor of the player's helmet and is transmitted to be received wirelessly, etc.).

In one or more implementations, as shown in FIG. 66, the operation o185 can include operation o190 for electronically receiving sports player on field responses to audio-based diagnostic cues. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o190. One or more non-transitory signal bearing physical media can bear one or more instructions to direct performance of the operation o190. Furthermore, responses to audio-based diagnostic cues module m190 depicted in FIG. 25 as being included in the module m185, performs electronic-semiconductor-transistor-based voltage level switching to carry out the operation o190. Illustratively, in one or more implementations, the operation o190 can be fulfilled, for example, by electronically receiving sports player on field responses to audio-based diagnostic cues (e.g. during a timeout in a soccer game, player on the field is given a series of audio response cues through the player's earbud system that the player responds to through gestures that are recorded by image sensors on the field in order to assess to a degree brain injury status of the player, etc.).

In one or more implementations, as shown in FIG. 66, the operation o185 can include operation o191 for electronically receiving sports player on field responses to vibratory-based diagnostic cues. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o191. One or more non-transitory signal bearing physical media can bear one or more instructions to direct performance of the operation o191. Furthermore, responses to vibratory-based diagnostic cues module m191 depicted in FIG. 25 as being included in the module m185, performs electronic-semiconductor-transistor-based voltage level switching to carry out the operation o191. Illustratively, in one or more implementations, the operation o191 can be fulfilled, for example, by electronically receiving sports player on field responses to vibratory-based diagnostic cues (e.g. during hockey game a player off the ice responds to vibrational response cues that part of a glove worn by the player by flexing certain fingers in response to the vibrations, etc.).

In one or more implementations, as shown in FIG. 67, the operation o185 can include operation o192 for electronically receiving sports player on field verbal responses to diagnostic cues. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o192. One or more non-transitory signal bearing physical media can bear one or more instructions to direct performance of the operation o192. Furthermore, verbal responses to diagnostic cues module m192 depicted in FIG. 26 as being included in the module m185, performs electronic-semiconductor-transistor-based voltage level switching to carry out the operation o192. Illustratively, in one or more implementations, the operation o192 can be fulfilled, for example, by electronically receiving sports player on field verbal responses to diagnostic cues (e.g. during a timeout in a football game, player on the field is given a series of audio response cues through the player's earbud system that the player responds to through verbal input into a microphone mounted in the player's helmet in order to assess to a degree brain injury status of the player, etc.).

In one or more implementations, as shown in FIG. 67, the operation o185 can include operation o193 for electronically receiving sports player on field gesture-based responses to diagnostic cues. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o193. One or more non-transitory signal bearing physical media can bear one or more instructions to direct performance of the operation o193. Furthermore, gesture-based responses to diagnostic cues module m193 depicted in FIG. 26 as being included in the module m185, performs electronic-semiconductor-transistor-based voltage level switching to carry out the operation o193. Illustratively, in one or more implementations, the operation o193 can be fulfilled, for example, by electronically receiving sports player on field gesture-based responses to diagnostic cues (e.g. during football game, a player receives verbal response cues through helmet audio and responds by gesturing before field cameras, etc.).

In one or more implementations, as shown in FIG. 67, the operation o185 can include operation o194 for electronically receiving sports player on field eye-movement-based responses to diagnostic cues. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o194. One or more non-transitory signal bearing physical media can bear one or more instructions to direct performance of the operation o194. Furthermore, eye-movement-based responses to diagnostic cues module m194 depicted in FIG. 26 as being included in the module m185, performs electronic-semiconductor-transistor-based voltage level switching to carry out the operation o194. Illustratively, in one or more implementations, the operation o194 can be fulfilled, for example, by electronically receiving sports player on field eye-movement-based responses to diagnostic cues (e.g. during a timeout in a basketball game, player on the court is given a series of visual response cues through the player's eyewear system that the player responds to through eye movement that is recorded by image sensors in the player's eyewear to assess to a degree brain injury status of the player, etc.).

In one or more implementations, as shown in FIG. 68, the operation o185 can include operation o195 for electronically receiving sports player on field finger-movement based responses to diagnostic cues. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o195. One or more non-transitory signal bearing physical media can bear one or more instructions to direct performance of the operation o195. Furthermore, finger-movement based responses to diagnostic cues module m195 depicted in FIG. 26 as being included in the module m185, performs electronic-semiconductor-transistor-based voltage level switching to carry out the operation o195. Illustratively, in one or more implementations, the operation o195 can be fulfilled, for example, by electronically receiving sports player on field finger-movement based responses to diagnostic cues (e.g. during a baseball game, a player uses a computer input device that receives tapping from all fingers of a hand to respond to response cues, etc.).

In one or more implementations, as shown in FIG. 70, the operation o13 can include operation o196 for electronically outputting the player brain injury status data based for reporting. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o196. One or more non-transitory signal bearing physical media can bear one or more instructions to direct performance of the operation o196. Furthermore, brain injury status data based for reporting module m196 depicted in FIG. 27 as being included in the module m13, performs electronic-semiconductor-transistor-based voltage level switching to carry out the operation o196. Illustratively, in one or more implementations, the operation o196 can be fulfilled, for example, by electronically outputting the player brain injury status data based for reporting (e.g. upon receipt of brain injury status data, reporting is transmitted to parents and friends in stadium or at their homes, etc.).

In one or more implementations, as shown in FIG. 69, the operation o196 can include operation o197 for electronically outputting as a portion of a team status report. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o197. One or more non-transitory signal bearing physical media can bear one or more instructions to direct performance of the operation o197. Furthermore, portion of a team status report module m197 depicted in FIG. 27 as being included in the module m196, performs electronic-semiconductor-transistor-based voltage level switching to carry out the operation o197. Illustratively, in one or more implementations, the operation o197 can be fulfilled, for example, by electronically outputting as a portion of a team status report (e.g. during a football game, brain injury status is determined for players of a team as the players have time to respond to response cues and as the brain injury status of the players is updated it is reported to parents of the players via wireless internet communication, etc.).

In one or more implementations, as shown in FIG. 69, the operation o196 can include operation o198 for electronically outputting in conjunction with historical data of the sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o198. One or more non-transitory signal bearing physical media can bear one or more instructions to direct performance of the operation o198. Furthermore, in conjunction with historical data module m198 depicted in FIG. 27 as being included in the module m196, performs electronic-semiconductor-transistor-based voltage level switching to carry out the operation o198. Illustratively, in one or more implementations, the operation o198 can be fulfilled, for example, by electronically outputting in conjunction with historical data of the sports player (e.g. upon receipt of brain injury status data, reporting is transmitted to include brain injury status data of the current season for the player, etc.).

In one or more implementations, as shown in FIG. 69, the operation o196 can include operation o199 for electronically outputting to one or more of the following: sports player, coach of sports player, trainer of sports player, medic of sports player, parent of sports player, referee of sports player, hospital of sports player. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o199. One or more non-transitory signal bearing physical media can bear one or more instructions to direct performance of the operation o199. Furthermore, outputting: sports player, coach of sports player module m199 depicted in FIG. 27 as being included in the module m196, performs electronic-semiconductor-transistor-based voltage level switching to carry out the operation o199. Illustratively, in one or more implementations, the operation o199 can be fulfilled, for example, by electronically outputting to one or more of the following: sports player, coach of sports player, trainer of sports player, medic of sports player, parent of sports player, referee of sports player, hospital of sports player (e.g. during a baseball game, brain injury status is determined for players of a team as the players have time to respond to response cues and as the brain injury status of the players is updated it is reported via wireless internet communication to parents or friends of players, etc.).

In one or more implementations, as shown in FIG. 70, the operation o196 can include operation o200 for electronically outputting via smart phone communication. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o200. One or more non-transitory signal bearing physical media can bear one or more instructions to direct performance of the operation o200. Furthermore, electronically outputting via smart phone communication module m200 depicted in FIG. 27 as being included in the module m196, performs electronic-semiconductor-transistor-based voltage level switching to carry out the operation o200. Illustratively, in one or more implementations, the operation o200 can be fulfilled, for example, by electronically outputting via smart phone communication (e.g. upon receipt of brain injury status data, reporting is transmitted to parents and friends in stadium or at their homes via iPhone or Samsung Galaxy smart phone communication, etc.).

In one or more implementations, as shown in FIG. 70, the operation o196 can include operation o201 for electronically outputting via scoreboard circuitry. Origination of a physically tangible electronic-semiconductor-transistor-utilizing component group can be accomplished through skilled in the art design choice selection including use of one or more electronic-semiconductor-transistor-containing components and/or subsystems explicitly and/or implicitly referred to herein for the operation o201. One or more non-transitory signal bearing physical media can bear one or more instructions to direct performance of the operation o201. Furthermore, electronically outputting via scoreboard circuitry module m201 depicted in FIG. 27 as being included in the module m196, performs electronic-semiconductor-transistor-based voltage level switching to carry out the operation o201. Illustratively, in one or more implementations, the operation o201 can be fulfilled, for example, by electronically outputting via scoreboard circuitry (e.g. during a football game, brain injury status is determined for players of a team as the players have time to respond to response cues and as the brain injury status of the players is updated to be displayed in graphical form on a portion of the field scoreboard, etc.).

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

To the extent that formal outline headings are present in this application, it is to be understood that the outline headings are for presentation purposes, and that different types of subject matter may be discussed throughout the application (e.g., device(s)/structure(s) may be described under process(es)/operations heading(s) and/or process(es)/operations may be discussed under structure(s)/process(es) headings; and/or descriptions of single topics may span two or more topic headings). Hence, any use of formal outline headings in this application is for presentation purposes, and is not intended to be in any way limiting.

Throughout this application, examples and lists are given, with parentheses, the abbreviation “e.g.,” or both. Unless explicitly otherwise stated, these examples and lists are merely exemplary and are non-exhaustive. In most cases, it would be prohibitive to list every example and every combination. Thus, smaller, illustrative lists and examples are used, with focus on imparting understanding of the claim terms rather than limiting the scope of such terms.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Although one or more users maybe shown and/or described herein, e.g., in FIG. 1, and other places, as a single illustrated figure, those skilled in the art will appreciate that one or more users may be representative of one or more human users, robotic users (e.g., computational entity), and/or substantially any combination thereof (e.g., a user may be assisted by one or more robotic agents) unless context dictates otherwise. Those skilled in the art will appreciate that, in general, the same may be said of “sender” and/or other entity-oriented terms as such terms are used herein unless context dictates otherwise.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

This application may make reference to one or more trademarks, e.g., a word, letter, symbol, or device adopted by one manufacturer or merchant and used to identify and/or distinguish his or her product from those of others. Trademark names used herein are set forth in such language that makes clear their identity, that distinguishes them from common descriptive nouns, that have fixed and definite meanings, or, in many if not all cases, are accompanied by other specific identification using terms not covered by trademark. In addition, trademark names used herein have meanings that are well-known and defined in the literature, or do not refer to products or compounds for which knowledge of one or more trade secrets is required in order to divine their meaning. All trademarks referenced in this application are the property of their respective owners, and the appearance of one or more trademarks in this application does not diminish or otherwise adversely affect the validity of the one or more trademarks. All trademarks, registered or unregistered, that appear in this application are assumed to include a proper trademark symbol, e.g., the circle R or bracketed capitalization (e.g., [trademark name]), even when such trademark symbol does not explicitly appear next to the trademark. To the extent a trademark is used in a descriptive manner to refer to a product or process, that trademark should be interpreted to represent the corresponding product or process as of the date of the filing of this patent application.

Throughout this application, the terms “in an embodiment,” ‘in one embodiment,” “in some embodiments,” “in several embodiments,” “in at least one embodiment,” “in various embodiments,” and the like, may be used. Each of these terms, and all such similar terms should be construed as “in at least one embodiment, and possibly but not necessarily all embodiments,” unless explicitly stated otherwise. Specifically, unless explicitly stated otherwise, the intent of phrases like these is to provide non-exclusive and non-limiting examples of implementations of the invention. The mere statement that one, some, or may embodiments include one or more things or have one or more features, does not imply that all embodiments include one or more things or have one or more features, but also does not imply that such embodiments must exist. It is a mere indicator of an example and should not be interpreted otherwise, unless explicitly stated as such.

The one or more instructions discussed herein may be, for example, computer executable and/or logic-implemented instructions. In some implementations, signal-bearing medium as articles of manufacture may store the one or more instructions. In some implementations, the signal bearing medium may include a computer-readable medium. In some implementations, the signal-bearing medium may include a recordable medium. In some implementations, the signal-bearing medium may include a communication medium.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

1. A computationally-implemented system, comprising:
circuitry for electronically collecting player acceleration data associated with a head of a sports player;
circuitry for electronically monitoring game time player status data of the sports player; and
circuitry for electronically obtaining player brain injury status data associated with brain injury status of the sports player based at least in part upon the player acceleration data and the game time player status data of the sports player.

2. The computationally-implemented system of claim 1, wherein said circuitry for electronically collecting player acceleration data associated with a head of a sports player comprises:
circuitry for electronically at least partially wirelessly receiving the player acceleration data associated with the head of the sports player.

3. The computationally-implemented system of claim 2, wherein said circuitry for electronically at least partially wirelessly receiving the player acceleration data associated with the head of the sports player comprises:
circuitry for electronically receiving the player acceleration data via helmet mounted acceleration measurement circuitry.

4. The computationally-implemented system of claim 2, wherein said circuitry for electronically at least partially wirelessly receiving the player acceleration data associated with the head of the sports player comprises:
circuitry for electronically receiving the player acceleration data via mouthguard mounted acceleration measurement circuitry.

5. The computationally-implemented system of claim 1, wherein said circuitry for electronically monitoring game time player status data of the sports player comprises:
circuitry for electronically at least partially wirelessly receiving the game time player status data of the sports player.

6. The computationally-implemented system of claim 5, wherein said circuitry for electronically at least partially wirelessly receiving the game time player status data of the sports player comprises:
circuitry for electronically receiving location data regarding location of the sports player with respect to a playing field.

7. The computationally-implemented system of claim 5, wherein said circuitry for electronically at least partially wirelessly receiving the game time player status data of the sports player comprises:
circuitry for electronically receiving game clock status data.

8. The computationally-implemented system of claim 5, wherein said circuitry for electronically at least partially wirelessly receiving the game time player status data of the sports player comprises:
circuitry for electronically receiving estimation data regarding at least in part duration of time until the sports player is to resume play.

9.-10. (canceled)

11. The computationally-implemented system of claim 1, wherein said circuitry for electronically obtaining player brain injury status data associated with brain injury status of the sports player based at least in part upon the player acceleration data and the game time player status data of the sports player comprises:
circuitry for electronically outputting at least in part human-language-based requests to solicit one or more sports player responses.

12.-15. (canceled)

16. The computationally-implemented system of claim 1, wherein said circuitry for electronically obtaining player brain injury status data associated with brain injury status of the sports player based at least in part upon the player acceleration data and the game time player status data of the sports player comprises:
circuitry for electronically controlling in least in part sports field access to the sports player based at least in part on the electronically obtaining the player brain injury status data associated with brain injury status of the sports player.

17. The computationally-implemented system of claim 16, wherein said circuitry for electronically controlling in least in part sports field access to the sports player based at least in part on the electronically obtaining the player brain injury status data associated with brain injury status of the sports player comprises:
circuitry for electronically outputting at least in part audio-based information regarding sports field access to the sports player.

18. The computationally-implemented system of claim 16, wherein said circuitry for electronically controlling in least in part sports field access to the sports player based at least in part on the electronically obtaining the player brain injury status data associated with brain injury status of the sports player comprises:
circuitry for electronically outputting in least in part visual-based information regarding sports field access to the sports player.

19.-31. (canceled)

32. The computationally-implemented system of claim 1, wherein said circuitry for electronically collecting player acceleration data associated with a head of a sports player comprises:
circuitry for electronically receiving the acceleration measurement data from at least in part non-helmet mounted acceleration measurement circuitry.

33. The computationally-implemented system of claim 32, wherein said circuitry for electronically receiving the acceleration measurement data from at least in part non-helmet mounted acceleration measurement circuitry comprises:
circuitry for electronically receiving the acceleration measurement data from at least in part acceleration measurement circuitry configured for soccer, volleyball, or basketball play.

34.-46. (canceled)

47. The computationally-implemented system of claim 1, wherein said circuitry for electronically monitoring game time player status data of the sports player comprises:
circuitry for electronically receiving the game time player status data of the sports player from at least in part wearable input circuitry.

48.-60. (canceled)

61. The computationally-implemented system of claim 1, wherein said circuitry for electronically obtaining player brain injury status data associated with brain injury status of the sports player based at least in part upon the player acceleration data and the game time player status data of the sports player comprises:
circuitry for electronically obtaining the player brain injury status data via mobile device circuitry.

62.-65. (canceled)

66. The computationally-implemented system of claim 1, wherein said circuitry for electronically obtaining player brain injury status data associated with brain injury status of the sports player based at least in part upon the player acceleration data and the game time player status data of the sports player comprises:
circuitry for electronically obtaining the player brain injury status data with respect to likelihood of a traumatic brain injury occurrence by the sports player.

67.-70. (canceled)

71. The computationally-implemented system of claim 1, wherein said circuitry for electronically obtaining player brain injury status data associated with brain injury status of the sports player based at least in part upon the player acceleration data and the game time player status data of the sports player comprises:
circuitry for electronically obtaining neurocognitive evaluation data of the sports player.

72.-101. (canceled)

102. A computationally-implemented method, comprising:
electronically collecting player acceleration data associated with a head of a sports player;
electronically monitoring game time player status data of the sports player; and
electronically obtaining player brain injury status data associated with brain injury status of the sports player based at least in part upon the player acceleration data and the game time player status data of the sports player.

103.-203. (canceled)

204. A device defined by a computational language comprising:
one or more interchained physical machines ordered for electronically collecting player acceleration data associated with a head of a sports player;
one or more interchained physical machines ordered for electronically monitoring game time player status data of the sports player; and
one or more interchained physical machines ordered for electronically obtaining player brain injury status data associated with brain injury status of the sports player based at least in part upon the player acceleration data and the game time player status data of the sports player.

205. (canceled)
